(19) United States
(12) Patent Application Publication
(10) Pub. No.: US 2016/0271268 A1
(43) Pub. Date: Sep. 22, 2016

(54) NUCLEIC ACID NANOSTRUCTURES FOR IN VIVO AGENT DELIVERY

(71) Applicant: DANA-FARBER CANCER INSTITUTE, INC., Boston, MA (US)

(72) Inventors: William M. Shih, Cambridge, MA (US); Nandhini Ponnuswamy, Cambridge, MA (US); Maartje Bastings, Boston, MA (US)

(73) Assignee: Dana-Farber Cancer Institute, Inc., Boston, MA (US)

(21) Appl. No.: 15/034,566

(22) PCT Filed: Nov. 7, 2014
(86) PCT No.: PCT/US2014/064659

§ 371 Date: May 5, 2016

Related U.S. Application Data
(60) Provisional application No. 62/021,257, filed on Jul. 7, 2014.
(60) Provisional application No. 62/021,256, filed on Jul. 7, 2014.
(60) Provisional application No. 61/901,820, filed on Nov. 8, 2013.

(51) Int. Cl. A61K 47/48; A61K 39/39; A61K 47/34; C07H 21/04

(52) U.S. Cl. CPC A61K 47/48315; C07H 21/04; A61K 39/39; A61K 47/34; A61K 47/48369; A61K 2039/6025

(57) ABSTRACT

The present disclosure provides, in some aspects, nucleic acid nanostructures subsaturated with polyamine polymers. The present disclosure also provides, in some aspects, nucleic acid nanocapsules that include a first nucleic acid nanostructure bound to a second nucleic acid nanostructure through a pH-sensitive interface, thereby forming a nanocapsule having an exterior surface and an interior compartment having an interior surface.

### RELATED APPLICATIONS

This application claims the benefit under 35 U.S.C. §119(e) of U.S. provisional application No. 61/901,820, filed Nov. 8, 2013, U.S. provisional application No. 62/021,256, filed Jul. 7, 2014, and U.S. provisional application No. 62/021,257, filed Jul. 7, 2014, the teachings of each of which are incorporated by reference herein.

### FEDERALLY SPONSORED RESEARCH

This invention was made with Government support under Grant No. 1 DP2 OD004641-01 awarded by National Institutes of Health, under Grant No. CCF-1317291 awarded by National Science Foundation, and under W911NF-12-1-0420 awarded by Multidisciplinary University Research Initiative, U.S. Army Research Office. The Government has certain rights in the invention.

### FIELD OF THE INVENTION

The present disclosure relates to the field of nucleic acid nanotechnology. Some embodiments of the present disclosure relate to nucleic acid nanostructures linked to polyamine polymers.

### BACKGROUND OF INVENTION

Nucleic acid nanostructures have great potential in biomedical applications, for example, as drug delivery vehicles. The structures are biodegradable, can be functionalized site-specifically, and can be engineered to undergo allosteric conformational changes, allowing for precise interactions with target molecules and cells. Currently, however, the biomedical application of nucleic acid nanostructures is hindered due to nucleic acid degradation and architectural instability under physiological conditions. For example, use of nucleic acid nanostructures for vaccine delivery, such as cancer vaccine delivery, is particularly problematic. Cancer vaccines promote tumor regression by activating dendritic cells (DCs) to drive the propagation of helper-T lymphocytes and cytotoxic-T lymphocytes that recognize tumor-associated antigens. To overcome the immunosuppressive microenvironment of tumors, an effective vaccine must produce a sustained and potent induction of DCs and T cells. Simple antigens alone, in contrast to many intact pathogens, often do not trigger robust or specific dendritic cell activation, especially for producing the Th1 response required for a vigorous cellular immune response. Thus, some vaccines, or vaccine delivery vehicles, are produced or formulated with properties or other agents that trigger robust or specific DC activation. Due to nucleic acid degradation and architectural instability, use of nucleic acid nanostructures as vaccines, or as vaccine delivery vehicles, has been limited, as they are not able to produce a sustained and potent induction of DCs and T cells.

### SUMMARY OF INVENTION

The present disclosure provides, in some embodiments, nucleic acid nanostructures linked to polyamine polymers that “protect” the nanostructures from, among other things, the adverse effects of low salt environments. In some embodiments, nucleic acid nanostructures also linked to poly(ethylene imine) (PEI) and polyethylene glycol (PEG) copolymers (“PEI-PEG copolymers). Some embodiments of the invention are based, at least in part, on the surprising discovery that the structural integrity of nucleic acid nanostructures can be maintained, even under physiological conditions (e.g., including low salt conditions), by linking the structures to polyamine polymers, or a combination of polyamine polymers and PEI-PEG copolymers. Unexpectedly, when nucleic acid nanostructures are “subsaturated” with polyamine polymers (and in some embodiments, with a combination of polyamine polymers and PEI-PEG copolymers), the architecture of the nanostructures is more stable, and the nucleic acids are more resistant to nuclease degradation, relative to nanostructures without polyamine polymers. The degree of polyamine polymer saturation or, alternatively, the ratio of polyamine polymers to nanostructures impacts nanostructure stability. In some embodiments, the degree of polyamine polymer and PEI-PEG copolymer saturation or, alternatively, the ratio of polyamine polymers and PEI-PEG copolymers to nanostructures impacts nanostructure stability.

Thus, various aspects of the present disclosure provide nucleic acid nanostructures (e.g., nancocapsules with a capsule-like shape) subsaturated with polyamine polymers. Nucleic acid nanostructures are herein considered to be “subsaturated” with polyamine polymers if less than 100% of the phosphates of a nucleic acid nanostructure backbone are linked (e.g., covalently or non-covalently) to amines of polyamine polymers. In some embodiments, less than 95%, less than 90%, less than 80%, less than 70% or less than 60% of the phosphates of nucleic acid nanostructure are linked (e.g., covalently or non-covalently) to amines of the polyamine polymers. In some embodiments, 5% to 95% or 10% to 95% of the phosphates of a nucleic acid nanostructure backbone are linked (e.g., covalently or non-covalently) to amines of polyamine polymers. In some embodiments, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, or 10% to 25% of the phosphates of a nucleic acid nanostructure backbone are linked (e.g., covalently or non-covalently) to amines of polyamine polymers.

In some embodiments, nucleic acid nanostructures further comprise poly(ethylene imine)-polyethylene glycol (PEI-PEG) copolymers. In some embodiments, nucleic acid nanostructures are subsaturated with a combination of polyamine polymers and PEI-PEG copolymers. Nucleic acid nanostructures are herein considered to be “subsaturated” with a combination of polyamine polymers and PEI-PEG copolymers if less than 100% of the phosphates of a nucleic acid nanostructure backbone are linked (e.g., covalently or non-covalently) to amines of polyamine polymers and/or amines of the PEI-PEG copolymers. In some embodiments, less than 95%, less than 90%, less than 80%, less than 70% or less than 60% of the phosphates of nucleic acid nanostructure are linked (e.g., covalently or non-covalently) to amines of the polyamine polymers and/or amines of PEI-PEG copolymers. In some embodiments, 5% to 95% or 10% to 95% of the phosphates of a nucleic acid nanostructure backbone are linked (e.g., covalently or non-covalently) to amines of polyamine polymers and/or amines PEI-PEG copolymers. In some embodiments, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, or 10% to 25% of the phosphates of a nucleic acid nanostructure backbone are linked (e.g., covalently or non-covalently) to amines of polyamine polymers and/or amines PEI-PEG copolymers.

In some embodiments, the ratio of polyamine polymers (e.g., polylysine polymers) to PEI-PEG copolymers is 10:1, 9:1, 8:1, 7:1, 6:1, 5:1, 4:1, 3:1, 2:1 or 1:1. In some embodiments, the ratio of PEI-PEG copolymers to polyamine polymers (e.g., polylysine polymers) is 10:1, 9:1, 8:1, 7:1, 6:1, 5:1, 4:1, 3:1, 2:1 or 1:1.

In some embodiments, nucleic acid nanostructures comprise (e.g., are subsaturated with) a combination of polyamine polymers and copolymers.

Nucleic acid nanostructures may comprise deoxyribonucleic acid (DNA) or ribonucleic acid (RNA). In some embodiments, nucleic acid nanostructures comprise single-stranded plasmid DNA (e.g., single-stranded M13 plasmid DNA).

In some embodiments, nucleic acid nanostructures are two- or three-dimensional. For example, nucleic acid nanostructures may be one of many defined and predetermined shapes such as, for example, a cuboidal shape, a cylindrical shape, an irregular shape or abstract shape.

In some embodiments, nucleic acid nanostructures are rationally designed. A nucleic acid nanostructure is herein considered to be “rationally designed” if the nucleic acids that form the nanostructure are selected based on pre-determined, predictable nucleotide base pairing interactions that direct nucleic acid hybridization (for a review of rational design of DNA nanostructures, see, e.g., Feldkamp U., et al. Angew Chem Int Ed Engl. 2006 Mar. 13; 45(12):1856-76, incorporated herein by reference). In some instances, nucleic acid nanostructures may be referred to as nucleic acid nanoarchitectures (e.g., DNA nanoarchitectures). A nanocapsule, rationally designed to resemble the shape of a capsule, is one example of a particular nucleic acid nanoarchitecture.

It should be appreciated that nucleic acid nanostructures of the present disclosure, in some embodiments, do not include condensed nucleic acid.

It should be appreciated that nucleic acid nanostructures of the present disclosure, in some embodiments, do not include coding nucleic acid. That is, in some embodiments, nucleic acid nanostructures of the present disclosure are “non-coding” nucleic acid nanostructures (i.e., do not include coding nucleic acids). In some embodiments, less than 50% of the nucleic acid sequence in a nucleic acid nanostructure include coding nucleic acid. For example, less than 45%, less than 40%, less than 35%, less than 30%, less than 25%, less than 20%, less than 15%, less than 10% or less than 5% of a nucleic acid nanostructure may include coding nucleic acid sequence.

In some embodiments, nucleic acid nanostructures do not include circular plasmid DNA. In some embodiments, less than 50% of the nucleic acid sequence in a nucleic acid nanostructure include circular plasmid DNA. For example, in terms of nucleic acid length or mass, at least 50% of the nucleic acid sequence used to form or that contributes to the nucleic acid nanostructure is present as a circular plasmid DNA. In some embodiments, less than 45%, less than 40%, less than 35%, less than 30%, less than 25%, less than 20%, less than 15%, less than 10% or less than 5% of a nucleic acid nanostructure includes circular plasmid DNA.

In some embodiments, nucleic acid nanostructures are not encapsulated by or coated with (e.g., linked to) lipids. In some embodiments, less than 50% of the nucleotides in a nucleic acid nanostructure are linked to lipids. For example, less than 45%, less than 40%, less than 35%, less than 30%, less than 25%, less than 20%, less than 15%, less than 10% or less than 5% of nucleotides in a nucleic acid nanostructure may be linked to lipids.

Polyamine polymers, in some embodiments, comprise amino acids. Amino acids may comprise, for example, amine-containing side chains (such amino acids are referred to herein as “amine-containing amino acids.” In some embodiments, polyamine polymers comprise lysine.

In some embodiments, polyamine polymers comprise or consist of peptides. Peptides, in some embodiments, comprise at least 10%, at least 25%, at least 50%, at least 75% or at least 90% lysine or other amine-containing amino acid. Lysines (i.e., lysine amino acids) of polyamine polymers, in some embodiments, are separated from each other by at least one, at least two, at least three, or more, non-lysine amino acids or non-amine-containing amino acids.

In some embodiments, polyamine polymers are polylysine homopolymers (e.g., peptides that consist of lysine). In some embodiments, polyamine polymers are polyarginine homopolymers (e.g., peptides that consist of arginine). In some embodiments, polyamine polymers are polyhistidine homopolymers (e.g., peptides that consist of histidine).

In some embodiments, polyamine polymers are branched.

Polyamine polymers, in some embodiments, comprise or consist of 4 to 100 amino acids, 5 to 75 amino acids, 4 to 50 amino acids, 4 to 25 amino acids, or 4 to 15 amino acids such as amine-containing amino acids. In some embodiments, polyamine polymers comprise or consist of 6, 8, 10 or 12 amino acids such as amine-containing amino acids.

In some embodiments, polyamine polymers comprise spermine.

In some embodiments, nucleic acid nanostructures of the present disclosure comprise one or more groups of Formula (I) or a pharmaceutically acceptable and/or quaternary salt thereof covalently attached thereto:

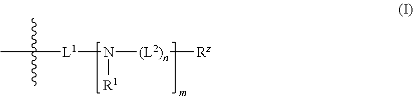

or one or more compounds of Formula (II) or a pharmaceutically acceptable salt and/or quaternary thereof non-covalently associated therewith:

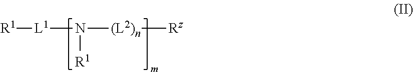

wherein:

L¹ is a direct covalent bond or a linker group comprising any one or combination of optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, and optionally substituted heteroarylene;

each R¹ is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, or a nitrogen protecting group; or

R¹ is a group of formula:

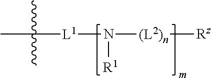

each L² is independently a linker selected from any one or combination of optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, and optionally substituted heteroarylene;

each R^{z} is independently hydrogen, —N(R^{A})₂, —OR^{A}, —SR^{A}, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting if attached to a nitrogen atom, an oxygen protecting group if attached to an oxygen atom, or a sulfur protecting group if attached to a sulfur atom;

each R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting if attached to a nitrogen atom, an oxygen protecting group if attached to an oxygen atom, or a sulfur protecting group if attached to a sulfur atom, or two R^{A} groups attached to a nitrogen atom are joined to form an optionally substituted heterocyclic ring or optionally substituted heteroaryl ring.

n is an integer between 1 and 100,000, inclusive; and

m is an integer between 1 and 100,000, inclusive.

In some embodiments, L² is optionally substituted alkylene, optionally substituted alkenylene, or optionally substituted alkynylene. In some embodiments, L² is an optionally substituted alkylene of formula —(CH2)p-, wherein p is an integer between 1 and 10, inclusive.

In some embodiments, L² is —C(R^{A})—C(═O)—.

In some embodiments, R^{A} of —C(R^{A})—C(═O)— is an optionally substituted alkyl such as a lysine side chain.

Various other aspects of the present disclosure provide nucleic acid compositions that comprise any of the foregoing nucleic acid nanostructures and a solution that comprises less than 10 mM magnesium (Mg²⁺). For example, a solution may comprise 0.1 mM to 0.9 mM Mg²⁺, or 0.6 mM Mg²⁺. In some embodiments, a solution may comprise nanomolar concentrations of Mg²⁺. For example, a solution may comprise 1 nM to 100 nM Mg²⁺. Such low salt concentrations are particularly advantageous, for instance, for use in the field of material science, where high salt can lead to aggregation of metal particles.

In some embodiments, a solution further comprises 0.5 mM to 1.5 mM calcium (Ca²⁺). For example, the solution may comprise 0.9 mM Ca²⁺. In some embodiments, a solution comprises 1.2 mM Ca²⁺.

Other aspects of the present disclosure provide pH-sensitive nucleic acid (e.g., DNA) nanostructures, such as nanocapsules, that function, for example, as carriers for in vivo delivery of agents. A “nucleic acid nanocapsule,” also referred to herein for simplicity as a “nanocapsule,” is a composite three-dimensional nucleic acid nanostructure (e.g., comprising two or more nucleic acid nanostructures) having an exterior surface and an interior compartment for encapsulation of, for example, agents. In some embodiments, nanocapsules are carriers of agents, including adjuvants, for vaccination. Nanocapsules, as provided herein, can be loaded with at least one agent (e.g., antigenic peptide, an RNA interference molecule, adjuvant and/or tracking dye), referred to herein as “cargo,” and targeted to a particular cell type, such as, for example, dendritic cells. Upon entry into the endosome of a cell (e.g., by cell endocytosis of the capsule), a pH-sensitive nanocapsule is triggered by a change in environmental pH to release its cargo. Unlike other nucleic acid nanostructures, a nanocapsule of the present disclosure offers its cargo protection from degradation, in some embodiments, by sequestering the cargo inside a closed compartment of the nanocapsule as the nanocapsule travels to its target cells. Additionally, in some embodiments, the nanocapsule itself is encapsulated by a protective coating (e.g., polyamine polymers, or a combination of polyamine polymers and PEI-PEG copolymers).

Nanocapsules of the present disclosure, in some embodiments, permit a higher concentration of agent delivery to cells in vivo relative to other nucleic acid nanostructure technologies. This higher concentration of delivery, in some embodiments, is the result of a “peg board” configuration of nucleic acid nanocapsules that permits high-density decoration (e.g., one agent per 65 nm²) of the interior of the nanocapsule with agent(s) and encapsulation of agent(s) in the interior compartment of the nanocapsule. A nanocapsule is considered to be “decorated” with agent (e.g., antigen and/or targeting molecules) if the agent is associated with (e.g., covalently or non-covalently linked to) the interior surface or exterior surface of the nanocapsule.

Targeting of nanocapsules is achieved, in some embodiments, by high-density decoration of their exterior surface with targeting molecules (e.g., antibodies or antibody fragments) that bind specifically to cell-type-specific antigens and/or receptors. For example, nanocapsules may be decorated with single chain antibody fragments (scFv) that specifically bind to DEC205, which is enriched on target dendritic cells.

The pH-sensitive opening and release of cargo in a cell (e.g., in an endosomal compartment) depends on two different mechanisms built into some nanocapsules. The first mechanism depends on the presence of partially-complementary pH-responsive single-stranded nucleic acid “handles” and “anti-handles” present at the interface of two nucleic acid nanostructures that, together, form at least part of a nanocapsule. The second mechanism depends on the presence of pH-responsive nucleic acids that function as linkers, linking agent(s) to a surface (e.g., interior and/or exterior) surface of a nanocapsule.

Aspects of the present disclosure provide nucleic acid nanocapsules that comprise a first nucleic acid nanostructure linked to a second nucleic acid nanostructure through a pH-sensitive interface, and an interior compartment formed by linkage of the first nucleic acid nanostructure to the second nucleic acid nanostructure.

In some embodiments, the first nucleic acid nanostructure comprises pH-sensitive single-stranded nucleic acid handles, and the second nucleic acid nanostructure comprises single-stranded nucleic acid anti-handles that are partially complementary to the pH-sensitive handles, and the first nucleic acid nanostructure is linked to the second nucleic acid nanostructure through hybridization of the pH-sensitive handles to the anti-handles. It should be understood that the interaction between handle and anti-handles are pH sensitive due to the pH-sensitive nature of the handles.

In some embodiments, the pH-sensitive handles comprise the sequence of SEQ ID NO: 1.

In some embodiments, the anti-handles comprise the sequence of SEQ ID NO: 2 or SEQ ID NO: 3.

In some embodiments, a nanocapsule has two ends, and each end of the nanocapsule has an opening of less than 10 nm in diameter. In some embodiments, each end of the nanocapsule has an opening of 2 nm to 10 nm (e.g., 2, 3, 4, 5, 6, 7, 8, 9 or 10 nm) in diameter.

In some embodiments, a nucleic acid nanocapsule further comprises an (e.g., at least one) agent linked to an interior surface and/or an exterior surface of the nanocapsule. In some embodiments, a nucleic acid nanocapsule further comprises at least two agents linked to an interior surface and/or an exterior surface of the nanocapsule. In some embodiments, the agent is linked to an interior surface and/or an exterior surface of the nanocapsule through hybridization of complementary single-stranded nucleic acids. In some embodiments, the agent is bound to an interior surface and/or an exterior surface of the nanocapsule through hybridization of partially complementary pH-sensitive handles and anti-handles.

In some embodiments, the agent is a targeting molecule linked to the exterior surface of a nanocapsule. A targeting molecule may be, for example, an antibody, an antibody fragment or a ligand.

In some embodiments, the agent is a therapeutic agent, a prophylactic agent, a diagnostic agent and/or an adjuvant. The antigen may be, for example, a peptide antigen.

In some embodiments, the agent is an adjuvant. The adjuvant may be, for example, a CpG oligonucleotide.

In some embodiments, a nucleic acid nanocapsule further comprises polyamine polymers and/or copolymers of cationic poly(ethylene imine) and polyethylene glycol. In some embodiments, the nanocapsule is subsaturated with the polyamine polymers and/or copolymers of cationic poly(ethylene imine) and polyethylene glycol. In some embodiments, the nanocapsule is subsaturated with a combination of polyamine polymers and copolymers of cationic poly(ethylene imine) and polyethylene glycol.

In some embodiments, the first nucleic acid nanostructure and/or the second nucleic acid nanostructure is in the form of a cylinder.

Aspects of the present disclosure provide compositions that comprise a nucleic acid nanocapsule, as provided herein.

In some embodiments, compositions further comprise a delivery vehicle. In some embodiments, the delivery vehicle is a polymeric gel.

Aspects of the present disclosure provide methods that comprise administering to a subject a nucleic acid nanocapsule or a composition comprising a nanocapsule, as provided herein.

Aspects of the present disclosure provide methods that comprise delivering to cells (e.g., dendritic cells) a nucleic acid nanocapsule, as provided herein. In some embodiments, a nucleic acid nanocapsule is delivered to cells (e.g., dendritic cells) in vivo.

Aspects of the present disclosure provide kits that comprise a first nucleic acid nanostructure comprising pH-sensitive single-stranded nucleic acid handles, and a second nucleic acid nanostructure comprising single-stranded nucleic acid anti-handles that are partially complementary to the pH-sensitive handles, wherein in an aqueous solution having a pH of greater than 6 (e.g., pH greater than 6.5, greater than 7, greater than 7.5), the first nucleic acid nanostructure attaches to the second nucleic acid nanostructure through hybridization of the pH-sensitive handles to the anti-handles, thereby forming a nucleic acid nanocapsule having an internal compartment.

In some embodiments, kits further comprise an aqueous solution having a pH of greater than 6 (e.g., pH greater than 6.5, greater than 7, greater than 7.5).

In some embodiments, the first and second nucleic acid nanostructures comprise pH-sensitive handles.

In some embodiments, pH-sensitive handles comprise the sequence of SEQ ID NO: 1 or a sequence that has at least 80%, 85%, 90%, 95%, 96%, 97%, 98% or 99% identity to SEQ ID NO:1.

In some embodiments, kits further comprise an agent linked to anti-handles that are partially complementary (e.g., at least 80%, 85%, 90%, 95%, 96%, 97%, 98% or 99% complementary) to the pH-sensitive handles.

In some embodiments, kits further comprise pH-sensitive handles and agents linked to anti-handles that are partially complementary (e.g., at least 80%, 85%, 90%, 95%, 96%, 97%, 98% or 99% complementary) to the pH-sensitive handles.

Aspects of the present disclosure provide methods of delivering a vaccine to a subject, comprising delivering (e.g., administering) to the subject a nanocapsule comprising an antigen that activates or stimulates dendritic cells (e.g., to drive the propagation of helper-T lymphocytes and cytotoxic-T lymphocytes). In some embodiments, the nanocapsules further comprise a targeting agent (e.g., antibody or antibody fragment) and/or an adjuvant. In some embodiments, the targeting agent is an antibody or antibody fragment that binds (e.g., binds specifically) to DEC205 or other cell surface marker/receptor of dendritic cells.

### BRIEF DESCRIPTION OF DRAWINGS

FIG. 1 shows a non-limiting example of the effect of spermine molecules on the structural integrity of DNA nanostructures in low-magnesium buffers;

FIG. 2 shows a non-limiting example of the effect of polylysine polymers on the structural integrity of DNA nanostructures in no-magnesium buffers;

FIG. 3 shows a non-limiting example of the effect of polylysine polymers on the structural integrity of DNA nanostructures of various dimensions (samples run on a gel with magnesium);

FIG. 4 shows a non-limiting example of the effect of polylysine polymers on the structural integrity of DNA nanostructures of various dimensions (samples run on a gel without magnesium);

FIG. 5 shows a non-limiting example of a transmission electron microscopy (TEM) analysis of polylysine polymer-induced stability of DNA nanostructures in low-magnesium buffers;

FIG. 6 shows a non-limiting example comparing the effects of polylysine polymers and polyarginine polymers on the structural integrity of DNA nanostructures;

FIG. 7 shows a non-limiting example comparing the effects of different lengths of polylysine polymers on the structural integrity of DNA nanostructures;

FIG. 8 shows a non-limiting example comparing the effects of polylysine polymer length on the respective polylysine polymer concentration required to maintain the structural integrity of DNA nanostructures;

FIG. 9 shows a non-limiting example comparing the effects of polylysine polymer length with respect to thermal stability of DNA nanostructures;

FIG. 10 shows a non-limiting example comparing the effects of polylysine polymer length with respect to nuclease stability of DNA nanostructures;

FIG. 11 shows a non-limiting example comparing the effects of polylysine polymer length with respect to nuclease stability of DNA nanostructures in fresh cell culture media; and

FIGS. 12A-12C show non-limiting examples of polyamine polymers for use in accordance with the present disclosure.

FIGS. 13A and 13B show the effect of length of oligolysine on the negative/positive ratio. FIG. 13A: 100 μL of 1 nM DNA nanostructure in 5 mM Tris, 1 mM EDTA, 6 mM Mg+/−×μM oligolysine (Kₙ; n=6, 8, 10, 12, 20) were dialyzed in 1 L of 5 mM Tris, 1 mM EDTA, pH=7.2 for 18 hours. Longer oligolysines have higher binding affinity to DNA and hence can stabilize DNA nanostructure at high negative/positive ratio. FIG. 13B: TEM images of DNA nanostructure coated with different lengths of oligolysine, stained with 2% uranyl formate.

FIGS. 14A and 14B show oligolysine induced resistance to nuclease degradation. FIG. 14A: 10 μL of freshly prepared cell media in RPMI-1640 was added to DNA nanostructure (1 nM) and incubated at 37° C. for different time intervals. The samples were loaded onto a 2% agarose gel composed of 0.5×TBE and 11 mM Mg. FIG. 14B: Effect of length of oligolysine towards resistance to nuclease degradation in minutes. Coating with oligolysine gives higher resistance to nuclease degradation compared to the naked origami. Moreover, shorter oligolysines can offer higher nuclease resistance as they can afford high (−)/(+) ratio.

FIGS. 15A and 15B show oligolysine induced toxicity to dendritic cells. FIG. 15A: Freshly harvested dendritic cells were incubated with 1.5 μg/mL K₂₀ for different time intervals. The cells appeared healthy when viewed under a microscope. FIG. 15B: Confocal image of dendritic cells incubated with oligolysine (K₂₀) peptide-coated, cy5 labeled DNA nanostructure. Uptake into cells was observed, however, after a few hours, the cells began to burst, and at 24 hours, very few mature cells were observed.

FIGS. 16A and 16B show synthesis and characterization of PEI-PEG complexes. FIG. 16A: Reaction scheme depicting the synthesis of PEI-PEG complexes. Different molar ratios of NHS activated PEG was reacted with PEI to form PEI-PEG complexes with varying amounts of primary amines. FIG. 16B: Distribution of size (mean diameter) with respect to different percentage of PEG coupling. The mean diameter of complexes was measured using Dynamic Light Scalering (DLS).

FIG. 17 shows a schematic representation of multilayered coating of DNA nanostructures. Oligolysine (K₂₀) peptides are depicted by gray squiggly lines, and on interaction with the DNA nanostructure (cylinder), form a uniform coating. The PEI-PEG 50 complexes are shown as a gray circles within a circles, and on interaction with DNA nanostructure, they form a uniform coating.

FIGS. 18A-18D show TEM analysis of multilayered DNA nanostructures. TEM images of (FIG. 18A) naked DNA nanostructure, (FIG. 18B) DNA nanostructure coated with K₂₀, (FIG. 18C) DNA nanostructure coated with only PEI-PEG 50 and (FIG. 18D) DNA nanostructure coated with both K₂₀ and PEI-PEG 50. The samples were stained with 2% uranyl formate. Coating with only K₂₀ preserves the structure, and coating with only PEI-PEG 50 alters the DNA nanostructure. However, coating with both K₂₀ and PEI-PEG 50 maintained structural integrity of the DNA nanostructure.

FIGS. 19A-19D show oligolysine induced resistance to nuclease degradation. 10 μL of freshly prepared cell media in RPMI-1640 was added to DNA nanostructure and incubated at 37° C. for different time intervals. The samples were loaded onto a 2% agarose gel composed of 0.5×TBE and 11 mM Mg. (FIG. 19A) naked origami, (FIG. 19B) origami coated with K₂₀, (FIG. 19C) origami coated with only PEI-PEG 50 and (FIG. 19D) origami coated with both K₂₀ and PEI-PEG 50. Oligolysine coating provides modest nuclease protection. Combined coating with K₂₀ and PEI-PEG 50 provides significant nuclease protection as perceived from the strong DNA band.

FIGS. 20A-20D show confocal images of cellular uptake of cy5 labeled DNA nanostructure into freshly harvested dendritic cells. (FIG. 20A) naked DNA nanostructure, (FIG. 20B) DNA nanostructure coated with K₂₀, (FIG. 20C) DNA nanostructure coated with only PEI-PEG 50 and (FIG. 20D) DNA nanostructure coated with both K₂₀ and PEI-PEG 50. The uptake of naked DNA nanostructure and DNA nanostructure coated with K₂₀ was low. DNA nanostructure coated with only PEI-PEG 50 was rapidly degraded and ejected from cells. DNA nanostructure coated with a combination of K₂₀ and PEI-PEG 50 resulted in greater cell uptake and longer residence time in the cells.

FIG. 21A(i)-21A(vi) show schematics of one embodiment of a nucleic acid nanocapsule of the present disclosure. FIG. 21B shows schematics (top) of a 30 nm “Genghis Khan” nanostructure and a 60 nm “Bungalow” nanostructure and corresponding images (bottom) of the nanostructures in an aqueous solution. The schematic on the left shows an example of a cross-section of the wall of a nanocylinder structure (e.g., Genghis Khan or Bungalow) showing an arrangement of double helices in the structure.

FIG. 22A shows schematics of examples of single-stranded nucleic acid handles linked to a nucleic acid nanocapsule (left) and different examples of agents linked to single-stranded nucleic acid anti-handles that are complementary to the handles of the nanocapsule (right). FIG. 22B shows a schematic of an example of a nucleic acid nanostructure linked on its exterior surface to CpG oligonucleotides and scFV targeting molecules (left) and a nucleic acid nanostructure linked on its interior surface to dye and antigen (right). FIG. 22C shows schematics of different examples of agents linked to the interior surface and/or exterior surface of a nucleic acid nanocapsule of the present disclosure.

FIG. 23A shows an example of a ligation scheme for coupling PEG-NHS to PEI primary amines. The lower panels show agarose gel analysis of DNA nanostructure stability when incubated for various time lengths in serum active cell medium. C=control, 1-2-4-8-o/n time in hours. Four different protection degrees were tested: bare, N/P=0.1, N/P=1, N/P=10.

FIG. 23B shows uptake of PEI-PEG coated DNA nanostructures in bone marrow-derived dendritic cells (BMDCs) at 0 min, 30 min, 60 min, 90 min, 120 min and 150 min. The amount of uptake of PEI-PEG coated DNA nanostructures by BMDCs increases over time.

FIG. 24A shows a schematic outline of a toll-like receptor 9 (TLR9) activation experiment. FIG. 24B shows an interleukin 12 (IL12) cytokine enzyme-linked immunosorbent assay (ELISA) assay to evaluate TLR9 stimulation.

FIG. 25A shows schematics of one example of a pH-sensitive mechanism of the present disclosure. The peptide antigen cargo is linked to a nanocapsule through pH-sensitive handle/anti-handle interactions. When the nanocapsule with antigen cargo is in a low pH (e.g., pH<6) environment, the antigen cargo is release and delivered to cells. FIG. 25B shows the confocal imaging of a peptide antigen release in live cells at 15 min, 30 min, 45 min, 60 min, 75 min, 90 min, 105 min and 120 min.

FIG. 26A shows T-cell activation assay results measured by fluorescent intensity profiling with flow cytometry. FIG. 26B shows the cartoon outline of a T-cell activation experiment. FIG. 26C shows quantification of the T-cell experiment using FlowJo analysis (top, percent cell proliferation; bottom, relative amount of cell divisions).

FIGS. 27A-27F show examples of DNA cylinders. Top, cartoon representation of the target designs, where each component ring (carious shades of gray) represents an individual double helix. Top right, schematic representation of cylinder splayed open. FIGS. 27A(i), 27B(i), 27C(i), 27D(i), 27E(i) and 27F show negative-stain TEM of particles oriented axially. FIGS. 27A(ii), 27B(ii), 27C(ii), 27D(ii) and 27E(ii) show negative-stain TEM of particles oriented laterally. FIGS. 27A(i), (ii) and 27B(i), (ii) show nanostructures with a 31 nm diameter and heights of 30 nm and 65 nm, respectively. FIGS. 27C(i), (ii) and 27D(i), (ii) show nanostructures with a 60 nm diameter and heights of 30 nm and 35 nm, respectively. FIG. 27E(i), (ii) shows a nanostructure with a 87 nm diameter and height of 26 nm. FIG. 27F shows a nanostructure with a 113 nm diameter and height of 30 nm.

FIG. 28A shows a schematic of rhombic lattice points on the surface of cylinder domains of a nucleic acid nanostructure. Each point can be functionalized with a ssDNA handle. Each point has six nearest neighbors, each 8.7 nm away. Light gray points represent anti-DEC205 attachments, black points represent anti-CD40 attachments. FIG. 28B shows recombinant expression and IMAC purification of 6×His tagged anti-DEC205 scFv.

FIG. 29A shows an agarose gel assay for nuclease degradation demonstrating protection of DNA origami by hexalysine (K6) from digestion by DNAse I present in serum. 10 μL of 1 nM nanocylinders (left half of gel: −K6; right half of gel; +K6) were incubated with 10 μL of fresh cell medium (10% FBS in RPMI-1640) at 37° C. for different time points. Lanes labeled “Sample before incubation” hold DNA nanocylinder prior to treatment by serum, and therefore represents a no digestion control. FIG. 29B shows a schematic of a six-helix bundle plug for the hole in the top of dome-shaped nucleic acid nanostructures.

FIG. 30A shows a schematic of acid-pH triggered dissociation of DNA cylinder domains. Light gray strands are C-rich sequences that preferentially form dsDNA with the dark gray strands at neutral pH, but preferentially form ssDNA i-motif self-structures at pH 5.5 or lower, and therefore dissociate from the dark gray strands at acidic pH. FIG. 30B shows an agarose gel assay demonstrating dissociation of the two cylindrical domains over time at pH 5.5.

FIGS. 31A-31E show activation of BMDCs and downstream activation of OT1 CD8⁺ T cells or OT2 CD4⁺ T cells in vitro. FIG. 31A shows induction of IL-12 secretion by BMDCs after incubation with DNA-origami nanocylinders decorated with CpG danger signals on the outside or inside of the nanocylinders. Outer-surface CpG presentation is denoted by “out” while inner-surface CpG presentation is denoted by “in”, and the numbers (e.g., 100, 50, 10) indicate the final concentration of CpG in nanomolar. Induction of IL-12 was suppressed by the presence of an oligoamine shell formulated with the cylinder domains, and was only observed when CpG was displayed on the outside of the cylinders, but not when displayed on the inside of the cylinders. FIG. 31B shows induction of OT1 CD8⁺ T-cell expansion (assessed by CFSE dilution) by BMDCs treated with nanocylinders bearing OVA1 peptides at 10 nM final concentration. FIG. 31C shows OVAI peptide control. FIG. 31D shows induction of OT2 CD4⁺ T-cell expansion by BMDCs treated with nanocylinders bearing OVA2 peptides at 10 nM final concentration. FIG. 31D shows OVAII peptide control. Gray bars represent peak widths.

FIG. 32 shows that eliminating Tfr cells allows for enhanced anti-tumor B cell responses. Wild-type (WT) mice were immunized with BRAF/PTEN antigens, and 7 days later, Tfh and/or Tfr cells were sorted and transferred to Tcrα^{-/-} mice that received BRAF/PTEN tumors. Serum was analyzed 8 days later for IgG2b expression.

### DETAILED DESCRIPTION OF INVENTION

Nucleic acids (e.g., DNA) can be fabricated as three-dimensional nanostructures that are, for example, several mega-daltons in size. One such method of DNA nanostructure fabrication is referred to as DNA origami, which includes producing three-dimensional nucleic acid structures of arbitrary, predefined shape and size (see, e.g., WO 2013148186 A1). Nucleic acid nanostructures have great potential in biomedical applications, particularly because they are biodegradable, can be functionalized in a site-specific manner, and can be engineered to undergo allosteric conformational changes, allowing for precise interactions with target molecules and cells.

Practically, however, nucleic acid nanostructures have limited uses in the biomedical field due, in part, to poor structural integrity and rapid degradation under physiological conditions. Nucleic acid nanostructures typically require up to 10 mM magnesium ion (Mg²⁺) to neutralize electrostatic repulsion and thereby stabilize their shape. Thus, nucleic acid nanostructures exhibit poor structural integrity in biological buffers (e.g., buffers containing physiological levels of Mg²⁺ (e.g., 0.6 mM) and Ca²⁺ (e.g., 1.2 mM)). Additionally, the activity of DNAse I in freshly prepared cell medium containing 10% fetal bovine serum, which is typically used in biomedical applications, causes rapid degradation of nucleic acid nanostructures.

Provided herein, in various aspects and embodiments, are nucleic acid nanostructures that are engineered to maintain their structural integrity and resist nuclease degradation, even under physiological conditions of magnesium depletion and nuclease activity. Nucleic acid nanostructures herein are typically subsaturated with positively charged polyamine polymers (e.g., polylysine peptides), or a combination of polyamine polymers and PEI-PEG copolymers, which neutralize electrostatic repulsion and enhance nucleic acid resistance to nuclease degradation, thereby stabilizing the shape of the nanostructures. Without being bound by any particular theory, the primary interaction between polyamine polymers and nucleic acid nanostructures is electrostatic: positively charged polymers can weave into the nanostructures to shield the negatively charged phosphate backbone of the nucleic acids and thus promote close packing of nucleic acid helices. In some embodiments, nucleic acid nanostructures further comprise PEI-PEG copolymers. Thus, in some embodiments, nucleic acid nanostructures comprise a combination of polyamine polymers (e.g., polylysine polymer) and PEI-PEG copolymers.

Current vaccination approaches used to enhance an immune response to cancer, for example, involve isolating and activating dendritic cells ex vivo and then introducing these programmed dendritic cells back into the patient. These activated dendritic cells may home to a lymph node, present antigens to naive T-cells and stimulate and expand specific T-cell populations that elicit anti-tumor responses. Such approaches require cell isolations and in vitro dendritic-cell modifications, leading to multiple patient procedures, high cost and regulatory concerns. Greater than 90% of transplanted dendritic cells die and few home to the lymph nodes. Further, ex vivo dendritic-cell modifications may be dependent on culture conditions, and be transient, and, thus, lose effectiveness on in vivo transplantation.

Techniques for activating dendritic cells directly in vivo are limited, in part, due to a lack of agents and delivery vehicles that can withstand harsh physiological conditions that lead to degradation of the agents and delivery vehicles.

Provided herein, in some aspects, are methods for in vivo delivery to cells, such as dendritic cells, of agents loaded in (e.g., encapsulated by) carriers, referred to herein as a “pH-sensitive nucleic acid nanocapsules,” that protect the agents from the harsh physiological conditions. The interior compartment of such nanocapsules are loaded with agent(s), and the exterior nucleic acid “shell” protects the agent from low salt conditions and nuclease digestion associated with in vivo conditions. The nanocapsules, typically decorated with targeting molecules, can be directed to a particular cell type (e.g., dendritic cell). Upon entry into the cell, the nanocapsule is triggered by a change in pH to open and release the agent(s). Thus, the pH-sensitive nucleic acid nanocapsules of the present disclosure permit delivery of higher doses of agent directly to cells in vivo, relative to existing delivery technologies.

### Nucleic Acid Nanostructures

A “nucleic acid nanostructure,” as used herein, refers to nucleic acids that form (e.g., self-assemble) two-dimensional (2D) or three-dimensional (3D) shapes (e.g., reviewed in W. M. Shih, C. Lin, Curr. Opin. Struct. Biol. 20, 276 (2010), incorporated by reference herein). Nanostructures may be formed using any nucleic acid folding or hybridization methodology. One such methodology is DNA origami (see, e.g., Rothmund, P. W. K. Nature 440 (7082): 297-302 (2006), incorporated by reference herein). In a DNA origami approach, a nanostructure is produced by the folding of a longer “scaffold” nucleic acid strand through its hybridization to a plurality of shorter “staple” oligonucleotides, each of which hybridize to two or more non-contiguous regions within the scaffold strand. In some embodiments, a scaffold strand is at least 100 nucleotides in length. In some embodiments, a scaffold strand is at least 500, at least 1000, at least 2000, at least 3000, at least 4000, at least 5000, at least 6000, at least 7000, or at least 8000 nucleotides in length. The scaffold strand may be naturally or non-naturally occurring. Staple strands are typically less than 100 nucleotides in length; however, they may be longer or shorter depending on the application and depending upon the length of the scaffold strand. In some embodiments, a staple strand may be 15 to 100 nucleotides in length. In some embodiments, a staple strand is 25 to 50 nucleotides in length.

In some embodiments, a nucleic acid nanostructure may be assembled in the absence of a scaffold strand (e.g., a scaffold-free structure). For example, a number of oligonucleotides (e.g., less than 200 nucleotides or less than 100 nucleotides in length) may be assembled to form a nucleic acid nanostructure.

Other methods for assembling nucleic acid nanostructures are known in the art, any one of which may be used herein. Such methods are described by, for example, Bellot G. et al., Nature Methods, 8: 192-194 (2011); Liedl T. et al, Nature Nanotechnology, 5: 520-524 (2010); Shih W. M. et al, Curr. Opin. Struct. Biol., 20: 276-282 (2010); Ke Y. et al, J. Am. Chem. Soc, 131: 15903-08 (2009); Dietz H. et al, Science, 325: 725-30 (2009); Hogberg B. et al, J. Am. Chem. Soc, 131: 9154-55 (2009); Douglas S. M. et al, Nature, 459: 414-418 (2009); Jungmann R. et al, J. Am. Chem. Soc, 130: 10062-63 (2008); Shih W. M., Nature Materials, 7: 98-100 (2008); and Shih W. M., Nature, All: 618-21 (2004), each of which is incorporated herein by reference in its entirety.

A nucleic acid nanostructure may be assembled into one of many defined and predetermined shapes including without limitation a capsule, hemi-sphere, a cube, a cuboidal, a tetrahedron, a cylinder, a cone, an octahedron, a prism, a sphere, a pyramid, a dodecahedron, a tube, an irregular shape, and an abstract shape. The nanostructure may have a void volume (e.g., it may be partially or wholly hollow). In some embodiments, the void volume may be at least 25%, at least 50%, at least 75%, at least 85%, at least 90%, or more of the volume of the nanostructure. Thus, in some embodiments, nucleic acid nanostructures do not comprise a solid core. In some embodiments, nucleic acid nanostructures are not circular or near circular in shape. In some embodiments, nucleic acid nanostructures are not a solid core sphere. Depending on the intended use, nucleic acid nanostructures may be assembled into a shape as simple as a two-dimensional sheet or as complex as a three-dimensional capsule or lattice (or even more complex).

Nucleic acid nanostructures may be made of, or comprise, DNA, RNA, modified DNA, modified RNA, PNA, LNA or a combination thereof.

In some embodiments, nucleic acid nanostructures (e.g., nanocapsules) are rationally designed. A nucleic acid nanostructure is herein considered to be “rationally designed” if nucleic acids that form the nanostructure are selected based on pre-determined, predictable nucleotide base pairing interactions that direct nucleic acid hybridization. For example, nucleic acid nanostructures may be designed prior to their synthesis, and their size, shape, complexity and modification may be prescribed and controlled using certain select nucleotides (e.g., oligonucleotides) in the synthesis process. The location of each nucleic acid in the structure may be known and provided for before synthesizing a nanostructure of a particular shape. The fundamental principle for designing, for example, self-assembled nucleic acid nanostructures is that sequence complementarity in nucleic acid strands is selected such that, by pairing up complementary segments, the nucleic acid strands self-organize into a predefined nanostructure under appropriate physical conditions. Thus, in some embodiments, nucleic acid nanostructures are self-assembling. Similarly, handles and anti-handle nucleic acids (e.g., those linked to agents or targeting molecules) may be rationally designed to attach specifically to an interior or exterior surface of a nanostructure, in some embodiments, without intercalation or hybridization with nucleic acids forming the body of the nanostructure.

Examples of nucleic acid nanostructures for use in accordance with the present disclosure include, without limitation, capsules, lattices (E. Winfree, et al. Nature 394, 539 (1998); H. Yan, et al. Science 301, 1882 (2003); H. Yan, et al. Proc. Natl. Acad. of Sci. USA 100, 8103 (2003); D. Liu, et al. J. Am. Chem. Soc. 126, 2324 (2004); P. W. K. Rothemund, et al. PLoS Biology 2, 2041 (2004)), ribbons (S. H. Park, et al. Nano Lett. 5, 729 (2005); P. Yin, et al. Science 321, 824 (2008)), tubes (H. Yan Science (2003); P. Yin (2008)), finite two-dimensional (2D) and three dimensional (3D) objects with defined shapes (J. Chen, N. C. Seeman, Nature 350, 631 (1991); P. W. K. Rothemund, Nature 440, 297 (2006); Y. He, et al. Nature 452, 198 (2008); Y. Ke, et al. Nano. Lett. 9, 2445 (2009); S. M. Douglas, et al. Nature 459, 414 (2009); H. Dietz, et al. Science 325, 725 (2009); E. S. Andersen, et al. Nature 459, 73 (2009); T. Liedl, et al. Nature Nanotech. 5, 520 (2010); D. Han, et al. Science 332, 342 (2011)), and macroscopic crystals (J. P. Meng, et al. Nature 461, 74 (2009)). Other nucleic acid nanostructures (e.g., nanocapsules) may be used as provided herein.

Polylysine, a cationic polymer, is known to be efficient in condensing plasmid DNA into compact particles, for example, for delivery of therapeutic DNA. DNA is a highly negatively charged polymer due to the repeating phosphate groups along the polymer backbone. The interaction with cationic polymers such as polylysine is therefore an electrostatic one. It is generally accepted that DNA condensation occurs through neutralization of negative charges on the DNA by its interactions with cationic polylysine polymers, followed by hydrophobic collapse as water is displaced from the DNA structure. Generally, DNA is super-saturated with polylysine polymers such that most or all of the negative charges of the DNA are neutralized, and the DNA condenses into a compact particle of 12 nm to 300 nm in diameter, depending on the weight of the polylysine polymer and the condensation conditions (e.g., charge ratio between polymer and DNA, salt concentration and temperature). In some embodiments, the term “condensed nucleic acid” refers to a nucleic acid particle that has a diameter and/or volume that is less than 80%, less than 70%, less than 60%, less than 50%, or less than 40% of the diameter and/or volume of its non-condensed state (e.g., without being saturated or supersaturated with polylysine). Unlike the condensed, compacted DNA particles described above, the nucleic acid nanostructures of the present disclosure are not condensed into compact particles when complexed with polyamine polymers in accordance with the present disclosure. Rather, nucleic nanostructures provided herein are “subsaturated” with polyamine polymers such that the architecture of the structures is not compromised. That is, nucleic acid nanostructures of the present disclosure have a 2D or 3D shape, despite the additional weight of and interactions with positively-charged polyamine polymers.

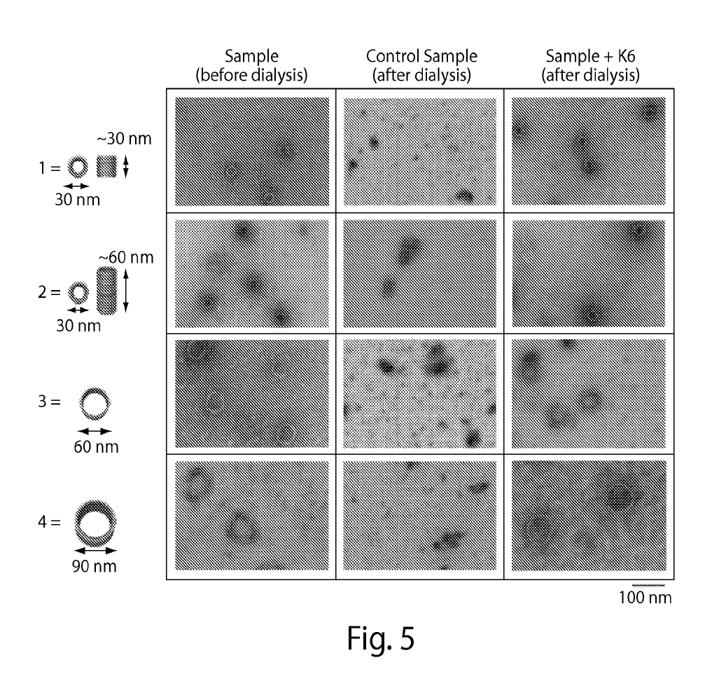

Thus, nucleic acid nanostructures provided herein, in some embodiments, are subsaturated with polyamine polymers, or a combination of polyamine polymers and PEI-PEG copolymers. As discussed above, nucleic acid nanostructures are considered to be “subsaturated” with polyamine polymers and/or PEI-PEG copolymers if less than 100% of the phosphates of the nucleic acid nanostructure backbone are linked to amines of polyamine polymers and/or amines of PEI-PEG copolymers. In some embodiments, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15% or less than 10% of the phosphates of nucleic acid nanostructure are linked to amines of the polyamine polymers. In some embodiments, 10% to 90%, 10% to 80%, 10% to 50%, 20% to 90%, or 20% to 80% of the phosphates of the nucleic acid nanostructure backbone are linked to amines of polyamine polymers and/or amines of PEI-PEG copolymers. At such subsaturated levels, nucleic acid nanostructures still maintain their structural integrity (e.g., keep their original shape), despite their interactions with polyamine polymers and/or PEI-PEG copolymers. It should be understood that a nucleic acid nanostructure subsaturated with polyamine polymers and/or PEI-PEG copolymers is herein considered to “maintain its structural integrity” if the shape of the nanostructure, under the same environmental conditions, can be distinguished/discerned for a period of time that is greater than that of a control nucleic acid nanostructure (e.g., a similar nucleic acid nanostructure that is not subsaturated with polyamine polymers and/or PEI-PEG copolymers). For example, as shown in FIG. 5, nucleic acid nanostructures subsaturated with polylysine homopolymers can be distinguished as circular shapes (right hand column), while control structures without polylysine homopolymers appear as irregular masses (center column). Thus, the nucleic acid nanostructures subsaturated with polylysine homopolymers of FIG. 5 are considered to have maintained their structural integrity.

The relationship between amines of polyamine polymers, and/or amines of PEI-PEG copolymers, and phosphates of nucleic acid nanostructures may also be described in terms of an amine to phosphate ratio. The “N/P ratio,” herein, refers is the ratio of positive (+) charges contributed to a structure by a primary, secondary or tertiary amine that can be protonated (e.g., in the side chain of a peptide) to and negative (−) charges contributed to a structure by phosphates of a nucleic acid backbone. For example, lysine in the middle of a peptide contributes 1+charge, while lysine at the N-terminus of a peptide contributes 2+charges. Thus, “subsaturated,” refers to a N:P ratio of 0.9:1 or lower (i.e., lower number of amines compared to phosphates). “Saturated,” by comparison, refers to a N:P ratio of 1:1. “Supersaturated” refers to a N:P ratio of 1.1:1 or greater (i.e., greater number of amines compared to phosphates). Thus, in some embodiments, the ratio of amines or amines to phosphate (e.g., amines of polyamine polymers, or amines of PEI-PEG copolymers that interact with (e.g., are linked to) phosphates of a nucleic acid nanostructure backbone) is lower than 1:1. For example, the ratio of amines phosphates may be 0.9:1, 0.8:1, 0.7:1, 0.6:1, 0.5:1, 0.4:1, 0.3:1, 0.2:1 or 0.1:1. In some embodiments, the ratio of amines to phosphates is 0.9:1 to 0.1:1, 0.9:1 to 5:1, 0.8:1 to 0.1:1 or 0.5:1 to 0.1:1.

As used herein, the terms “nucleic acid” and/or “oligonucleotide” may refer to at least two nucleotides covalently linked together. A nucleic acid of the present disclosure may generally contain phosphodiester bonds, although in some cases, nucleic acid analogs are included that may have other backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. Nucleic acid may have a homogenous backbone (e.g., entirely phosphodiester or entirely phosphorothioate) or a heterogeneous (or chimeric) backbone. Phosphorothioate backbone modifications render a nucleic acid less susceptible to nucleases and thus more stable (as compared to a native phosphodiester backbone nucleic acid) under certain conditions. Other linkages that may provide more stability to a nucleic acid include without limitation phosphorodithioate linkages, methylphosphonate linkages, methylphosphorothioate linkages, boranophosphonate linkages, peptide linkages, alkyl linkages, dephospho type linkages, and the like. Thus, in some instances, nucleic acids have non-naturally occurring backbones. Modifications of the ribose-phosphate backbone may be done, for example, to facilitate the addition of labels, or to increase the stability and half-life of such molecules in physiological environments.

Nucleic acids may be single-stranded (ss) or double-stranded (ds), as specified, or may contain portions of both single-stranded and double-stranded sequence (e.g., are partially double-stranded). Nucleic acids may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, and isoguanine. As used herein, the term “nucleoside” includes nucleotides as well as nucleoside and nucleotide analogs, and modified nucleosides such as amino modified nucleosides. In addition, “nucleoside” includes non-naturally occurring analog structures. Thus, for example, the individual units of a peptide nucleic acid, each containing a base, are referred to herein as a nucleoside.

Nucleic acids include DNA such as B-form DNA, D-form DNA and L-form DNA and RNA, as well as various modifications thereof. Modifications include base modifications, sugar modifications, and backbone modifications. Non-limiting examples of these are provided below.

Non-limiting examples of DNA variants that may be used as provided herein are L-DNA (the backbone enantiomer of DNA, known in the literature), peptide nucleic acids (PNA) bisPNA clamp, a pseudocomplementary PNA, a locked nucleic acid (LNA), or co-nucleic acids of the above such as DNA-LNA co-nucleic acids. It is to be understood that nucleic acids used as provided herein may be homogeneous or heterogeneous in nature. As an example, they may be completely DNA in nature or they may comprise DNA and non-DNA (e.g., LNA) monomers or sequences. Thus, any combination of nucleic acid elements may be used. The nucleic acid modification may render the nucleic acid more stable and/or less susceptible to degradation under certain conditions. For example, in some instances, the nucleic acids are nuclease-resistant.

Methods of synthesizing nucleic acids (e.g., ssDNA or dsDNA, or ssRNA or dsRNA) are known in the art and are described, for example, in U.S. Pat. Nos. 5,143,854 and 5,445,934, herein incorporated in their entirety.

Nucleic acids may be synthesized in vitro. Methods for synthesizing nucleic acids, including automated nucleic acid synthesis, are also known in the art. Nucleic acids having modified backbones, such as backbones comprising phosphorothioate linkages, and including those comprising chimeric modified backbones may be synthesized using automated techniques employing either phosphoramidate or H-phosphonate chemistries. (F. E. Eckstein, “Oligonucleotides and Analogues—A Practical Approach” IRL Press, Oxford, UK, 1991, and M. D. Matteucci and M. H. Caruthers, Tetrahedron Lett. 21, 719 (1980)) Aryl- and alkyl-phosphonate linkages can be made, e.g., as described in U.S. Pat. No. 4,469,863; and alkylphosphotriester linkages (in which the charged oxygen moiety is alkylated), e.g., as described in U.S. Pat. No. 5,023,243 and European Patent No. 092,574, can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165; Crooke S T et al. (1996) Annu Rev Pharmacol Toxicol 36:107-129; and Hunziker J et al. (1995) Mod Synth Methods 7:331-417.

Nucleic acids may additionally or alternatively comprise modifications in their sugars. For example, a β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, arabinose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

Nucleic acids may comprise modifications in their bases. Modified bases include modified cytosines (such as 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil), modified guanines such as 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. The nucleic acids may comprise universal bases (e.g. 3-nitropyrrole, P-base, 4-methyl-indole, 5-nitro-indole, and K-base) and/or aromatic ring systems (e.g. fluorobenzene, difluorobenzene, benzimidazole or dichloro-benzimidazole, 1-methyl-1H-[1,2,4]triazole-3-carboxylic acid amide). A particular base pair that may be incorporated into the oligonucleotides of the invention is a dZ and dP non-standard nucleobase pair reported by Yang et al. NAR, 2006, 34(21):6095-6101. dZ, the pyrimidine analog, is 6-amino-5-nitro-3-(1′-β-D-2′-deoxyribofuranosyl)-2(1H)-pyridone, and its Watson-Crick complement dP, the purine analog, is 2-amino-8-(1′-β-D-1′-deoxyribofuranosyl)-imidazo[1,2-a]-1,3,5-triazin-4(8H)-one.

In exemplary embodiments, nucleic acid nanostructures comprise single-stranded genomic DNA. For example, nucleic acid nanostructures may comprise linear or circular single-stranded M13 plasmid DNA. In some embodiments, nucleic acid nanostructures do not comprise plasmid DNA.

It should be appreciated that nucleic acid nanostructures of the present disclosure, in some embodiments, do not include condensed nucleic acid. As used herein, “condensed nucleic acid” refers to compacted nucleic acid, for example, that is twisted and coiled upon itself (see, e.g., Teif V B, et al. Progress in Biophysics and Molecular Biology 105 (3): 208-222, incorporated by reference herein). The term “condensed nucleic acid” excludes nucleic acid nanostructures that have a distinct 2D or 3D architecture.

It should also be appreciated that nucleic acid nanostructures of the present disclosure, in some embodiments, do not include coding nucleic acid. That is, in some embodiments, nucleic acid nanostructures comprise non-coding nucleic acids (e.g., nucleic acids that do not encode proteins). As used herein, a “coding nucleic acid” refers to a nucleic acid containing a nucleotide sequence that specifies a sequence of amino acids of a protein (e.g., a therapeutic protein). Thus, a “non-coding nucleic acid” is a nucleic acid that does not specify a sequence of amino acids of a protein and, accordingly, is not transcribed into RNA or translated into protein. In other embodiments, it should be understood that a nucleic acid nanostructure may contain one or more coding nucleic acids.

In some embodiments, nucleic acids used to make nucleic acid nanostructures do not code for any amino acid. In some embodiments, nucleic acids used to make nucleic acid nanostructures do not code for more than 1, 2, 3, 4 or 5 consecutive amino acids.

In some embodiments, nucleic acids used to make nucleic acid nanostructures do not include art-recognized regulatory elements/sequences such as promoters, enhancers, polyA sequences and/or ribosomal binding site sequences.

In some embodiments, nucleic acids used to make nucleic acid nanostructures are not plasmids.

In some embodiments, nucleic acids used to make nucleic acid nanostructures contain more than one nucleic acid, and the nucleic acid are different from each other. That is, the nucleic acids of a nucleic acid nanostructure may comprise a plurality of different nucleic acids.

In some embodiments, nucleic acid nanostructures are not encapsulated by or coated with (e.g., linked to) lipids. For example, a variety of gene delivery methods of the prior art make use of nucleic acid nanostructures that are linked to hydrophobic moieties and/or covered by lipids (e.g., such as a lipid bilayer), which function to prevent nuclease degradation (see, e.g., WO 2013148186 A1). The present disclosure, in some embodiments, excludes nucleic acid nanostructures that are linked to hydrophobic moieties and/or covered by lipids. In other embodiments, however, a nucleic acid nanostructure may contain one or more nucleic acids linked to one or more hydrophobic moieties and/or lipids.

### Nucleic Acid Nanocapsules

A “nucleic acid nanocapsule” refers to a nucleic acid nanostructure having an exterior surface, referred to as a “shell,” and an interior compartment for encapsulation of an agent. A “nucleic acid nanostructure,” more generally, refers to nucleic acids that form (e.g., self-assemble) two-dimensional (2D) or three-dimensional (3D) shapes (e.g., reviewed in W. M. Shih, C. Lin, Curr. Opin. Struct. Biol. 20, 276 (2010)), as described above. An agent is considered to be “encapsulated” by a nucleic acid nanocapsule if the agent is within the compartment of the nanocapsule, either linked directly or indirectly to an interior surface of the nanoparticle, or otherwise contained within the compartment. That is, agents, in some embodiments, are encapsulated by a nucleic acid nanostructure, rather than being intercalated with nucleic acids that form the nanostructure. For example, with respect to nanocapsule architectures, agents may be attached (e.g., via a handle/anti-handle configuration) to the interior surface of the nanocapsule, rather than being intercalated into nucleic acid “walls” of a nanocapsule. Intercalation refers to the insertion of an agent, non-covalently, between planar bases of DNA. Agents intercalated with nucleic acids that form a nanostructure are typically exposed, rather than protected, from the surrounding environment and, thus, are more prone to degradation.

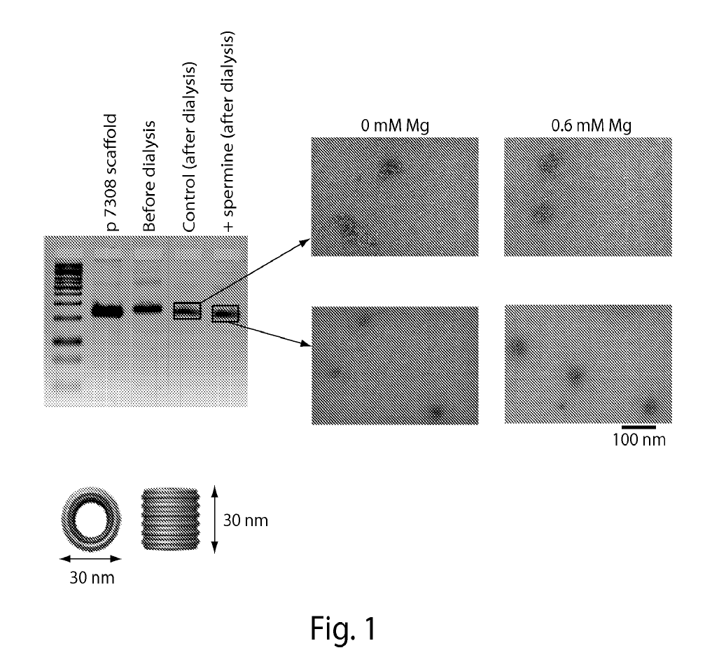

Nucleic acid nanocapsules may be “capped” at each end so as to form a capsule-like structure having an interior compartment. Thus, in some embodiments, at each end of a nucleic acid capsule there is an opening having a diameter of less than 20 nm. In some embodiments, at each end of a nucleic acid capsule there is an opening having a diameter of less than 15 nm, less than 10 nm, less than 9 nm, less than 8 nm, less than 7 nm, less than 6 nm, less than 5 nm, less than 4 nm, or less than 3 nm in diameter. In some embodiments, at each end of a nucleic acid capsule there is an opening having a diameter of 2 nm to 20 nm, 2 nm to 15 nm, or 2 nm to 10 nm. In some embodiments, each end of a nanocapsule is capped with a nucleic acid nanostructure formed from concentric rings, as shown in FIG. 1A. In the exemplary nanocapsule shown in FIG. 1A, there still remains a small opening (e.g., less than 10 nm in diameter) at each end of nanocapsule, even after capping the nanocapsule with the concentric rings. This is a result of a restraint on the degree to which nucleic acids can be bent. In some embodiments, this small opening is closed, or “plugged,” with nucleic acids nanostructures having a diameter (or dimension) of less than 10 nm (e.g., less than 9 nm, less than 8 nm, less than 7 nm, less than 6 nm, less than 5 nm, less than 4 nm, less than 3 nm or less than 2 nm). For example, the open ends of nucleic acid nanocapsules may be closed with one or more bundle(s) of nucleic acid helices. For example, the open ends of nucleic acid nanocapsules may be closed with one or more n-helix bundle(s), where n is 1, 2, 3, 4, 5, 6, 7, 8, 9 or 10. The present disclosure contemplates other means of closing open ends of a nucleic acid nanocapsule.

In some embodiments, a nucleic acid nanocapsule may be assembled in the absence of a scaffold strand (e.g., a scaffold-free structure). For example, a number of short nucleic acids (e.g., less than 200 nucleotides or less than 100 nucleotides in length) may be assembled to form a nucleic acid nanocapsule.

One advantage of nucleic acid nanostructures is that they can be rationally designed to assemble in a precise and controlled manner into one of many defined and predetermined shapes including without limitation a hemi-sphere, a cube, a cuboidal, a tetrahedron, a cylinder, a cone, an octahedron, a prism, a sphere, a pyramid, a dodecahedron, a tube, an irregular shape, and an abstract shape. Thus, while for simplicity the term “capsule” is used herein to describe the nanostructures, it should be understood that a nanocapsule of the present disclosure may be any shape, not only spherical or oblong as shown in FIG. 1A, as long as the structure contains an exterior surface and an interior compartment. That is, the nanocapsules of the present disclosure have a void volume (e.g., are partially or wholly hollow). Thus, nucleic acid nanocapsules do not comprise a solid core. In some embodiments, the void volume may be at least 25%, at least 50%, at least 75%, at least 85%, at least 90%, or more of the volume of the nanocapsule.

A nucleic acid nanocapsule of the present disclosure may be as small as 5 nanometers (nm) in diameter (or width, height, or length, depending on the shape of the capsule), and as large as 10 micrometers (μm). Thus, the term “nanocapsule” encompasses micrometer-sized capsules, or “microcapsules,” having a diameter of up to 10 μm. In some embodiments, a nanocapsule of the present disclosure has a diameter of 5 nm to 1 μm, 5 nm to 900 nm, 5 nm 10 to 800 nm, 5 nm to 700 nm, 5 nm to 600 nm, 5 nm to 500 nm, 5 nm to 400 nm, 5 nm to 300 nm, 5 nm to 200 nm, 5 nm to 150 nm, 5 nm to 100 nm, 5 nm to 90 nm, 5 nm to 80 nm, 5 nm to 70 nm, 5 nm to 60 nm, 5 nm to 50 nm, 5 nm to 40 nm, or 5 nm to 30 nm. In some embodiments, a nanocapsule of the present disclosure has a diameter of 10 nm to 100 nm, 20 nm to 90 nm, 30 nm to 80 nm, 40 nm to 70 nm. In some embodiments, a nanocapsule of the present disclosure has a diameter of 60 nm.

In some embodiments, nucleic acid nanocapsules are formed in the shape of a capsule, as shown in FIG. 1A. For example, a nanocapsule may be formed by the assembly of multiple nanostructures joined to form interfaces between the nanostructures. In some embodiments, a nanocapsule contains 2, 3, 4 or more nanostructures, joined to each other to form multiple interfaces. FIG. 1A depicts an exemplary embodiment of the present disclosure in which two cylindrical-shaped (also referred to as barrel-shaped) nanostructures are joined together, and then each end of the capsule is “capped” with concentric rings of nucleic acid in the form of “caps.” The height (along the y-axis) of each cylindrical-shaped nanostructure and/or cap-shaped nanostructure, in some embodiments, is 10 nm to 100 nm, 20 nm to 90 nm, 30 nm to 80 nm, 40 nm to 70 nm, or 50 nm to 60 nm. In some embodiments, the height of each cylindrical-shaped nanostructure is 20 nm, 30 nm, 40 nm, 50 nm, 60 nm, 70 nm or 80 nm. The diameter, or width, (along the x-axis) of each cylindrical-shaped nanostructure and/or cap-shaped nanostructure, in some embodiments, is 10 nm to 100 nm, 20 nm to 90 nm, 30 nm to 80 nm, 40 nm to 70 nm, or 50 nm to 60 nm. In some embodiments, the diameter of each cylindrical-shaped nanostructure is 20 nm, 30 nm, 40 nm, 50 nm, 60 nm, 70 nm or 80 nm. In some embodiments, the total height of the nanocapsule, for example, when oriented as shown in FIG. 1A, is 10 nm to 500 nm, 10 nm to 400 nm, 10 nm to 300 nm, 10 nm to 200 nm, 10 nm to 150 nm, 10 nm to 100 nm, 10 nm to 90 nm, 10 nm to 80 nm, 10 nm to 10 nm, or 10 nm to 10 nm. In some embodiments, the total height of the nanocapsule is 50 nm, 60 nm, 70 nm, 80 nm, 90 nm, 100 nm, 110 nm, 120 nm, 130 nm, 140 nm or 150 nm.

In some embodiments, nucleic acid nanocapsules comprise single-stranded genomic DNA. For example, in some embodiments, nucleic acid nanocapsules comprise single-stranded genomic DNA that self-assembles, guided by short nucleic acid “staple” strands, to form a nucleic acid nanostructure. For example, nucleic acid nanostructures that form a nucleic acid nanocapsule may comprise linear or circular single-stranded M13 plasmid DNA. In some embodiments, nucleic acid nanocapsules do not comprise plasmid DNA.

### Protective Coatings

Aspects of the present disclosure provide nucleic acid nanocapsules “coated” with polyamine polymers (e.g., polylysine polymers) and/or copolymers of cationic poly(ethylene imine) and polyethylene glycol (referred to as “PEI-PEG copolymers”) that protect the nanocapsules from degradation, for example, under physiological conditions of magnesium and/or calcium depletion and nuclease activity. Nucleic acid nanostructures, in general, typically require up to 10 mM magnesium ion (Mg2⁺) to neutralize electrostatic repulsion and thereby stabilize their shape. Thus, such structures exhibit poor structural integrity in biological buffers (e.g., buffers containing physiological levels of Mg2⁺ (e.g., 0.6 mM) and Ca2⁺ (e.g., 1.2 mM)). Further, the activity of DNAse I in freshly prepared cell medium containing 10% fetal bovine serum, which is typically used in biomedical applications, causes rapid degradation of nucleic acid nanostructures. The structural integrity of nucleic acid nanocapsules can be maintained, even under physiological conditions (e.g., including low salt conditions), by linking the nanocapsules to positively charged polyamine polymers (e.g., polylysine peptides) and/or PEI-PEG copolymers, which neutralize electrostatic repulsion and enhance nucleic acid resistance to nuclease degradation, thereby stabilizing the shape of the nanocapsules.

For simplicity, nucleic acid nanocapsules subsaturated with polyamine polymers and/or PEI-PEG copolymers may be referred to as being “coated” with polyamine polymers and/or PEI-PEG copolymers. In some embodiments, nucleic acid nanocapsules are coated with polyamine (e.g., polylysine) polymers. In some embodiments, nucleic acids nanocapsules are coated with PEI-PEG copolymers. In some embodiments, nucleic acid nanocapsules are coated with a combination of polyamine (e.g., polylysine) polymers and PEI-PEG copolymers.

In some embodiments, nucleic acid nanocapsules are not coated with (e.g., linked to) polyamine polymers and/or PEI-PEG copolymer. In some embodiments, nucleic acid nanocapsules are linked to hydrophobic moieties and/or are covered by lipids (e.g., such as a lipid bilayer), which function to prevent nuclease degradation (see, e.g., WO 2013148186 A1). In some embodiments, the present disclosure excludes nucleic acid nanocapsules that are linked to hydrophobic moieties and/or covered by lipids.

### Polyamine Polymers

Polyamine polymers of the present invention are generally cationic polymers, which, without being bound by any particular theory, may be used to shield the negatively charged phosphate backbone of nucleic acids, thereby promoting close packing of nucleic acid helices to stabilize the shape of and slow down nuclease degradation of the nanostructures. A “polyamine polymer,” as used herein, encompasses compounds having two or more primary amine groups. Polyamine polymers also encompass compounds that have secondary (e.g., R₂NH) and tertiary (e.g., R₃N) amines. Secondary and tertiary amines may be protonated in a similar manner to primary amines and interact electrostatically with phosphates in nucleic acid backbones. Polyamine polymers also encompass polycationic polymers. Polyamine polymers, in some embodiments, comprise cations that are present, in some embodiments, at regularly spaced intervals.

As cations, polyamine polymers bind to nucleic acids through electrostatic (e.g., non-covalent) interactions. Thus, in some embodiments, polyamine polymers as provided herein are non-covalently linked (e.g., via electrostatic interactions) to nucleic acid nanostructures. In other embodiments, however, polyamine polymers may be covalently linked to nucleic acid nanostructures (see, e.g., Eskelinen et. al. small 2012, 8(13):2016-2020, incorporated by reference herein).

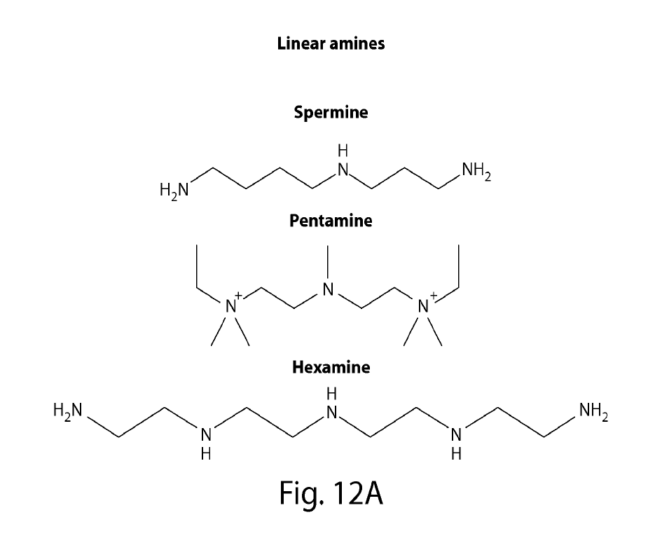

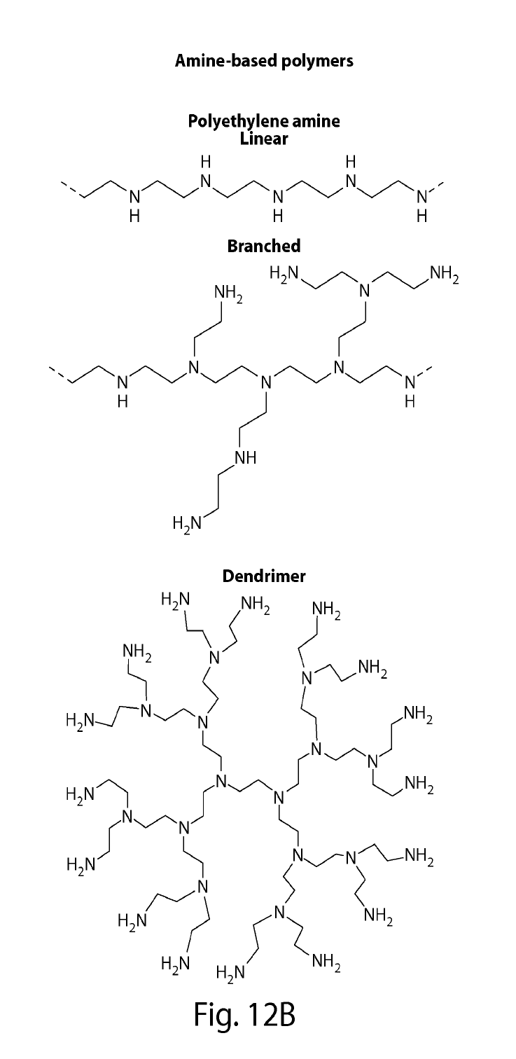

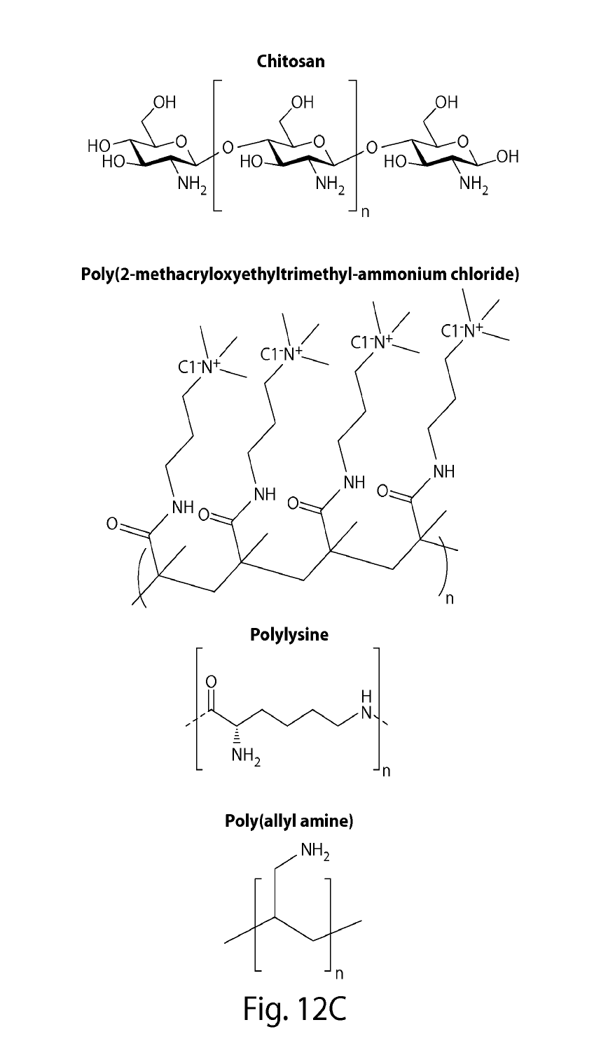

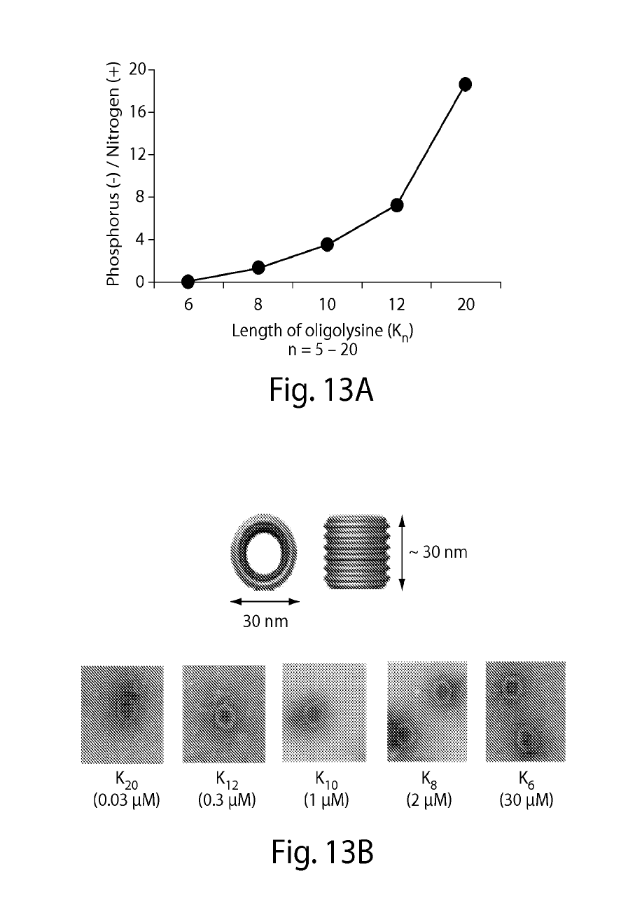

Polyamine polymers may comprise any one or more functional groups in addition to its primary amine groups. As used herein, a “functional group” refers to an atom or group of atoms, such as a carboxyl group, that replaces hydrogen in an organic compound and determines the chemical behavior of the compound. Examples of common functional groups include, without limitation, alkane, ketone, alkene, aldehyde, alkyne, imine, carboxylic acid, alkyl halide, ester, alcohol, thioester, thiol, amide, acyl phosphate, acid chloride, thioether, phosphate monoester, phenol and phosphate diester. Polyamine polymers of the present disclosure include linear (e.g., spermine, pentamine, hexamine), branched and dendrimer polymers, non-limiting examples of which are depicted in FIGS. 12A-12C. Polyamine polymers as provided herein also include chitosan, poly(2-methacryloxyethyltrimethyl-ammonium chloride) and poly(allyl amine) (see, e.g., FIG. 12C). Other non-limiting examples of polyamine polymers for use as provided herein are shown in Table I. Polyamine polymers of the present disclosure, in some embodiments, are not limited by length of the polymer. For example, in some embodiments, polyamine polymers comprise at least 4 functional groups or monomers or units. In some embodiments, polyamine polymers comprise at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 functional groups or monomers or units. In some embodiments, polyamine polymers comprise 4, 5, 6, 7, 8, 9, 10, 11, 12, 13, 14, 15, 16, 17, 18, 19, 20, 21, 22, 23, 24, 25, 26, 27, 28, 29, 30 or more functional groups or monomers or units. In some embodiments, polyamine polymers comprise 4 to 150, 4 to 100, 4 to 50, 4 to 25, 6 to 150, 6 to 100, 6 to 50, 6 to 25, 8 to 150, 8 to 100, 8 to 50, 8 to 25, 10 to 150, 10 to 100, 10 to 50, 10 to 25, 12 to 150, 12 to 100, 12 to 50 or 12 to 25 functional groups or monomers or units. If the polymer is a peptide, the monomer or unit may be an amino acid. The terms “unit,” “subunit” and “monomer” may be used interchangeably. An example of a monomer of a polymer is an amino acid of a peptide or protein.

TABLE I

| Peptide sequence | Notes |
|---|---|
| Kn | 6 ≦ n ≧ 100; K = Lysine |
| Rn | 6 ≦ n ≧ 100; R = Arginine |
| Hn | 6 ≦ n ≧ 100; H = Histidine |
| (KXKX)n | 1 ≦ n ≧ 20; K = lysine, X = R (Arginine), G (Glycine), P |
| | (Proline), H (Histidine) |
| (KXYKXY)n | 1 ≦ n ≧ 20; K = lysine, X and Y = R (Arginine), G |
| | (Glycine), A (Alanine) |
| (KXYZKXYZ)n | 1 ≦ n ≧ 20; K = lysine, X, Y and Z = R (Arginine), G |
| | (Glycine), A (Alanine) |
| (KXYZAKXYZA)n | 1 ≦ n ≧ 20; K = lysine, X, Y, Z and A = R (Arginine), G |
| | (Glycine), A (Alanine) |
| Kn [Peptide | The oligolysine domain may be attached to a peptide that |
| sequence that is | is known to inhibit nuclease activity. 6 ≦ n ≧ 100; |
| known to show | Example peptide sequence are: CD14 = WSRISLSR |
| nuclease inhibition] | (SEQ ID NO: 4); CD18 = YFKWSRISLSRY (SEQ ID NO: 5); |
| | LL37 = LLGDFFRKSKEKIGKEFKRIVQRIKDFLRNLVPRTES |
| | (SEQ ID NO: 6) |

In some embodiments, polyamine polymers are branched, as shown, for example, in FIGS. 12A-12C.

Aspects of the present disclosure relate to polyamine polymers that comprise amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine) and/or analogs thereof. Thus, polyamine polymers may comprise or consist of peptides (e.g., short chains of amino acid monomers linked by peptide (e.g., amide) bonds). In some embodiments, polyamine polymers comprise positively charged amino acids such as lysine and/or arginine. In some embodiments, polyamine polymers comprise histidine. Amino acid-based polyamine polymers may be homopolymers, which may comprise a plurality of contiguous identical amino acids such as, for example, a chain of contiguous lysine amino acids (e.g., polylysine), a chain of contiguous arginine amino acids (e.g., polyarginine), or a chain of contiguous histidine amino acids (e.g., polyhistidine). Thus, in some embodiments, provided herein are nucleic acid nanostructures that comprise (e.g., are subsaturated) with polylysine polymers (e.g., KKK(K)ₙ, where n≧1). In some embodiments, the polylysine polymers may be poly-L-lysine polymers. In other embodiments, provided herein are nucleic acid nanostructures that comprise (e.g., are subsaturated) with polyarginine polymers (e.g., RRR(R)ₙ, where n≧1). In other embodiments, provided herein are nucleic acid nanostructures that comprise (e.g., are subsaturated) with polyhistidine polymers (e.g., HHH(H)ₙ, where n≧1). In some embodiments, nucleic acid nanostructures do not comprise a histidine tag.

As discussed above, polyamine polymers of the present disclosure, in some embodiments, are not limited by length of the polymer. Thus, in some embodiments, amino-acid based polymers comprise at least 4 amino acids. In some embodiments, polyamine polymers comprise at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 amino acids. In some embodiments, polyamine polymers comprise 4, 5, 6, 7, 8, 9, 10, 11, 12, 13, 14, 15, 16, 17, 18, 19, 20, 21, 22, 23, 24, 25, 26, 27, 28, 29, 30 or more amino acids. In some embodiments, polyamine polymers comprise 4 to 150, 4 to 100, 4 to 50, 4 to 25, 4 to 15, 6 to 150, 6 to 100, 6 to 50, 6 to 25, 6 to 15, 8 to 150, 8 to 100, 8 to 50, 8 to 25, 8 to 15, 10 to 150, 10 to 100, 10 to 50, 10 to 25, 12 to 150, 12 to 100, 12 to 50 or 12 to 25 amino acids.

In some embodiments, polyamine polymers comprise or consist of peptides. The percent composition of peptides, in some embodiments, is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% lysine. In some embodiments, the percent composition of peptides is 50% to 100%, 55% to 100%, 60% to 100%, 65% to 100%, 70% to 100%, 75% to 100%, 80% to 100%, 85% to 100% or 90% to 100% lysine.

Lysines of polyamine polymers, in some embodiments, are separated from each other by at least 1, at least 2, at least 3, at least 4, at least 5, at least 6, at least 7, at least 8, at least 9, at least 10 or more, non-amine containing amino acids such as non-lysine amino acids. In some embodiments, lysines of polyamine polymers are separated from each other by 1 to 5, or 1 to 10, non-amine containing amino acids such as non-lysine amino acids. In some embodiments, lysines of polyamine polymers are regularly spaced. The following are non-limiting examples of linear polyamine polymers having regularly-spaced lysine (K), where X is a non-lysine amino acid, or functional group, and n is any integer equal to or greater than 1:
(i) K-X-(K-X-)ₙ-K, or X-(K-X-)ₙ, K-X-(K-X-)ₙ, or X-(K-X-)ₙ-K;
(ii) K-X-X-(K-X-X-)ₙ-K, or X-X-(K-X-X-)ₙ, or K-X-X-(K-X-X-)ₙ, or X-X-(K-X-X-)ₙ-K; or
(iii) K-X-X-X-(K-X-X-X-)ₙ-K, or X-X-X-(K-X-X-X)ₙ, or K-X-X-X-(K-X-X-X-)ₙ,
   or
X-X-X-(K-X-X-X)ₙ-K.

In some embodiments, polyamine polymers are branched.

### PEI-PEG Polymers

Copolymers of cationic poly(ethylene imine) (PEI) and polyethylene glycol (PEG) are contemplated herein. PEI-PEG copolymers, in combination with polyamine polymers, may be used, in some embodiments, to shield the negatively charged phosphate backbone of nucleic acids, thereby promoting close packing of nucleic acid helices to stabilize the shape of and slow down nuclease degradation of the nanostructures. As cations, PEG-PEI copolymers bind to nucleic acids through electrostatic (e.g., non-covalent) interactions. Thus, in some embodiments, PEG-PEI copolymers as provided herein are non-covalently linked (e.g., via electrostatic interactions) to nucleic acid nanostructures. In other embodiments, however, PEG-PEI copolymers may be covalently linked to nucleic acid nanostructures.

In some embodiments, PEI-PEG copolymers are synthesized by reacting N-hydroxysuccinimide (NHS)-activated PEG with PEI. In some embodiments, the molar ratio of PEI to PEG in a PEI-PEG copolymer of the present disclosure is 1:5 (˜50 primary amines) to 1:50 (˜0 primary amines). For example, the molar ratio of PEI to PEG may be 1:5, 1:10, 1:15, 1:20, 1:25 (˜25 primary amines), 1:30, 1:35, 1:40, 1:45 or 1:50. In some embodiments, a PEI-PEG copolymer has a molar ratio of PEI to PEG of 1:50.

In some embodiments, nucleic acid nanostructures of the present disclosure are subsaturated with PEI-PEG copolymers (e.g., subsaturated with a combination of PEI-PEG copolymers and polyamine polymers). Nucleic acid nanostructures are considered to be “subsaturated” with PEI-PEG copolymers if less than 100% of the phosphates of the nucleic acid nanostructure backbone are linked to amines of PEI-PEG copolymers. In some embodiments, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15% or less than 10% of the phosphates of nucleic acid nanostructure are linked to amines of the PEI-PEG copolymers. In some embodiments, 10% to 90%, 10% to 80%, 10% to 50%, 20% to 90%, or 20% to 80% of the phosphates of the nucleic acid nanostructure backbone are linked to amines of PEI-PEG copolymers. At such subsaturated levels, nucleic acid nanostructures still maintain their structural integrity (e.g., keep their original shape), despite their interactions with PEI-PEG copolymers. It should be understood that a nucleic acid nanostructure subsaturated with PEI-PEG copolymers is herein considered to “maintain its structural integrity” if the shape of the nanostructure, under the same environmental conditions, can be distinguished/discerned for a period of time that is greater than that of a control nucleic acid nanostructure (e.g., a similar nucleic acid nanostructure that is not subsaturated with PEI-PEG copolymers).

In some embodiments, PEI-PEG copolymers and polyamine polymers are added sequentially to a nucleic acid nanostructure. For example, in some embodiments, polyamine polymers are linked to a nucleic acid nanostructure, and then PEI-PEG copolymers are linked to the nucleic acid nanostructure. Conversely, in some embodiments, PEI-PEG copolymers are linked to a nucleic acid nanostructure, and then polyamine polymers are linked to the nucleic acid nanostructure. In some embodiments, addition of polymers and copolymers to a nucleic acid nanostructure is simultaneous. For example, a mixture of polyamine polymers and PEI-PEG copolymers may be added to a nucleic acid nanostructure.

pH-Sensitivity

Nucleic acid nanocapsules of the present disclosure are sensitive to changes in environmental pH. A nucleic acid nanocapsule is considered to be sensitive to pH if it undergoes a structural change in response to a change in pH. For example, “opening” of a nucleic acid nanocapsule as provided herein is pH-dependent. FIG. 1A depicts an illustrative example of a nucleic acid nanocapsule of the present disclosure. The nanocapsule is assembled from 2 cylindrical-shaped nucleic acid nanostructures and 2 nucleic acid “caps” (e.g., concentric rings of nucleic acid) (FIG. 1A(i)). One cylindrical-shaped nanostructure is lined with pH-sensitive single-stranded nucleic acid, referred to as “handles” (FIG. 1A(iv), top, white dotted line representing position of handles). The other cylindrical-shaped nanostructure is lined with partially-complementary single-stranded nucleic acid, referred to as “anti-handles” (FIG. 1A(iv), bottom, white dotted line representing position of anti-handles). Thus, the interface of the two cylindrical-shaped nanostructures is lined with pH-sensitive single-stranded nucleic acids. In an aqueous environment having a pH of less than 6, a nucleic acid nanocapsule remains in a “closed” configuration (FIG. 1A(ii)) due to dimerization/hybridization of pH-sensitive nucleic acids positioned at an interface between the two nanostructures that form a nanocapsule (FIG. 1A(ii)). When the pH of the aqueous environment decreases to less than 6, the nucleic acid nanocapsule “opens” (e.g., to release the contents of its interior compartment) (FIG. 1A(iii)). Opening of the nanocapsule is due to dissociation of pH-sensitive handles and anti-handles.

A nucleic acid nanocapsule is considered to be “opened” if linkage of one nanostructure of the nanocapsule separates, or partially separates, from another nanostructure of the nanocapsule. Opening of a nanocapsule permits cargo present in the interior compartment of the nanocapsule to be released. With reference to FIG. 1A(iv), as an example, two of the nanostructures that form the nanocapsule have been partially separated from each other. Thus, the nanocapsule is open. In some embodiments, a nanocapsule opens through partially separation of two nanostructures that form the nanocapsule. In some embodiments, a nanocapsule opens through complete separation of two nanostructures that form the nanocapsule.

Handles and anti-handles of the present disclosure (e.g., pH-sensitive handles and anti-handles) may be partially-complementary to each other or wholly-complementary to each other, meaning that each single-stranded nucleic acid contains a region that is partially or wholly complementary to each other such that a handle and an anti-handle dimerize/hybridize at a pH of greater than 6 and dissociate at a pH of less than 6. As used herein, the more general term “complementary” encompasses wholly-complementary and partially-complementary arrangements. Two single-stranded nucleic acids, or regions of two single-stranded nucleic acids, are considered wholly-complementary to each other if there is 100% base-pair complementarity between the two. Conversely, two single-stranded nucleic acids, or regions of two single-stranded nucleic acids, are considered partially-complementary to each other if there is less than 100% base-pair complementarity between the two and the two nucleic acids hybridize to each other under suitable conditions. For example, two single-stranded nucleic acids are considered partially complementary if there is 50% to 99%, 50% to 98%, 50% to 95%, 50% to 90%, 50% to 85%, 50% to 80%, 50% to 75%, 50% to 70%, 50% to 65%, or 50% to 60% base-pair complementarity between the two nucleic acids, or between two regions of the nucleic acids, and the two nucleic acids hybridize to each other under suitable conditions. In some embodiments, suitable conditions include an aqueous solution having a pH of greater than 6 (e.g., a neutral pH of 7). In some embodiments, suitable conditions include an aqueous solution having a pH of greater than 6 (e.g., a neutral pH of 7) and a temperature of about 25° C. to 37° C.

In some embodiments, pH-sensitive handles comprise, or consist of, the following sequence: 5′-CCCTAACCCTAACCCTAACCC-3′ (SEQ ID NO: 1), referred to as an “i-motif.” I-motif DNA transforms from a B-DNA double helix arrangement into a single-stranded arrangement upon a decrease in pH to less than 6. At a pH of greater than 6, i-motif handles and anti-handles dimerize, and at a pH of less than 6 (e.g., pH of 5.5.), i-motif handles and anti-handles dissociate.

In some embodiments, anti-handles are partially-complementary to pH-sensitive handles comprising, or consisting of, the sequence of SEQ ID NO: 1. In some embodiments, anti-handles are partially-complementary to pH-sensitive handles comprising the sequence of SEQ ID NO: 1 and have one or more nucleotide mismatches. For example, in some embodiments, an anti-handle comprises the following sequence: 5′-TTGTTAGTGTTAGTGTTAGGG-3′ (SEQ ID NO: 2), which is partially-complementary to a pH-sensitive handle comprising the sequence of SEQ ID NO: 1 and has 4 nucleotide mismatches. In some embodiments, an anti-handle comprises the following sequence: 5′-GGTTTATTGTTAGGTTTAGTT-3′ (SEQ ID NO: 3), which is partially-complementary to a pH-sensitive handle comprising the sequence of SEQ ID NO: 1 and has 6 nucleotide mismatches.

The number of nucleotide mismatches between a pH-sensitive handle and an anti-handle can be used to fine-tune the kinetics of opening a nanocapsule. Thus, the number of mismatches between a pH-sensitive handle and an anti-handle may vary. In some embodiments, the number of nucleotide mismatches between a pH-sensitive handle and an anti-handle is 1, 2, 3, 4, 5, 6, 7, 8, 9 or 10. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30% of the nucleotide base pairs between a pH-sensitive handle and a partially-complementary anti-handle are mismatches. A “mismatch” refers to a pair of nucleotides that are not complementary to each other. That is, they do not normally hybridize to each other. Examples of nucleotide mismatches include A-C, A-G, T-C and T-G.

Handles and/or anti-handles may be 15 to 50 nucleotides in length. In some embodiments, a handle and/or anti-handle is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 60 nucleotides in length. Depending on the application, a handle and/or anti-handle may be greater than 50 nucleotides in length. Thus, the length of a handle and/or anti-handle may vary.

The extent to which and rate at which handles and/or anti-handles dissociate at a particular pH can be rationally designed. Factors to consider include length of the single-stranded nucleic acids and number of nucleotide mismatches. In some embodiments, handles and anti-handles are designed to dissociate at a pH of 5.9, 5.8, 5.7, 5.6, 5.5, 5.4, 5.3, 5.2, 5.1 or 5.0.

In some embodiments, handles and anti-handles are designed to dissociate, resulting in opening of a nanocapsule within 20 seconds (s) to 5 minutes (min) of entry into a cell (e.g., uptake by a cell). In some embodiments, a nanocapsule opens within 20 s, 30 s, 40 s, 50 s, 60 s, 70 s, 80 s, 90 s 100 s, 110 s, 2 min, 3 min, 4 min or 5 min.

Handles of the present disclosure may be linked to nucleic acid nanocapsules through covalent or non-covalent linkages. Similarly, anti-handles of the present disclosure may be linked to agents through covalent or non-covalent linkages.

Examples of non-covalent linkages include, without limitation, binding between protein binding partners, such as, for example, biotin and avidin/streptavidin and nucleic acid hybridization interactions, also referred to as Watson-Crick nucleotide base pairing interactions. In some embodiments, a handle may be linked non-covalently to the nanocapsule using biotin and avidin/streptavidin. Other binding pairs will be apparent to those of ordinary skill in the art and may be used for linking a handle to a nanocapsule and/or an anti-handle to an agent, including high affinity protein/protein binding pairs such as antibody/antigen and ligand/receptor binding pairs. Handles may be designed to partially hybridize to a nucleic acid nanocapsule. For example, a portion of the handle may be complementary to a region of the nanostructure such that it hybridizes to the nanostructure, and another portion of the handle may be partially complementary to an anti-handle.

Examples of covalent linkages include, without limitation, thiol-maleimide crosslinker chemistry (e.g., covalent linkage between thiol, e.g., cysteine, on an agent and maleimide on an anti-handle (e.g., generated by reacting amino-DNA with succinimidyl-4-(N-maleimidomethyl)cyclohexane-1-carboxylate (SMCC), which is an amine-to-sulfhydryl crosslinker that contains NHS-ester and maleimide reactive groups at opposite ends of a medium-length cyclohexane-stabilized spacer arm), covalent linkage catalyzed in trans by enzyme, linking protein to DNA (e.g., ybbR tag on protein is linked to CoA-modified nucleic acid) (see, e.g., Yin, J. et al. Nat. Protoc. 1(1): 280-5, 2006), and enzymatic addition of modified NTPs to nucleic acid (see, e.g., Sorensen, R. S. et al. ACS Nano 7(9): 8098-104, 2013).

Handles and anti-handles are positioned at the interface of two nucleic acid nanostructures that form a nanocapsule. In some embodiments, handles and anti-handles are regularly spaced along the inside surface and/or exterior surface of an interface between two nucleic acid nanostructures. For example, for a 60 nm diameter nanostructure (where total circumference is pi*diameter=3.14*60 nm=190 nm), 12 pairs of handles/anti-handles, may be regularly spaced on the inside or outside of an interface. In some embodiments, for a 60 nm diameter nanostructure, 24 pairs of handles/anti-handles, may be regularly spaced on the inside or outside of an interface. In some embodiments, the handles/anti-handles are not regularly spaced, and therefore, are irregularly spaced. It should be understood that the present disclosure contemplates various handle/anti-handle configurations that permit opening of a nanocapsule upon entry into a cell (e.g., within 20 seconds to 5 minutes).

Release of agent by a nucleic acid nanocapsule from its interior compartment (and/or from its exterior surface) is dependent on changes in pH. For example, as shown in FIG. 5A, an agent is linked to a nanocapsule indirectly through pH-sensitive handle/anti-handle interactions. The nanocapsule depicted in FIG. 5A is decorated with pH-sensitive handles, and an agent is conjugated (e.g., covalently conjugated) to partially-complementary anti-handles. At a neutral pH (such as that of physiological conditions), for example, handles and anti-handles dimerize, thereby linking the agent to the nanocapsule. As the pH drops to below 6 (e.g., as the nanocapsule enters an endosomal compartment of a cell), the handle folds up on itself and dissociates from the anti-handle, thereby “releasing” the anti-handle-agent conjugate, with the end result that the handles and anti-handles are no longer hybridized to each other and the two (or more) nanostructures are no longer linked to each other to form a closed capsule.

### Uses

Nucleic acid nanostructures (e.g., nanocapsules) of the present disclosure have a variety of in vitro and in vivo uses. In some embodiments, may be used as scaffolds, cages or multifunctional carriers for delivering an agent (e.g., therapeutic agent) that is intended for use in vivo and/or in vitro. A nucleic acid nanostructure may be delivered by any suitable delivery method, for example, intravenously or orally. As used herein, an agent is any atom, molecule, or compound that can be used to provide benefit to a subject (including without limitation prophylactic or therapeutic benefit) or that can be used for diagnosis and/or detection (for example, imaging) in vivo, or that may be used for effect in an in vitro setting (for example, a tissue or organ culture, a clean-up process, and the like). Agents may be, without limitation, therapeutic agents and/or diagnostic agents. In some embodiments, an agent is a therapeutic agent, a prophylactic agent and/or a diagnostic agent. In some embodiments, an agent is a targeting molecule. Examples of agents for use with any one of the embodiments described herein are described below.

The present disclosure contemplates imparting addressability to nucleic acid nanostructures. For example, nucleic acid nanostructures may be modified by site-specific attachment of targeting moieties such as proteins, ligands or other small biomolecules. In some embodiments, nucleic acid nanostructures may comprise nucleic acid “staple” strands, as described above, that serve as handles for nanometer-specific placement of accessory molecules (e.g., biotin/streptavidin) at virtually any position on or within the structure (see, e.g., Stein et al. Chemphyschem. 12(3), 689-695 (2011); Steinhauer et al. Angew Chem. Int. Ed. Engl. 48(47), 8870-8873 (2009); Stein et al. J. Am. Chem. Soc. 133(12), 4193-4195 (2011); Kuzyk et al. Nature 483(7389), 311-314 (2012); and Ding et al. J. Am. Chem. Soc. 132(10), 3248-3249 (2010); Yan et al. Science 301(5641), 1882-1884 (2003); and Kuzuya et al. Chembiochem. 10(11), 1811-1815 (2009), each of which is incorporated by reference herein).

In some embodiments, nucleic acids of nanostructures provided herein may be modified (e.g., covalently modified) with a linker (e.g., biotin linker) during synthesis or via enzymatic means (see, e.g., Jahn et al. Bioconjug. Chem. 22(4), 819-823 (2011) incorporated by reference herein). Such methods may also be used to position reaction systems on nucleic acid nanostructures through the chemical biotinylation of enzyme molecules (see, e.g., Voigt et al. Nat. Nanotechnol. 5(3), 200-203 (2010)).

A more generalized antibody-based binding approach may also be used to link target proteins to nucleic acid nanostructures at defined distances (see, e.g., Williams et al. Angew Chem. Int. Ed. Engl. 46(17), 3051-3054 (2007); and He Y et al. J. Am. Chem. Soc. 128(39), 12664-12665 (2006), each of which is incorporated by reference herein). Thus, in some embodiments, nucleic acid nanostructures may be linked to one or more antibodies.

In other embodiments, DNA aptamers, which adopt a specific secondary structure with high binding affinity for a particular molecular target, may be used as linkers, thereby eliminating the need for protein linkers (see, e.g., Ellington et al. Nature 346(6287), 818-822 (1990); Chhabra et al. J. Am. Chem. Soc. 129(34), 10304-10305 (2007); and Rinker et al. Nat. Nanotechnol. 3(7), 418-422 (2008), each of which is incorporated by reference herein).

The present disclosure also contemplates the use of recombinant genetic engineering methods to selectively add affinity tags or other peptide linkers to nucleic acid nanostructures. For example, polyhistidine sequence consisting of multiple histidine residues on the C- or N-terminus end of a target protein is a commonly used tag for affinity-based purification. This, in turn, can be linked via nickel-mediated interaction to a nitrilotriacetic acid molecule that is covalently conjugated to an amine—(see, e.g., Goodman et al. Chembiochem. 10(9), 1551-1557 (2009), incorporated by reference herein) or thiol-modified (see, e.g., Shen et al. J. Am. Chem. Soc. 131(19), 6660-6661 (2009), incorporated by reference herein) nucleic acid. Through this method, fluorescent proteins may be positioned both periodically and specifically on nucleic acid nanostructures (Goodman et al. (2009); and Shen et al. (2009)). Similarly, SNAP and HaloTag® peptide sequences, also used for affinity purification of recombinant proteins, may be utilized for the orthogonal decoration of nucleic acid nanostructures with different protein or enzyme species (see, e.g., Sacca et al. Angew Chem. Int. Ed. Engl. 49(49), 9378-9383 (2010), incorporated by reference herein). A related approach involving the creation of chimeric proteins conjugated to a DNA-binding domain, can eliminate the often complex chemical synthesis techniques and toxic compounds (e.g., nickel) necessary to stably conjugate affinity tag binding partners to oligonucleotide strands. Further, zinc-finger domains that recognize specific double-stranded sequences may be used to arrange fluorescent proteins at specific locations on nucleic acid nanostructures of the present disclosure (see, e.g., Nakata et al. Angew Chem. Int. Ed. Engl. 51(10), 2421-2424 (2012), incorporated by reference herein).

An agent may be covalently or non-covalently attached to a nucleic acid nanostructure. The location and nature of the linkage between the agent and the nucleic acid nanostructure will depend upon the function of the agent. As an example, an agent may be intended to release (including slow release) from the nanostructure, and in that case, the linkage between the agent and the nanostructure may be chosen to achieve the desired release profile.

In some embodiments, an agent may be inactive in its bound form and activated only when released.

In some embodiments, an agent may be combined with nucleic acids during assembly (e.g., self-assembly) of nanostructures, or an agent may be combined with pre-formed nucleic acid nanostructures.

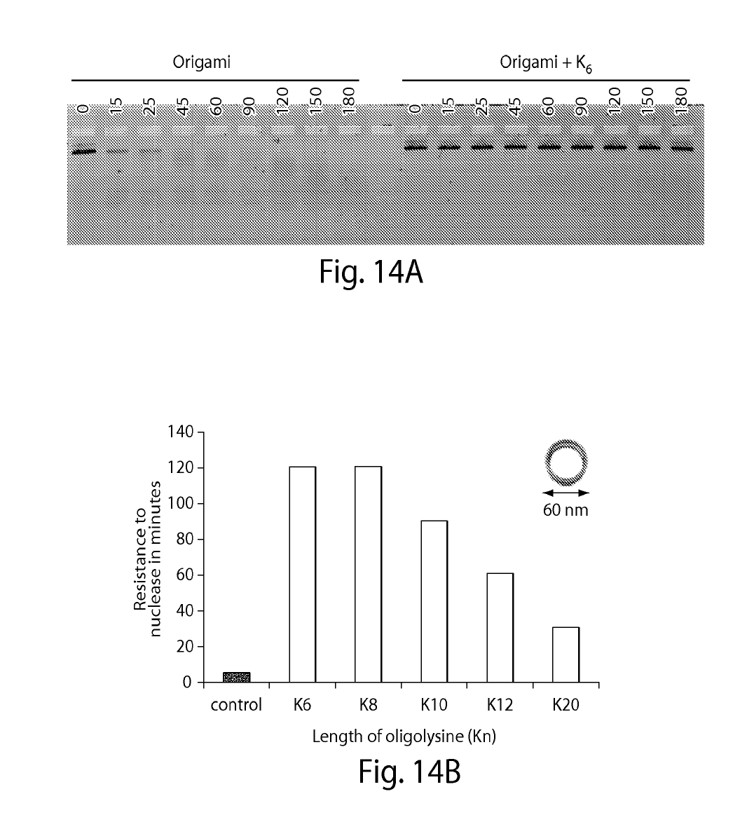

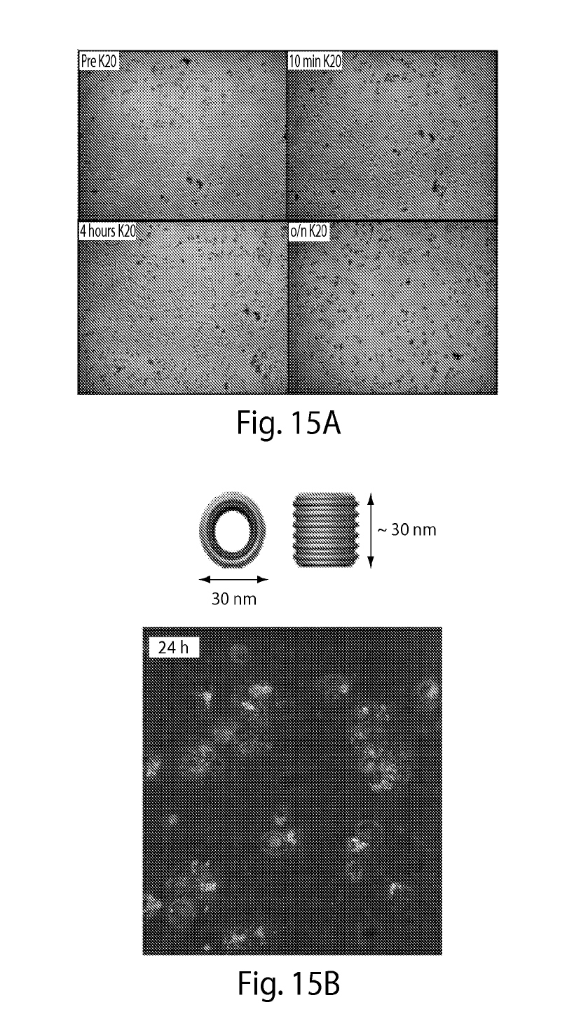

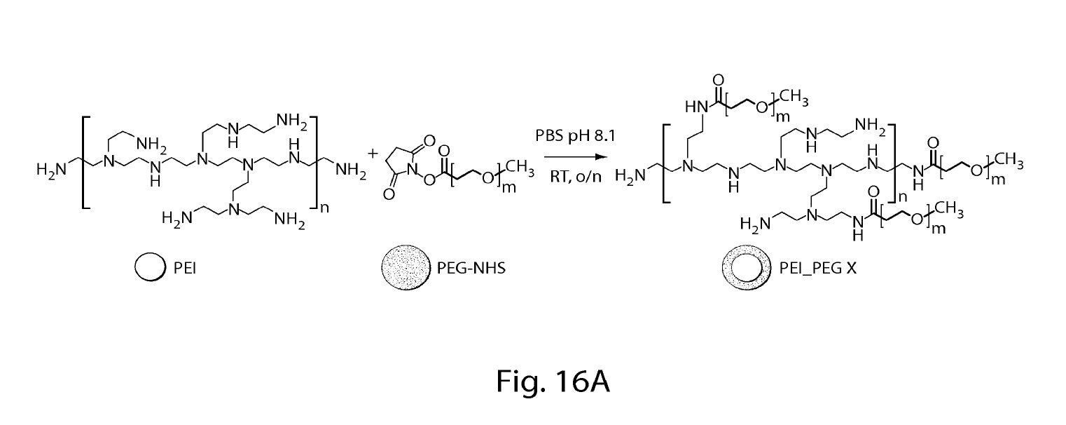

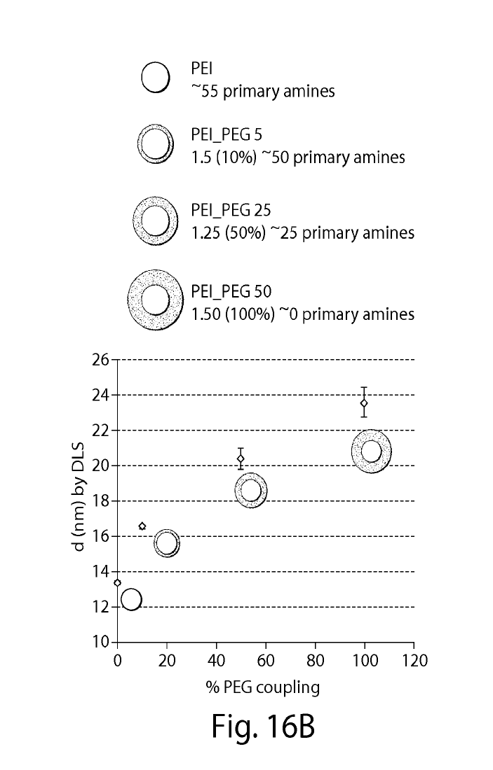

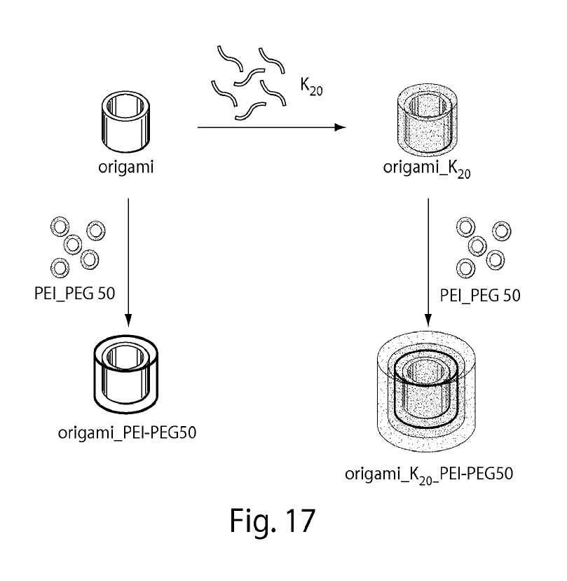

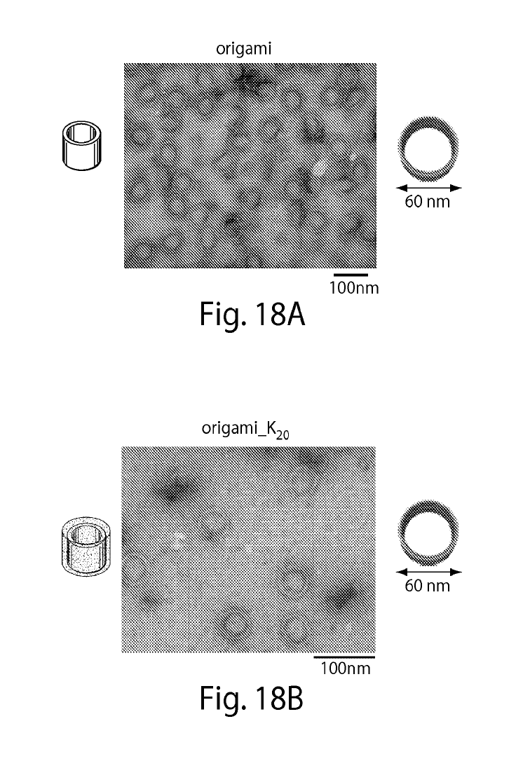

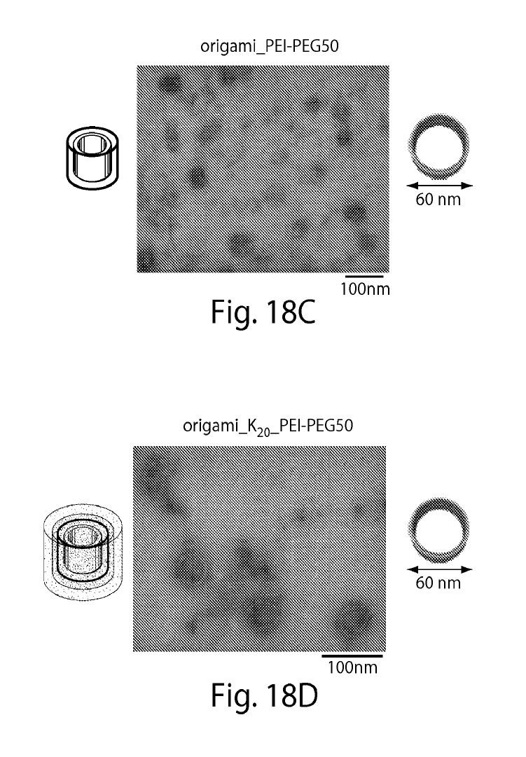

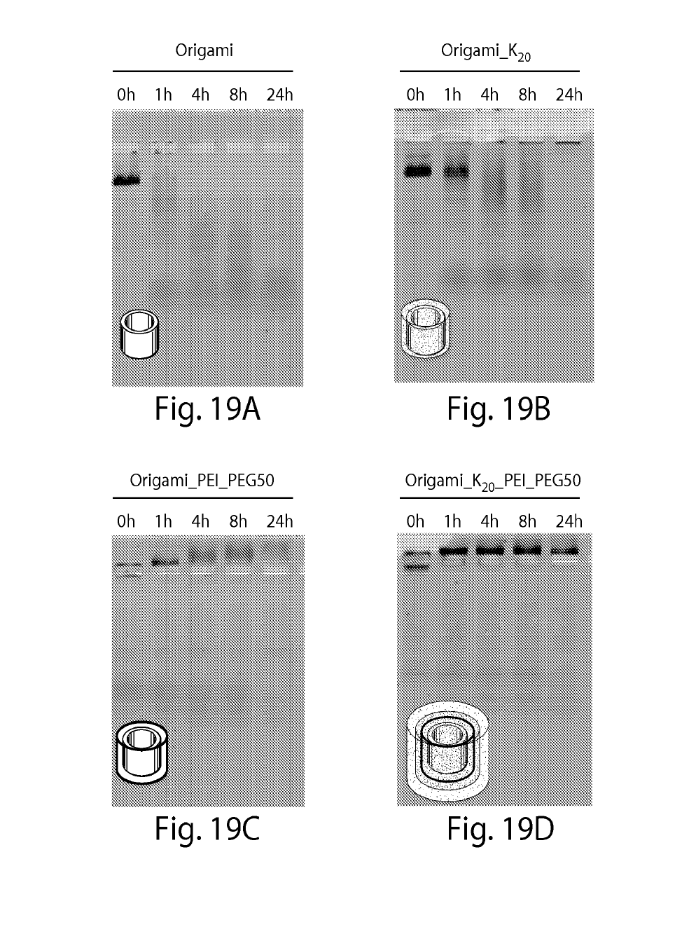

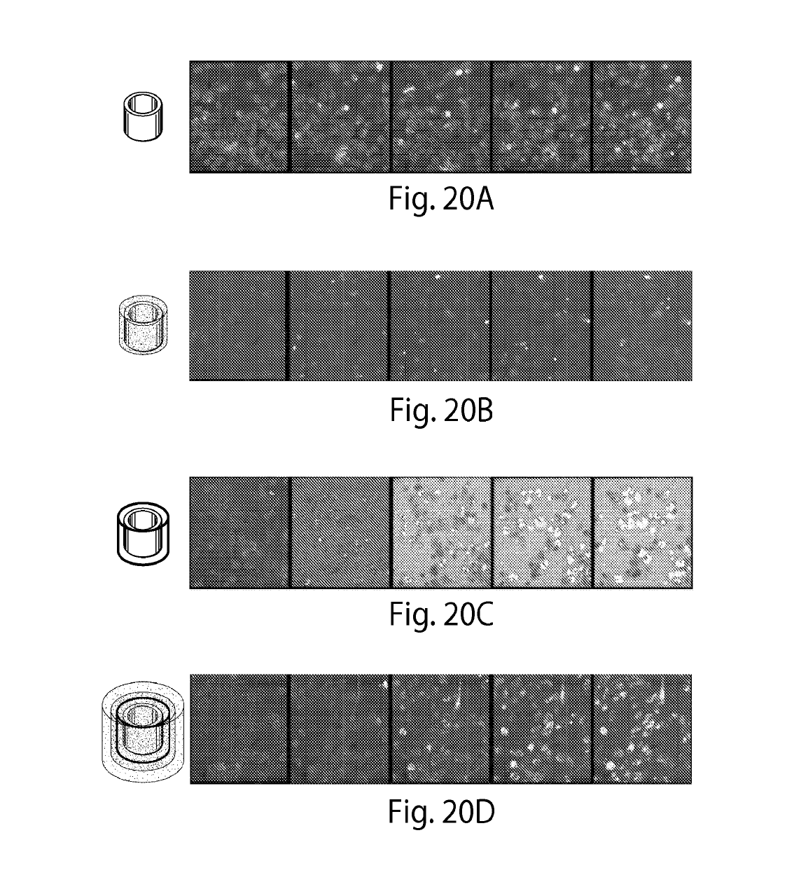

Agents may be linked to an interior surface (in the interior compartment) or an exterior surface of a nanostructure (e.g., nanocapsule). Agents may be arranged in various configurations. FIG. 14A depicts the interior surface and the exterior interface of a cross section of a segment of a nucleic acid nanocapsule of the present disclosure having handles linked to both surfaces (left). Also shown in FIG. 14A are examples of agents (e.g., antibody, “danger” signals, imaging agents, antigen) linked to anti-handles. Upon hybridization of handles to anti-handles, agents become indirectly linked to nucleic acid nanocapsules. In some embodiments, as shown in FIG. 14B, the exterior surface of a nanocapsule (left) contains a combination of adjuvant molecules (e.g., CpG oligonucleotides) and targeting molecules (e.g., antibody fragments such as scFV fragments), and the interior surface of the nanocapsule (right) contains a combination of tracking dye and antigen. It should be understood that nanostructures (e.g., nanocapsules) of the present disclosure permit precise placement of an agent or more than one agent (e.g., a combination of different agents) on the interior and/or exterior surface of the nanostructures (e.g., nanocapsules) (see, e.g., FIG. 14C).

Nucleic acid nanostructures (e.g., nanocapsules) of the present disclosure permit high-density “packing” of agent on and into the nanocapsules. In some embodiments, a nucleic acid nanostructures (e.g., nanocapsules) is decorated with one agent per 50 nm² to 75 nm². In some embodiments, a nucleic acid nanocapsule is decorated with one agent per 50 nm², 55 nm², 60 nm², 65 nm², 70 nm² or 75 nm². For example, using a rhombic-lattice spacing, as shown in FIG. 14B, for a 30 nm tall, 60 nm diameter cylindrical nanostructure, 72 positions on the exterior of the nanostructure and 84 positions on the interior may be occupied by agent. For larger nanostructures (e.g., nanocapsules), for example, those with two 30 nm×60 nm cylindrical nanostructures, the number of positions occupied by agent is doubled. For even larger nanostructures (e.g., nanocapsules), for example, those with three 30 nm×60 nm cylindrical nanostructures, the number of positions occupied by agent tripled, and so on.

The present disclosure contemplates, in some aspects, the delivery of nucleic acid nanostructures, or nucleic acid nanostructures loaded with an agent, systemically or to localized regions, tissues or cells. Any agent may be delivered using the methods of the present disclosure provided that it can be loaded onto or into the nucleic acid nanostructure. Because such processes are relatively innocuous, it is expected that virtually any agent may be used.

An agent for use in accordance with the present disclosure may be a protein-based agent (including a protein), a nucleic-acid based agent (including a nucleic acid), a chemical-based agent (including chemical compounds) or combination of any two or more of the foregoing. For example, an agent may be an antibody-drug conjugate. An “antibody-drug conjugate” is a complex of an antibody (e.g., a whole monoclonal antibody (mAb) or an antibody fragment such as a single-chain variable fragment (scFv)) linked to a biologically-active small molecule (e.g., small molecule drug).

Examples of protein-based and peptide-based agents (e.g., therapeutic, prophylactic and/or diagnostic protein-based agents) for use in accordance with the present disclosure include, without limitation, antibodies (e.g., monoclonal antibodies, chimeric antibodies, humanized antibodies), antibody fragments (e.g., single- or multi-chain antibodies, antibody fragments such as Fab fragments, Fc fragments), enzymes, co-factors, receptors, ligands, transcription factors and other regulatory factors, antigens, cytokines, chemokines and hormones.

Examples of nucleic acid-based agents (e.g., therapeutic, prophylactic and/or diagnostic nucleic acid-based agents) for use in accordance with the present disclosure include, without limitation, RNA interference molecules such as short-interfering RNA (siRNA) molecules, short-hairpin RNA (shRNA) molecules, and micro RNA (miRNA) molecules. Nucleic acid-based agents may be recombinant (e.g., non-naturally occurring molecule produced by joining two different nucleic acids) or synthetic (e.g., chemically or otherwise synthesized).

Examples of chemical-based agents (e.g., therapeutic, prophylactic and/or diagnostic chemical-based agents) for use in accordance with the present disclosure include, without limitation, small molecules (e.g., small molecule drugs). A “small molecule” is a low molecular weight (e.g., <900 Daltons) organic compound.

A “therapeutic agent” is an agent used to treat a condition in a subject (e.g., human or non-human subject). A “prophylactic agent” is an agent used to prevent a condition in a subject (e.g., human or non-human subject). Examples of therapeutic agents and prophylactic agents for use in accordance with the present disclosure include, without limitation, antibodies, antibody fragments, other proteins and peptides, lipids, carbohydrates, small molecules, polymers, metal nanoparticles, RNA interference molecules (e.g., siRNAs, shRNAs, miRNAs), antisense molecules, antigens (e.g., peptide antigens), adjuvants (e.g., CpG oligonucleotides), anti-neoplastic agents, anti-cancer, anti-infective agents (e.g., anti-microbial agents, anti-bacterial agents), anti-fungal agents, anti-viral agents (e.g., anti-retroviral agents), anti-inflammatory agents, metabolic agents, immunomodulatory agents (e.g., immunostimulatory agents, immunosuppressive agents), anti-hypertensive agents, anti-Alzheimer's agents, and anti-Parkinson's agents.

An “adjuvant” is an agent that enhances an immune response to an antigen. In some embodiments, an adjuvant is a CpG oligonucleotide. CpG oligonucleotides are short single-stranded synthetic DNA molecules that contain a cytosine triphosphate deoxynucleotide (“C”) followed by a guanine triphosphate deoxynucleotide (“G”). The “p” refers to the phosphodiester, or modified phosphorothioate (PS), linkage between consecutive nucleotides. CpG oligonucleotides typically enhance the immunostimulatory effect of nucleic acid nanostructures (Li, J. et al. ACS NANO, 5(11): 8783-8789, 2011; Schuller, V. et al. ACS NANO, 5(12): 9696-9702, 2011). For example, after they are taken up by cells, CpG oligonucleotides, which are a hallmark of microbial DNA, are recognized by the endosomal Toll-like receptor 9 (TLR9) that activates downstream pathways to induce immunostimulatory effects, producing high-level secretion of various pro-inflammatory cytokines including tumor necrosis factor (TNF)-α, interleukin (IL)-6, and IL-12. In some embodiments, CpG oligonucleotides are linked to an interior surface of a nucleic acid nanocapsule. In some embodiments, CpG oligonucleotides are linked to an exterior surface of a nucleic acid nanocapsule. In some embodiments, a nucleic acid nanocapsule has CpG oligonucleotides linked to both an interior and exterior surface. Other examples of adjuvants include, without limitation, lipopolysaccharide and polyI:C (dsRNA mimic).

A “diagnostic agent” is an agent used to diagnose a condition in a subject (e.g., human or non-human subject). Examples of therapeutic agents and prophylactic agents for use in accordance with the present disclosure include, without limitation, imaging agents (e.g., contrast agents, radioactive agents, tracking dyes (e.g., fluorescent dyes)). An “imaging agent” is an agent that emits signal directly or indirectly thereby allowing its detection in vivo. Imaging agents, such as contrast agents and radioactive agents, can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include 201 Tl, gamma-emitting radionuclide 99 mTc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203Pb, and 11 ln; imaging agents for in vivo fluorescence imaging such as fluorescent dyes or dye-conjugated nanoparticles. In some embodiments, an agent to be delivered is conjugated, or fused to, or mixed or combined with an imaging agent.

An agent may be naturally occurring or non-naturally occurring (e.g., chemical compounds that are non-naturally occurring). Naturally occurring agents include those capable of being synthesized by the subjects to whom nucleic acid nanostructures are administered. Non-naturally occurring are those that do not exist in nature normally, whether produced by plant, animal, microbe or other living organism. It should be understood that nucleic acid nanocapsules that comprise naturally-occurring agents are considered, as a whole, to be non-naturally occurring.

An agent may be, without limitation, a chemical compound including a small molecule, a protein, a polypeptide, a peptide, a nucleic acid (e.g., siRNA, shRNA, microRNA), a virus-like particle, a steroid, a proteoglycan, a lipid, a carbohydrate, and analogs, derivatives, mixtures, fusions, combinations or conjugates thereof. An agent may be a prodrug that is metabolized and thus converted in vivo to its active (and/or stable) form. The present disclosure further contemplates the loading of more than one type of agent in a nucleic acid nanostructure and/or the combined use of nanostructures comprising different agents.

A variety of agents that are currently used for therapeutic or diagnostic purposes can be delivered according to the present disclosure and these include, without limitation, imaging agents, immunomodulatory agents such as immunostimulatory agents and immunoinhibitory agents (e.g., cyclosporine), antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, analgesics, opioids, enzyme inhibitors, neurotoxins, hypnotics, anti-histamines, lubricants, tranquilizers, anti-convulsants, muscle relaxants, anti-Parkinson agents, anti-spasmodics, muscle contractants including channel blockers, miotics and anti-cholinergics, anti-glaucoma compounds, modulators of cell-extracellular matrix interactions including cell growth inhibitors and anti-adhesion molecules, vasodilating agents, inhibitors of DNA, RNA or protein synthesis, anti-hypertensives, antipyretics, steroidal and non-steroidal anti-inflammatory agents, anti-angiogenic factors, antisecretory factors, anticoagulants and/or antithrombotic agents, local anesthetics, ophthalmics, prostaglandins, targeting agents, neurotransmitters, proteins, cell response modifiers and vaccines. It should be understood that any one or more of the foregoing agents may be specifically excluded from nucleic acid nanostructures of the present disclosure.

A “targeting molecule” is a molecule that directs a nucleic acid nanostructure (e.g., nanocapsule) to a target cell of interest. Targeting molecules that target cell types, such as, for example, dendritic cells, tumor cells, T cells, B cells and natural killer (NK) cells are contemplated herein. In some embodiments, a targeting molecule binds specifically to extracellular cognate molecules present on on target cells. Examples of targeting molecule for use in accordance with the present disclosure include, without limitation, antibodies, antibody fragments and ligands.

In some embodiments, a targeting molecule directs a nucleic acid nanostructure (e.g., nanocapsule) specifically to dendritic cells (DCs). In some embodiments, a targeting molecule binds specifically to DEC205, which is enriched on target dendritic cells. In some embodiments, a targeting molecule is a single chain antibody fragments (scFv) that binds specifically to DEC205. In some embodiments, a targeting molecule binds specifically to a subset of DCs such as, for example, BDCA3⁺ cells, Langerhans cells, Dermal CD1a⁺ cells, BDCA1⁺ cells, moDC cells or pDC cells. In some embodiments, a targeting molecule binds specifically to Clec9A, Clec12A, DCAR1, Dectin1, Dectin2, DCIR, DC-SIGN, Langerin, MGL, MR, Siglec-H, BST-2 or BDCA-2 (Kreutz, M. et al. Blood, 121(15): 2836, 2013).

In some embodiments, a targeting molecule directs a nucleic acid nanostructure (e.g., nanocapsule) specifically to tumor cells. In some embodiments, a targeting molecule binds specifically to an alpha(v)beta(3) integrin, a folate receptor and/or an epidermal growth factor receptor, for example, present on the surface of tumor cells. In some embodiments, a targeting molecule is a RGD (Arg-Gly-Asp) peptide (e.g., cRGD peptide).

In some embodiments, a targeting molecule directs a nucleic acid nanostructure (e.g., nanocapsule) specifically to stem cells. In some embodiments, a targeting molecule binds specifically to CD34 or CD117, for example, present on the surface of stem cells (e.g., CD34⁺, CD31⁻, CD117⁺ stem cells).

In some embodiments, a targeting molecule directs a nucleic acid nanostructure (e.g., nanocapsule) specifically to leukocytes. In some embodiments, a targeting molecule binds specifically to CD45⁺, for example, present on the surface of leukocytes (e.g., granulocytes, monocytes, T lymphocytes, T regulatory cells, cytotoxic T cells, B lymphocytes, thrombocytes, and/or natural killer (NK) cells).

In some embodiments, a targeting molecule directs a nucleic acid nanostructure (e.g., nanocapsule) specifically to granulocytes. In some embodiments, a targeting molecule binds specifically to CD45, CD11b, CD15, CD24, CD114 or CD182, for example, present on the surface of granulocytes (e.g., CD45⁺, CD11b⁺, CD15⁺, CD24⁺, CD114+⁺, CD182⁺ granulocytes).

In some embodiments, a targeting molecule directs a nucleic acid nanostructure (e.g., nanocapsule) specifically to monocytes. In some embodiments, a targeting molecule binds specifically to CD45, CD14, CD114, CD11a, CD11b, CD91 or CD16, for example, present on the surface of monocytes (e.g., CD45⁺, CD14⁺, CD114⁺, CD11a⁺, CD11b⁺, CD91⁺ or CD16⁺ monocytes).

In some embodiments, a targeting molecule directs a nucleic acid nanostructure (e.g., nanocapsule) specifically to T lymphocytes. In some embodiments, a targeting molecule binds specifically to CD45 or CD3, for example, present on the surface of T lymphocytes (e.g., CD45⁺, CD3⁺ T lymphocytes).

In some embodiments, a targeting molecule directs a nucleic acid nanostructure (e.g., nanocapsule) specifically to T helper cells. In some embodiments, a targeting molecule binds specifically to CD45⁺, CD3⁺ or CD4⁺, for example, present on the surface of T helper cells (e.g., CD45⁺, CD3⁺, CD4⁺ T helper cells).

In some embodiments, a targeting molecule directs a nucleic acid nanostructure (e.g., nanocapsule) specifically to T regulatory cells. In some embodiments, a targeting molecule binds specifically to CD4, CD25 or Foxp3, for example, present on the surface of T regulatory cells (e.g., CD4⁺, CD25⁺, Foxp3⁺ T regulatory cells).

In some embodiments, a targeting molecule directs a nucleic acid nanostructure (e.g., nanocapsule) specifically to cytotoxic T cells. In some embodiments, a targeting molecule binds specifically to CD45, CD3 or CD8, for example, present on the surface of cytotoxic T cells (e.g., CD45⁺, CD3⁺, CD8⁺ cytotoxic T cells).

In some embodiments, a targeting molecule directs a nucleic acid nanostructure (e.g., nanocapsule) specifically to B lymphocytes. In some embodiments, a targeting molecule binds specifically to CD45, CD19, CD45, CD20, CD24, CD38 or CD22, for example, present on the surface of B lymphocytes (e.g., CD45⁺, CD19⁺ or CD45⁺, CD20⁺, CD24⁺, CD38⁺, CD22⁺ B lymphocytes).

In some embodiments, a targeting molecule directs a nucleic acid nanostructure (e.g., nanocapsule) specifically to thrombocytes. In some embodiments, a targeting molecule binds specifically to CD45 or CD61, for example, present on the surface of thrombocytes (e.g., CD45⁺, CD61⁺ thrombocytes).

In some embodiments, a targeting molecule directs a nucleic acid nanostructure (e.g., nanocapsule) specifically to NK cells. In some embodiments, a targeting molecule binds specifically to CD16, CD56, CD31, CD30 or CD38, for example, present on the surface of NK cells (e.g., CD16⁺, CD56⁺, CD3⁻, CD31⁺, CD30⁺, CD38⁺ NK cells).

In some embodiments, a targeting molecule directs a nucleic acid nanostructure (e.g., nanocapsule) specifically to neural stem cells. In some embodiments, a targeting molecule binds specifically to ABCG2, NeuroD1, ASCL1/Mash1, Noggin, Beta-catenin, Notch-1, Notch-2, Brg1, Nrf2, N-Cadherin, Nucleostemin, Calcitonin R, Numb, CD15/Lewis X, Otx2, CDCP1, Pax3, COUP-TF I/NR2F1, Pax6, CXCR4, PDGF R alpha, FABP7/B-FABP, PKC zeta, FABP 8/M-FABP, Prominin-2, FGFR2, ROR2, FGFR4, RUNX1/CBFA2, FoxD3, RXR alpha/NR2B1, Frizzled-9, sFRP-2, GATA-2, SLAIN 1, GCNF/NR6A1, SOX1, GFAP, SOX2, Glut1, SOX9, HOXB1, SOX11, ID2, SOX21, Meteorin, SSEA-1, MSX1, TRAF-4, Musashi-1, Vimentin, Musashi-2, ZIC1 or Nestin, for example, present on the surface of neural stem cells.

In some embodiments, a targeting molecule directs a nucleic acid nanostructure (e.g., nanocapsule) specifically to neural progenitor cells. In some embodiments, a targeting molecule binds specifically to A2B5, AP-2 Alpha, ATPase Na+/K+ transporting alpha 1, Activin RIIA, Brg1, CD168/RHAMM, CD4, Doublecortin/DCX, Frizzled 4/CD344, GAP43, Jagged1, Laminin, MSX1/HOX7, Mash1, Musashi-1, Nestin, Netrin-1, Netrin-4, Neuritin, NeuroD1, Neurofilament alpha-internexin/NF66, Notch1, Notch2, Notch3, Nucleostemin, Otx2, PAX3, S100B, SOX2, Semaphorin 3C, Semaphorin 6A, Semaphorin 6B, Semaphorin 7A, TROY/TNFRSF19, Tubulin βII, Tuj 1 or Vimentin, for example, present on the surface of, neural progenitor cells.

In some embodiments, a targeting molecule directs a nucleic acid nanostructure (e.g., nanocapsule) specifically to early neuronal cells. In some embodiments, a targeting molecule binds specifically to ATH1/MATH1, ASH1/MASH1, HES5, HuC/Hu, HuD, Internexin α, L1 neural adhesion molecule, MAP1B/MAPS, MAP2A, MAP2B, Nerve Growth Factor Rec/NGFR), Nestin, NeuroD, Neurofilament L 68 kDa, Neuron Specific Enolase/NSE, NeuN, Nkx-2.2/NK-2, Noggin, Pax-6, PSA-NCAM, Tbr1, Tbr2, Tubulin βIII, TUC-4, or Tyrosine hydroxylase/TH, for example, present on the surface of early neuronal cells.

In some embodiments, a targeting molecule directs a nucleic acid nanostructure (e.g., nanocapsule) specifically to immature neurons and/or growth cones. In some embodiments, a targeting molecule binds specifically to Collapsin Response Mediated Protein 1/CRMP1, Collapsin Response Mediated Protein 2/CRMP2, Collapsin Response Mediated Protein 5/CRMP5, Contactin-1, Cysteine-rich motor neuron 1/CRIM1, c-Ret phosphor Serine 696, Doublecortin/DCX, Ephrin A2, Ephrin A4, Ephrin A5, Ephrin B1, Ephrin B2, GAP-43, HuC, HuD, Internexin alpha, Laminin-1, LINGO-1, MAP1B/MAPS, Mical-3, NAP-22, NGFR, Nestin, Netrin-1, Neuropilin, Plexin-A1, RanBPM, Semaphorin 3A, Semaphorin 3F, Semaphorin 4D, Slit2, Slit3, Staufen, Tbr 1, Tbr 2, Trk A, Tubulin βIII or TUC-4, for example, present on the surface of immature neurons and/or growth cones.

In some embodiments, a targeting molecule directs a nucleic acid nanostructure (e.g., nanocapsule) specifically to differentiated post-mitotic neuronal cells. In some embodiments, a targeting molecule binds specifically to NeuN, NF-L, NF-M, GAD, TH, PSD-95, Synaptophysin, VAMP or ZENON, for example, present on the surface of differentiated post-mitotic neuronal cells.

In some embodiments, a targeting molecule directs a nucleic acid nanostructure (e.g., nanocapsule) specifically to motorneurons. In some embodiments, a targeting molecule binds specifically to ChAT/choline acetyltransferase Chox10, En1, Even-skipped/Eve, Evx1, Evx2, Fibroblast growth factor-1/FGF1, HB9, Is11, Is12, Lim3, Nkx6, p75 neurotrophin receptor, REG2, Sim1, SMI32 or Zfh1, for example, present on the surface of motorneurons.

In some embodiments, a targeting molecule directs a nucleic acid nanostructure (e.g., nanocapsule) specifically to perisynaptic regions of neurons. In some embodiments, a targeting molecule binds specifically to 4.1G, Acetylcholinesterase, Ack1, AMPA Receptor Binding Protein/ABP, ARG3.1, Arp2, E-Cadherin, N-Cadherin, Calcyon, Catenin alpha and beta, Caveolin, CHAPSYN-110/PSD93, Chromogranin A, Clathrin light chain, Cofilin, Complexin 1/CPLX1/Synaphin 2, Contactin-1, CRIPT, Cysteine String Protein/CSP, Dynamin 1, Dymanin 2, Flotillin-1, Fodrin, GRASP, GRIP1, Homer, Mint-1, Munc-18, NSF, PICK1, PSD-95, RAB4, Rabphillin 3A, SAD A, SAD B, SAP-102, SHANK1a, SNAP-25, Snapin, Spinophilin/Neurabin-1, Stargazin, Striatin, SYG-1, Synaptic Vesicle Protein 2A, Synaptic Vesicle Protein 2B, Synapsin 1, Synaptobrevin/VAMP, Synaptojanin 1, Synaptophysin, Synaptotagmin, synGAP, Synphilin-1, Syntaxin 1, Syntaxin 2, Syntaxin 3, Syntaxin 4, Synuclein alpha, VAMP-2, Vesicular Acetylcholine Transporter/VAChT, Vesicular GABA transporter/VGAT/VIAAT, Vesicular Glutamate Transporter 1, 2, 3/VGLUT, Vesicular monoamine transporter 1 or Vesicular monoamine transporter 12, for example, present on the surface of perisynaptic regions of neurons.

In some embodiments, a targeting molecule directs a nucleic acid nanostructure (e.g., nanocapsule) specifically to cholinergic neurons. In some embodiments, a targeting molecule binds specifically to Acetylcholine/Ach, Acetylcholinesterase, Choline Acetyltransferase/ChAT, Choline transporter or Vesicular Acetylcholine Transporter/VAChT, for example, present on the surface of cholinergic neurons.

In some embodiments, a targeting molecule directs a nucleic acid nanostructure (e.g., nanocapsule) specifically to dopaminergic neurons. In some embodiments, a targeting molecule binds specifically to Adrenaline, Dopamine, Dopamine Beta Hydroxylase/DBH, Dopamine Transporter/DAT, L-DOPA, Nitric Oxide-Dopamine, Norepinephrine, Norepinephrine Transporter/NET, Parkin, Tyrosine Hydroxylase/TH or TorsinA, for example, present on the surface of dopaminergic neurons.

In some embodiments, a targeting molecule directs a nucleic acid nanostructure (e.g., nanocapsule) specifically to serotonergic neurons. In some embodiments, a targeting molecule binds specifically to DL-5-Hydroxytryptophan, Serotonin, Serotonin Transporter/SERT or Tryptophan Hydroxylase, for example, present on the surface of serotonergic neurons.

In some embodiments, a targeting molecule directs a nucleic acid nanostructure (e.g., nanocapsule) specifically to GABAergic neurons. In some embodiments, a targeting molecule binds specifically to DARPP-32, GABA, GABA Transporters 1, GABA Transporters 2, GABA Transporters 3, Glutamate Decarboxylase/GAD or Vesicular GABA transporter/VGAT/VIAAT, for example, present on the surface of GABAergic neurons.

In some embodiments, a targeting molecule directs a nucleic acid nanostructure (e.g., nanocapsule) specifically to glutamatergic neurons. In some embodiments, a targeting molecule binds specifically to Glutamate, Glutamate Transporter, Glutamine, Glutamine Synthetase, Vesicular Glutamate Transporter 1, Vesicular Glutamate Transporter 2 and/or Vesicular Glutamate Transporter 3, for example, present on the surface of glutamatergic neurons.

It should be understood that a nucleic acid nanostructure (e.g., nanocapsule) of the present disclosure may have one or more than one (e.g., a combination of different) targeting molecules.

In some embodiments, a nucleic acid nanostructure (e.g., nanocapsule) comprises a targeting molecule, an antigen and an adjuvant. For example, a nucleic acid nanostructure (e.g., nanocapsule) may comprise a single chain antibody fragments (scFv) that binds specifically to DEC205, an antigen (e.g., peptide antigen), and CpG oligonucleotides.

When nucleic acid nanostructures of the present disclosure are used in vivo, they can be administered to virtually any subject type that is likely to benefit prophylactically, therapeutically or prognostically from the delivery of nucleic acid nanostructures as contemplated herein.

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example mammals (e.g., primates (e.g., cynomolgus monkeys, rhesus monkeys), including commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In some embodiments, the non-human animal is a mammal. The non-human animal may be a male or female and at any stage of development. A non-human animal may be a transgenic animal.

In some embodiments, subjects to whom nucleic acid nanostructures are administered may have or may be at risk of developing condition that can be diagnosed or that can benefit or that can be prevented from systemic or localized delivery of one or more particular agents. Such conditions include cancer (e.g., solid tumor cancers), infections (e.g., particularly infections localized to particular regions or tissues in the body), autoimmune disorders, allergies or allergic conditions, asthma, transplant rejection, diabetes and/or heart disease.

In some embodiments, agents are delivered to prevent the onset of a condition whether or not such condition is considered a disorder.

In some embodiments, subjects may be in need of an implant or may have already received an implant, and nucleic acid nanostructures of the present disclosure are to be used in conjunction with such implant therapy.

Nucleic acid nanostructures (e.g., nanocapsules) and compositions containing nucleic acid nanocapsules may be administered to a subject (e.g., a human or non-human subject) subcutaneously or intravenously (e.g., single/multiple injection(s) or continuous infusion), or by other means.

In some embodiments, nucleic acid nanocapsules are administered to a subject as a component of a polymeric gel composition. The polymeric gel composition may be biocompatible and/or biodegradable. In some embodiments, the polymeric gel composition is formed from, and/or comprises at least one polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon-caprolactone, polyanhydrides; polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers (see, e.g., International Publication No. WO2009102465).

In some embodiments, the present disclosure provides methods for manipulating, directly in the body, dendritic-cell recruitment and activation. Immature dendritic cells patrol peripheral tissues, and on uptake of foreign substances (e.g., antigen), they may mature to express on their surface molecules (e.g., the receptor CCR7 and major histocompatibility complex (MHC) antigen) to facilitate lymph-node homing and subsequent antigen presentation to T-cells, respectively. Elements of infection that mobilize and activate dendritic cells include inflammatory cytokines, and “danger signals” related specifically to the infectious agent. Cytosine-guanosine oligonucleotide (CpG-ODN) sequences are uniquely expressed in bacterial DNA, and are potent danger signals that stimulate mammalian dendritic-cell activation and dendritic-cell trafficking. Thus, in some embodiments, the present disclosure provides methods for administering to a subject nucleic acid nanocapsules that comprise antigen (e.g., cancer antigen) and danger signals (e.g., CpG oligonucleotides).

Some aspects of the present disclosure provide compositions that comprise any one or more nucleic acid nanostructures subsaturated with polyamine polymers, or a combination of polyamine polymers and PEI-PEG copolymers. Compositions provided herein, in some embodiments, comprise a solution that contains physiological levels of salt. For example, a solution may comprise 0.1 mM to 0.9 mM magnesium (Mg²⁺) (e.g., 0.1 mM, 0.2 mM, 0.3 mM, 0.4 mM, 0.5 mM, 0.6 mM, 0.7 mM, 0.8 mM or 0.9 mM Mg²⁺). In some embodiments, a solution comprises (or further comprise) 0.5 mM to 1.5 mM calcium (Ca²⁺). For example, a solution may comprise 0.5 mM, 0.6 mM, 0.7 mM, 0.8 mM, 0.9 mM, 1.0 mM, 1.1 mM, 1.2 mM, 1.3 mM, 1.4 mM or 1.5 mM Ca²⁺.

The present disclosure provides pharmaceutical compositions. Pharmaceutical compositions are sterile compositions that comprise nucleic acid nanostructures that comprise (e.g., are subsaturated with) polyamine polymers, or a combination of polyamine polymers and PEI-PEG copolymers, and, in some embodiments, agent(s). In some embodiments, pharmaceutical compositions comprise a pharmaceutically-acceptable carrier. A pharmaceutically-acceptable carrier facilitates administration of the nucleic acid nanostructures.

Nucleic acid nanostructures, when delivered systemically, may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients.

Nucleic acid nanostructures of the present disclosure (including compositions comprising the nanostructures) may be used in a variety of applications, including biomedical applications such as nanomedicine. For example, nucleic acid nanostructures may be used for drug delivery (e.g., targeted drug delivery), immunotherapy, diagnostics and molecular biology (for a review, see, e.g., Smith D. et al. Nanomedicine (Lond). 2013 January; 8(1):105-21, incorporated by reference herein). In some embodiments, nucleic acid nanostructures may be used as scaffold-based biosensors (see, e.g., Pei H. et al. NPG Asia Materials (2013) 5, 1-8, incorporated by reference herein)

Nucleic acid nanostructures of the present disclosure may be used to investigate cellular mechanism, or they may be use in the field of material sciences. For example, the present disclosure contemplates the assembly of chiral plasmonic nanostructures with tailored optical response (see, e.g., Liedl et al. 2012 Nature, 483, 311, Incorporated by Reference herein), assembly of anistropic plasmonic nanostructures (see, e.g., Pal et al. 2011 J. Am. Chem. Soc. 133, 17606-17609, incorporated by reference herein), and layer-by-layer growth of superparamagnetic and fluorescently barcoded nanostructures (see, e.g., Wang et al. 2007 Nanotechnology 18, 40, 405026, incorporated by reference herein).

### EXAMPLES

### Example 1

To investigate the effect of spermine polymers on the structural integrity of DNA nanostructures in low-magnesium buffers, DNA nanostructures (schematized in FIG. 1) were incubated with spermine polymers, dialyzed and analyzed by gel electrophoresis as follows. 100 L of 1 nanomolar (nM) DNA nanostructure in 0.5×TE buffer (5 mM Tris, 1 mM ethylenediaminetetraacetic acid (EDTA), pH 7.4) supplemented with 6 mM Mg²⁺+/−60 micromolar (μM) spermine polymers were dialyzed in 1 L of 0.5×TE buffer containing either 0 or 0.6 millimolar (mM) Mg²⁺ for 18 hours. After dialysis, the samples were loaded onto a 2% agarose gel composed only of 0.5×TBE (Tris/Borate/EDTA) and 11 mM Mg²⁺. As shown in FIG. 1, no noticeable differences were observed in the gel between the control and the spermine-incubated samples; however, transmission electron microscopy (TEM; stained with 2% uranyl formate) analysis shows that the control structure was denatured at both 0 and 0.6 mM Mg²⁺ concentration, while nanostructures incubated with spermine polymers maintained structural integrity in buffers containing 0.6 mM Mg²⁺.

### Example 2

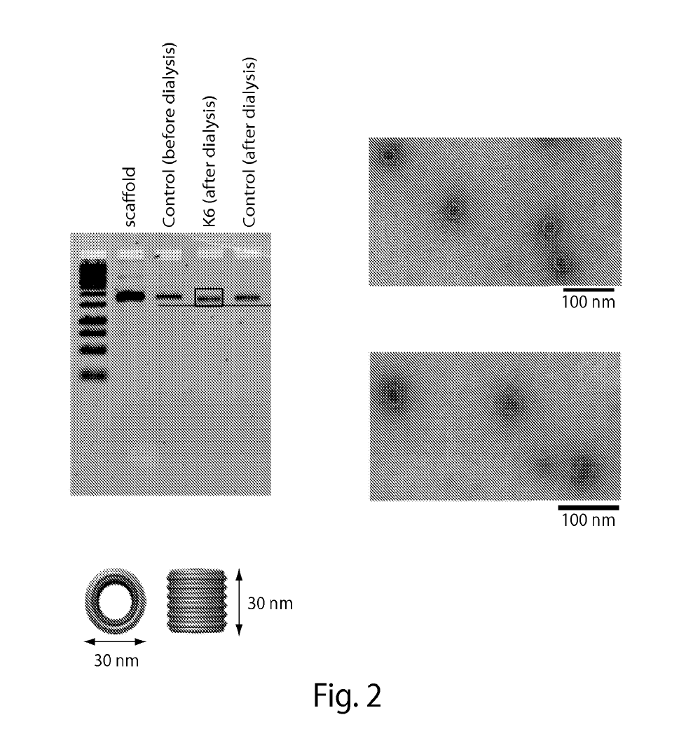

To investigate the effect of polylysine polymers on the structural integrity of DNA nanostructures in no-magnesium buffers, DNA nanostructures were incubated with polylysine polymers, dialyzed and analyzed by gel electrophoresis as follows. 100 L of 1 nM DNA nanostructure in 0.5×TE buffer supplemented with 6 mM Mg²⁺+/−60 M polylysine polymer (K6=KKKKKK) were dialyzed in 1 L of 1×PBS (10 mM Na₂HPO₄, 137 mM NaCl, pH 7.4) containing 0 Mg²⁺ for 18 hours. After dialysis, the sample were loaded onto a 2% agarose gel composed only of 0.5×TBE and 11 mM Mg²⁺. As shown in FIG. 2, no noticeable differences were observed in the gel between the control and the polylysine polymer-incubated samples; however, TEM analysis shows that the control structure was completely denatured at 0 mM Mg²⁺ concentration, while structures incubated with polylysine polymers maintained structural integrity.

### Example 3

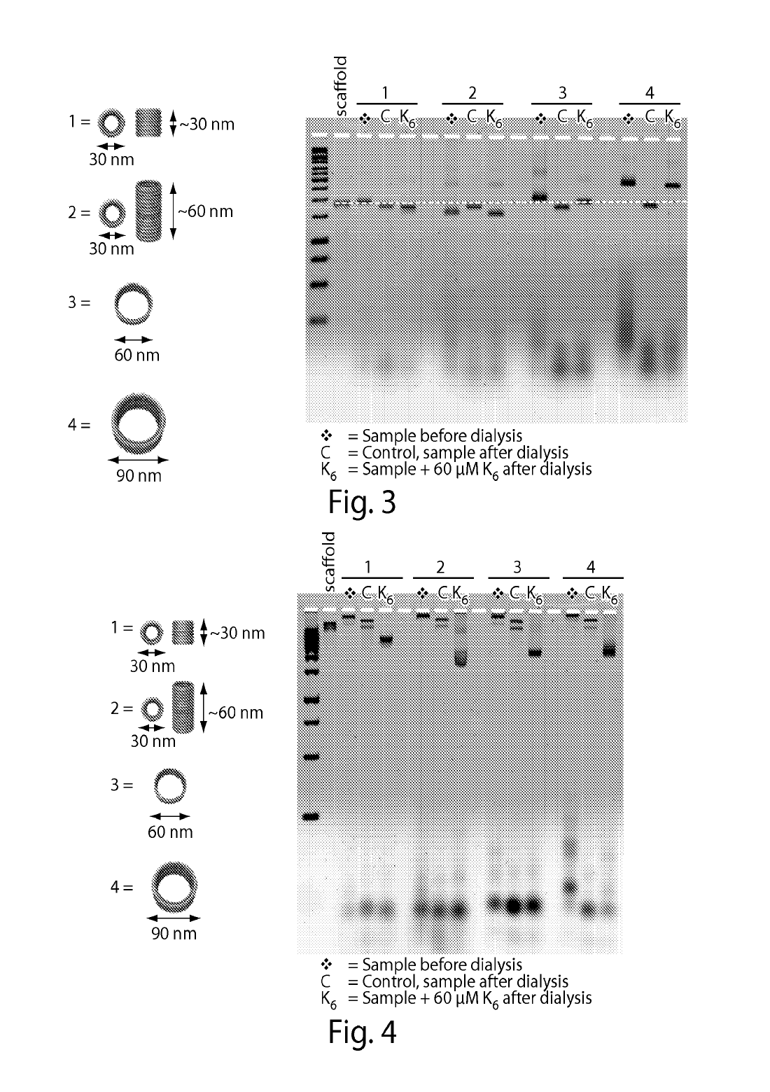

To investigate the effect of polylysine polymers on the structural integrity of DNA nanostructures of various dimensions in the presence of magnesium, DNA nanostructures of different lengths and diameters (representative schematics 1-4 in FIG. 3) were incubated with polylysine polymers, dialyzed and analyzed by gel electrophoresis as follows. 100 μL of 1 nM DNA nanostructure in 0.5×TE buffer supplemented with 6 mM Mg²⁺+/−60 μM polylysine (KKKKKK=K6) were dialyzed in 1 L of 0.5×TE for 18 hours. After dialysis, the samples were loaded onto a 2% agarose gel that included 0.5×TBE and 11 mM Mg²⁺. In FIG. 3, the dashed line indicates the mobility of the naked DNA scaffold in the gel. The control sample in all cases was unfolded into the naked scaffold on dialysis. In contrast, the polylysine-coated structures maintained their integrity and mobility.

### Example 4

To investigate the effect of polylysine polymers on the structural integrity of DNA nanostructures of various dimensions in the absence of magnesium, DNA nanostructures of different lengths and diameters (representative schematics 1-4 in FIG. 4) were incubated with polylysine polymers, dialyzed and analyzed by gel electrophoresis as follows. 100 L of 1 nM DNA nanostructure in 0.5×TE buffer supplemented with 6 mM Mg²⁺+/−60 μM polylysine (KKKKKK=K6) were dialyzed in 1 L of 0.5×TE for 18 hours. After dialysis, the samples were loaded onto a 2% agarose gel that included only 0.5×TBE without Mg²⁺. As shown in FIG. 4, only the polylysine polymer-coated nanostructures showed mobility on this gel.

### Example 5

To assess polylysine polymer-induced stability of DNA nanostructures in low-magnesium buffers, DNA nanostructures of various dimensions (representative schematics 1-4 in FIG. 5) were incubated with polylysine polymers, dialyzed and imaged by TEM. 100 μL of 1 nM DNA nanostructure in 0.5×TE buffer supplemented with 6 mM Mg²⁺+/−60 μM polylysine polymer (KKKKKK=K6) were dialyzed in 1 L of 0.5×TE buffer for 18 hours. FIG. 5 shows TEM images of raw samples, stained with 2% uranyl formate. The control sample in all the cases was unfolded into the naked scaffold on dialysis. In contrast, the polylysine polymer-coated structures maintained their structural integrity.

### Example 6

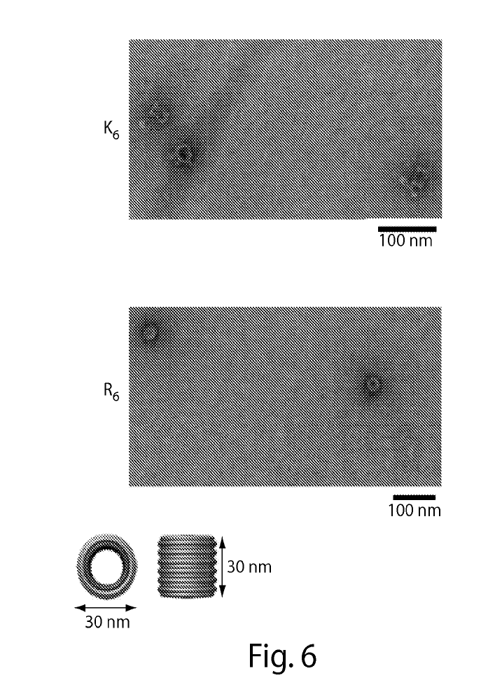

To compare the effects of polylysine polymers and polyarginine polymers on the structural integrity of DNA nanostructures, DNA nanostructures (schematized in FIG. 6) were incubated with polylysine or polyarginine polymers, dialyzed and imaged by TEM as follows. 100 μL of 1 nM DNA nanostructure in 0.5×TE buffer supplemented with 6 mM Mg²⁺ and 60 μM polylysine polymer (KKKKKK=K6) or 60 μM polyarginine polymer (RRRRRR=R6) were dialyzed in 1 L of 1 xPBS for 18 hours. After dialysis, the samples were imaged by TEM. As shown in FIG. 6, DNA nanostructures incubated with polylysine polymers aggregated less and exhibited better structural integrity relative to those incubated with polyarginine polymers.

### Example 7

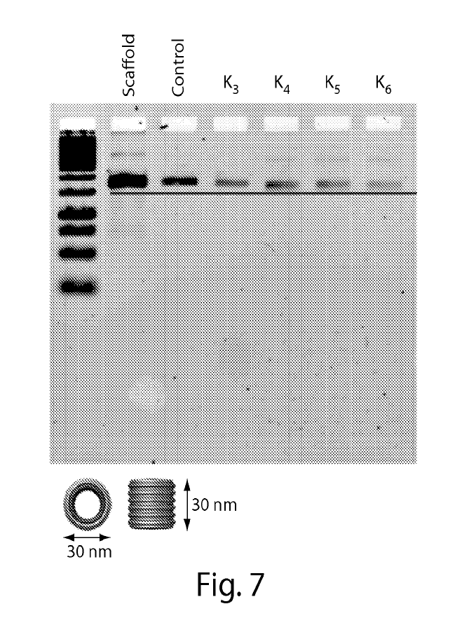

To compare the effects of different lengths of polylysine polymers on the structural integrity of DNA nanostructures, DNA nanostructures (schematized in FIG. 7) were incubated with polylysine polymers, dialyzed and analyzed by gel electrophoresis as follows. 100 μL of 1 nM DNA nanostructure in 0.5×TE buffer supplemented with 6 mM Mg²⁺ and 60 μM polylysine polymers of various lengths (e.g., K3, K4, K5 and K6) were dialyzed in 1 L of 1×PBS for 18 hours. After dialysis, the samples were loaded onto a 2% agarose gel that included only 0.5×TBE and 11 mM Mg²⁺. As shown in FIG. 7, the structures coated with polylysine polymers with five lysines (K5) and polylysine polymers with six lysines K6 exhibited slightly faster mobility on the gel, indicative of a better folding. The nanostructures also showed higher structural integrity when imaged in TEM (not shown).

### Example 8

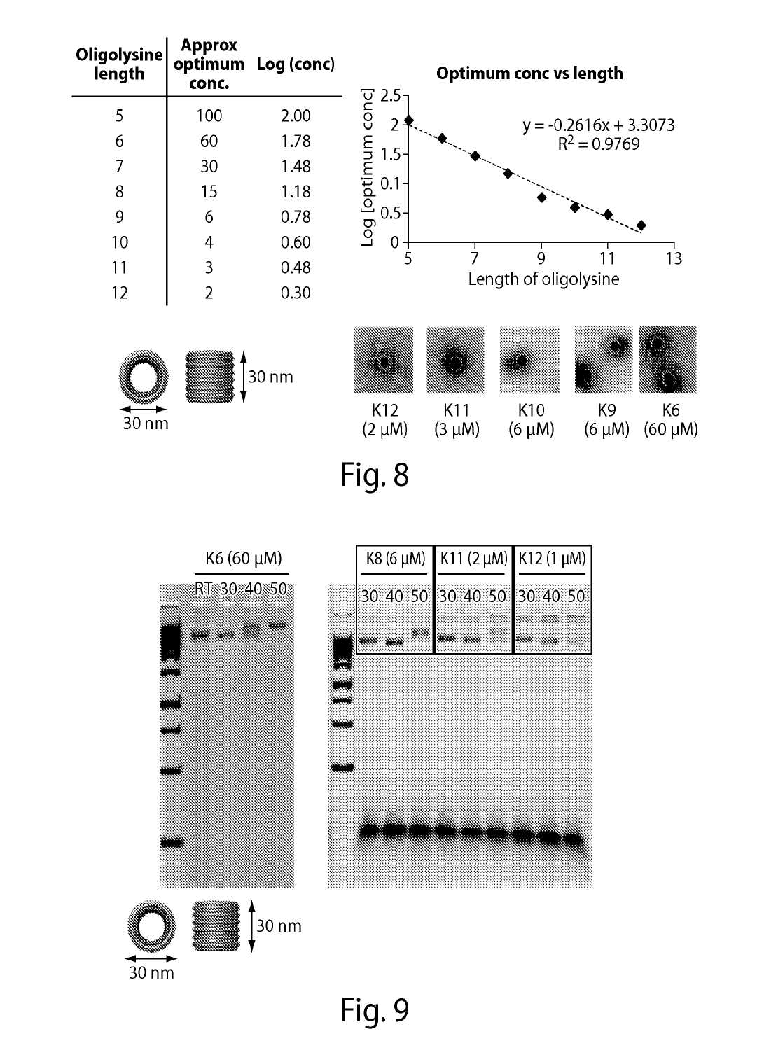

To compare the effects of polylysine polymer length on respective polylysine polymer concentrations required to maintain the structural integrity of DNA nanostructures, DNA nanostructures (schematized in FIG. 8) were incubated with polylysine polymers of various lengths and concentrations, dialyzed and imaged by TEM as follows. 100 L of 1 nM DNA nanostructure in 0.5×TE buffer supplemented with 6 mM Mg²⁺ and various concentrations of polylysine polymers of different lengths (e.g., K5, K6, K7, K8, K9, K10, K11 and K12) were dialyzed in 1 L of 1×PBS for 18 hours. As shown in FIG. 8, lower concentrations of longer polylysine polymers stabilized the DNA nanostructures.

### Example 9

To compare the effects of polylysine polymer length with respect to thermal stability of DNA nanostructures, DNA nanostructures (schematized in FIG. 9) were incubated with polylysine polymers of various lengths, dialyzed, incubated at different temperatures and analyzed be gel electrophoresis as follows. 20 μL of polylysine polymer (e.g., K6, K8, K11, K12)-coated DNA nanostructures were dialyzed in 1×PBS for 18 hours and then incubated at different temperatures (e.g., 30° C., 40° C. or 50° C.) for 24 hours. The samples were then loaded onto a 2% agarose gel that included 0.5×TBE without Mg²⁺. FIG. 9 shows a decrease in mobility of the DNA bands, which represents denaturation of the nanostructures. The decrease in mobility was observed at 40° C. for K6, while no change in mobility was observed even at 50° C. for structures coated with K12. Thus, longer polylysine polymers appear to provide enhanced thermal stability relative to shorter polymers.

### Example 10

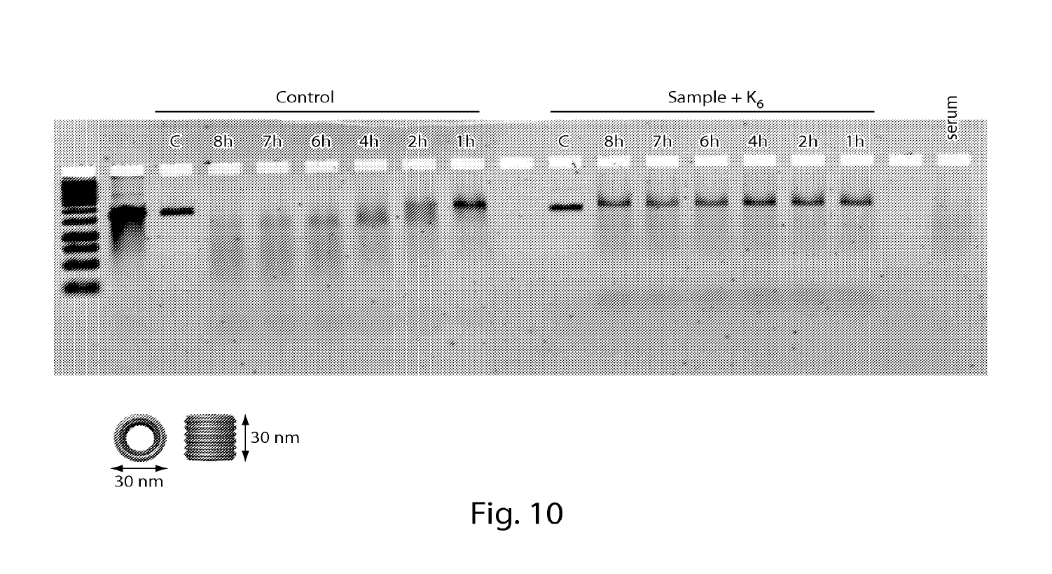

To compare the effects of polylysine polymer with respect to nuclease stability of DNA nanostructures, DNA nanostructures (schematized in FIG. 10) were incubated with polylysine polymers, dialyzed, incubated in fresh cell media for different periods of time, and analyzed be gel electrophoresis as follows. 20 L of polylysine polymer (K6)-coated DNA nanostructures, dialyzed into 1×PBS for 18 hours, was incubated with 10 L of fresh cell medium (10% FBS in GIBCO® RPMI-1640 media) at 37° C. different periods of time. The samples were then loaded onto a 2% agarose gel that included 0.5×TBE and 11 mM Mg²⁺. FIG. 10 shows a decrease in intensity of gel bands, which is representative of degradation of DNA nanostructure by nucleases. The control sample was degraded within 1 hour of incubation, while the structural integrity of the sample coated with polylysine polymers with six lysines (K6) were maintained for 8 hours of incubation with fresh cell medium.

### Example 11

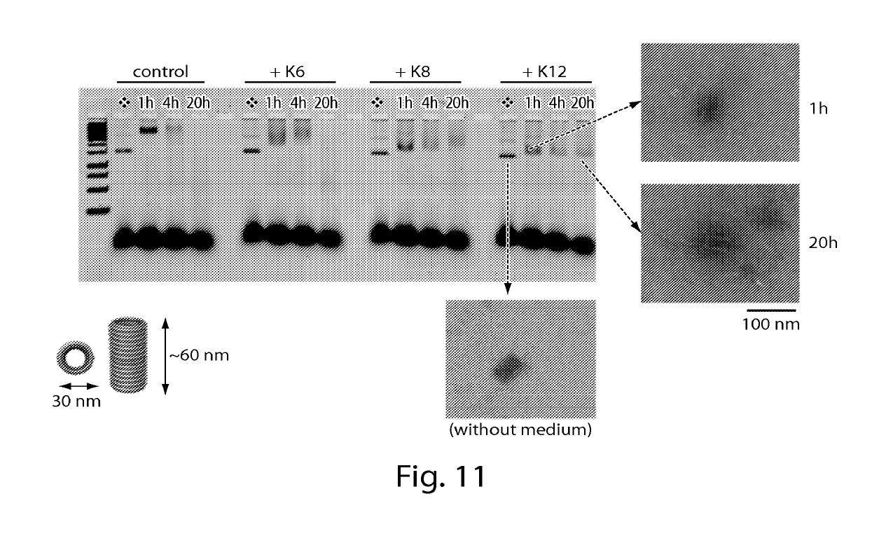

To compare the effects of polylysine polymer length with respect to nuclease stability of DNA nanostructures in fresh cell culture media, DNA nanostructures (schematized in FIG. 11) were incubated with polylysine polymers of different lengths, dialyzed, incubated in fresh cell media for different periods of time, and analyzed be gel electrophoresis as follows. 20 μL of polylysine (e.g., K6, K8, K12)-coated DNA nanostructures, dialyzed into 1×PBS for 18 hours, was incubated with 10 μL of fresh cell medium (10% fetal bovine serum (FBS) in GIBCO® RPMI-1640 media) at 37° C. for different periods of time. The samples were then loaded onto a 2% agarose gel that included 0.5×TBE and 11 mM Mg²⁺. FIG. 11 shows a decrease in intensity of gel bands, which is representative of degradation of DNA nanostructure by nucleases. The control sample was degraded within 1 hour of incubation, while the structural integrity of the sample coated with polylysine polymers with twelve lysines (K12) were maintained for 24 hours of incubation with fresh cell medium. TEM imaging of the serum incubated nanostructures showed intact DNA nanostructures. Longer polylysine polymers appeared to impart higher/longer stability to the DNA nanostructures in fresh cell medium.

### Example 12

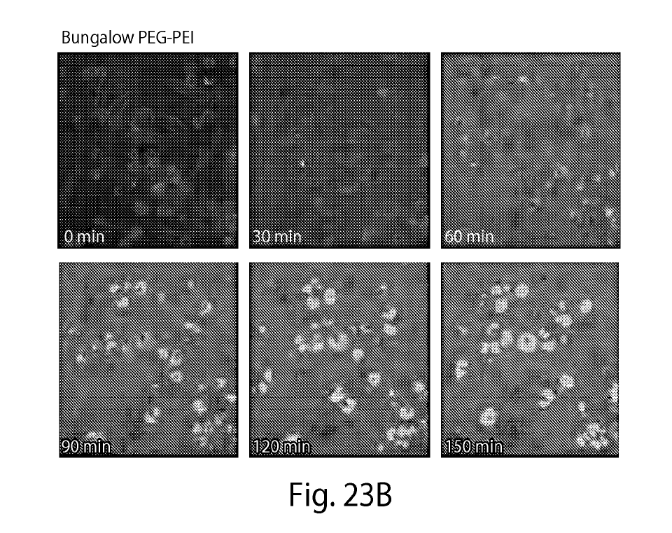

DNA nanostructures were coated with PEI-PEG copolymers. FIG. 23A shows the ligation scheme of the PEG-NHS coupling to the PEI primary amines. Reaction conditions were PBS pH 8, and overnight incubation at room temperature. FIG. 23A also shows an agarose gel analysis of DNA nanostructure stability when incubated for various lengths of time in serum-active cell medium. FIG. 23B shows uptake of PEI-PEG-coated DNA nanostructures in bone marrow-derived dendritic cells (BMDC). Panels are 30 minutes apart and show the uptake of Cy5 labeled DNA nanostructure inside the cells.

### Example 13

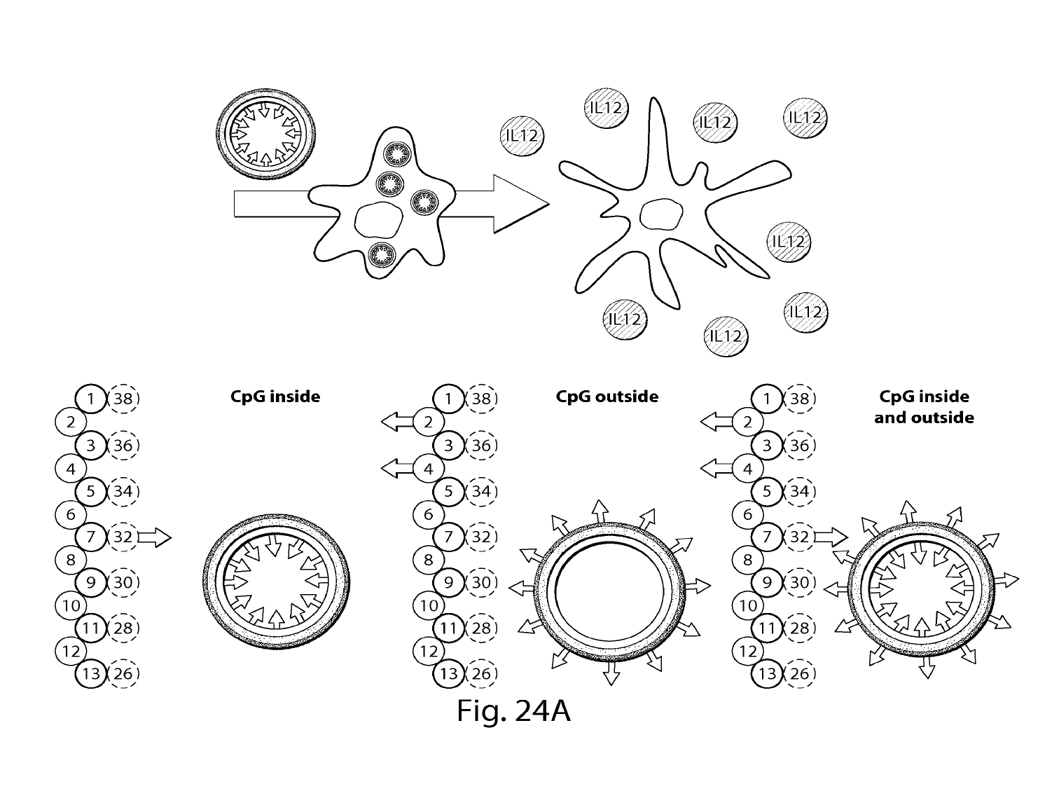

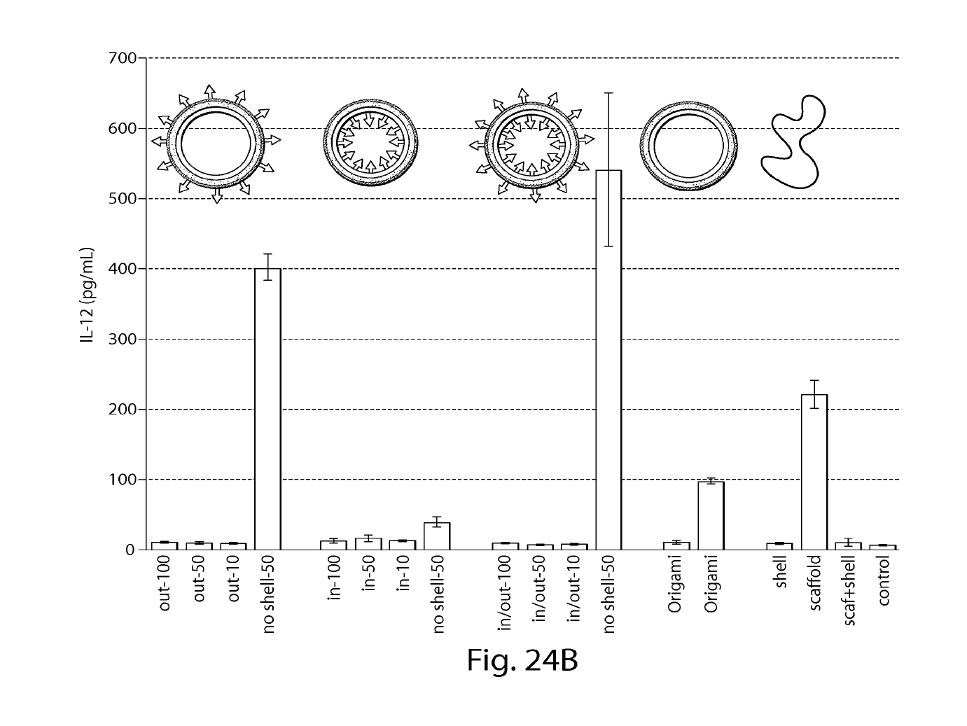

FIG. 24A shows a schematic outline of a TLR9 activation experiment. DNA origami structures were loaded with CpG danger signals in different locations, e.g., interior and/or exterior of nanocapsules. Immature dendritic cells were stimulated with the CpG loaded nanostructures and allowed to mature overnight. When TLR9 was stimulated by CpG, IL12 cytokine was produced and secreted. FIG. 24B shows an IL12 cytokine ELISA assay to evaluate TLR9 stimulation. Major activation was observed when the CpG danger signals were placed on the outside of the DNA nanostructure. However, when a protective coating was present, the immunogenic activity was cloaked. This demonstrates structural integrity of the DNA nanostructure over the time course of uptake and endosomal residence, even without the protective shell.

### Example 14

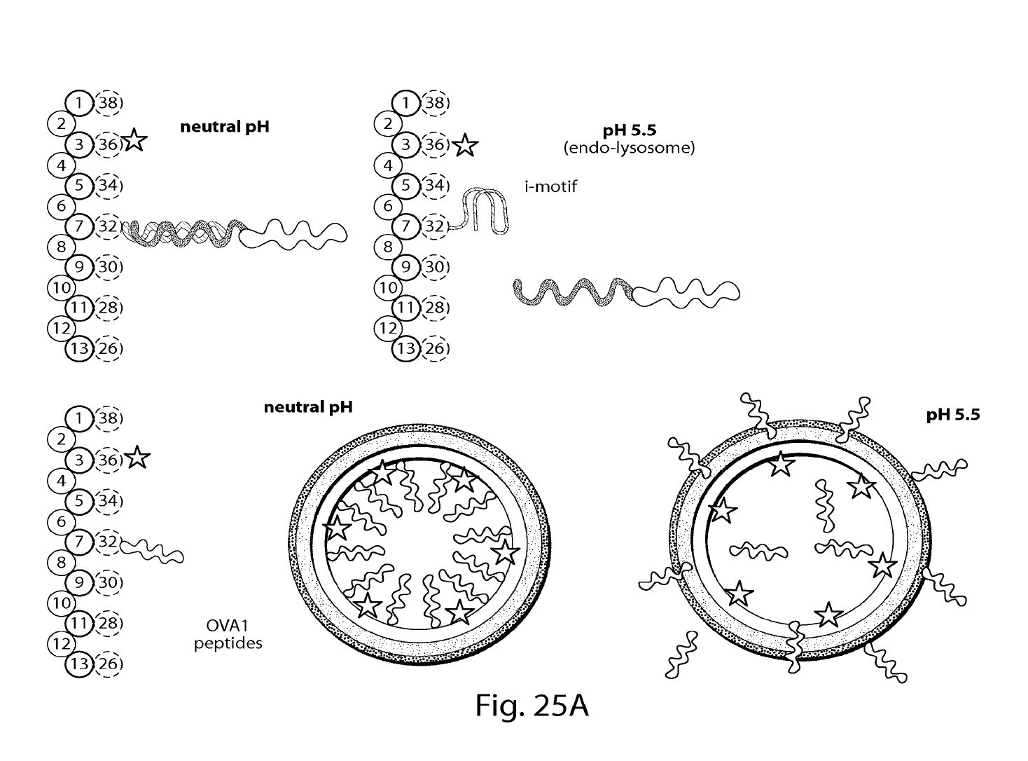

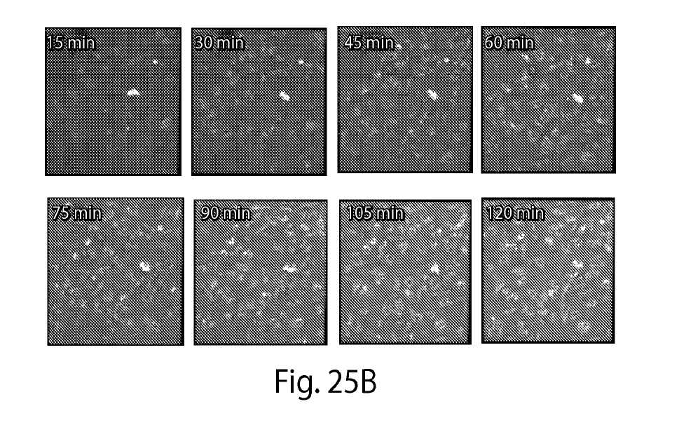

FIG. 25B shows confocal imaging of peptide antigen release in live cells. BMDC were incubated with DNA nanostructures loaded with antigen cargo. Panels are 15 minutes apart and show an initial colocalization during the uptake process, followed by a clear separation of the 2 channels, where the antigen peptide remains in the cell and travels to the cell membrane where it is presented on MHC receptors, the DNA nanostructure is excreted from the cells.

### Example 15

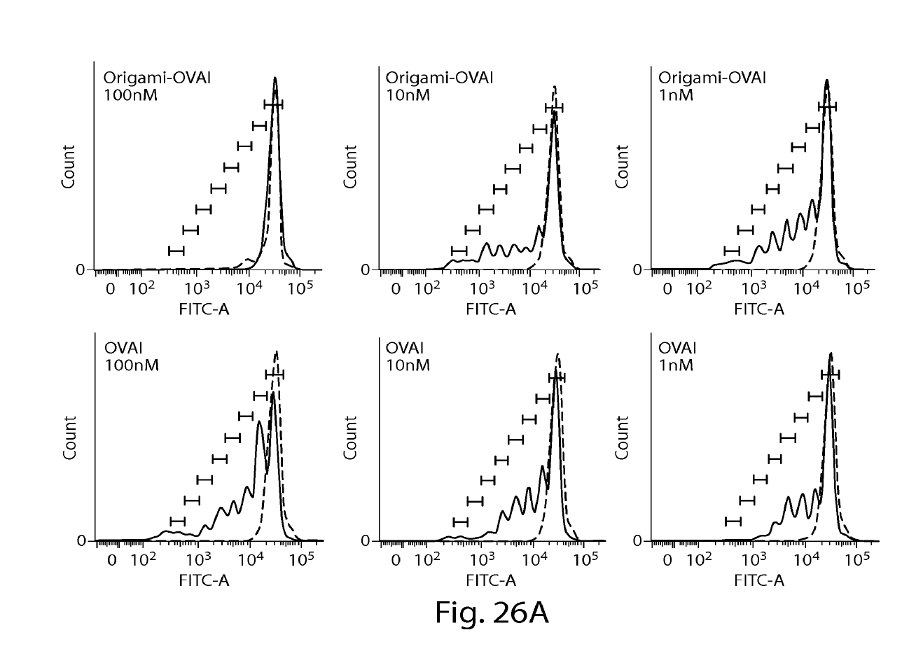

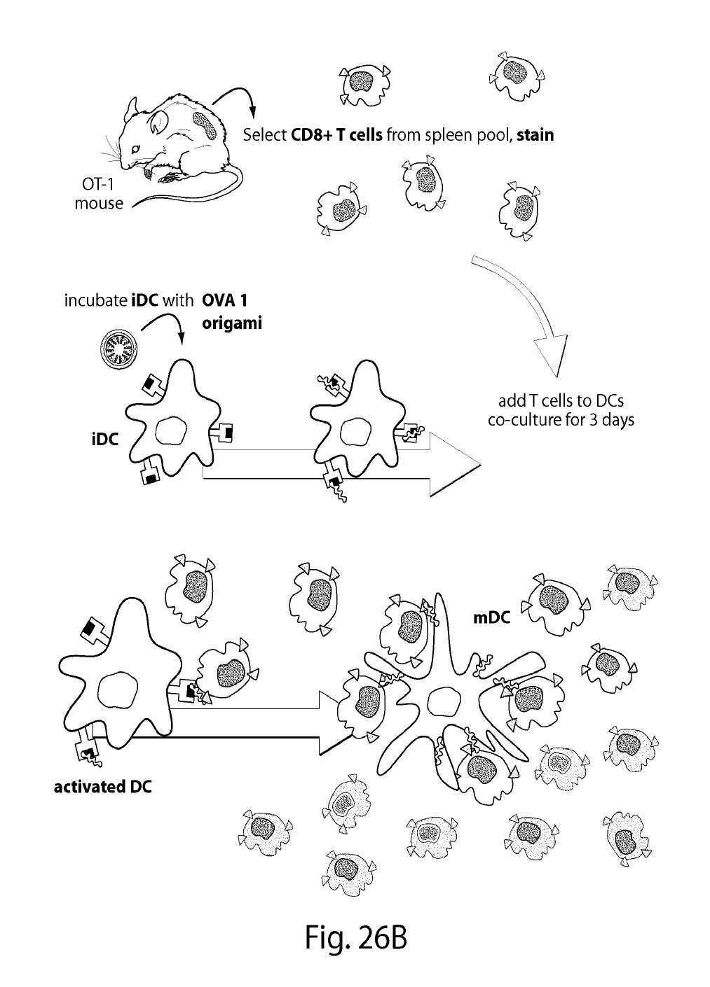

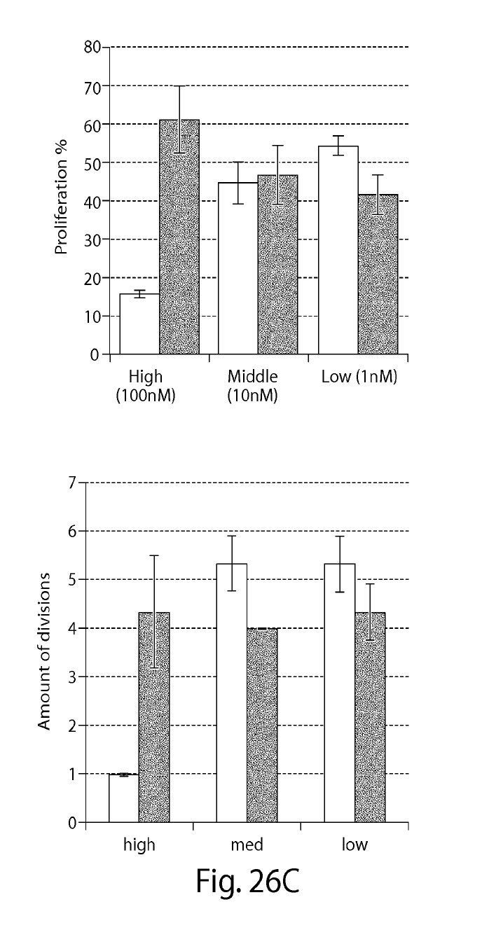

FIG. 26A shows results of a T-cell activation assay, measured by fluorescent intensity profiling with flow cytometry. BMDCs were incubated with various doses of antigen-DNA nanostructures, or antigens alone as control. Co-culture with OT-1 derived T-cells for 3 days resulted in stimulation and division of the T-cell population. The 1 nM delivery of antigen through DNA nanostructure was superior over free antigen uptake, demonstrating the strength and advantage of the methods provided herein.

FIG. 26B shows a schematic outline of a T-cell activation experiment. CD8⁺ T-cells were harvested from the spleen of an OT-1 transgenic mouse and stained green. The T-cells were co-cultured with BMDCs and DNA nanostructures loaded with peptide antigens, or antigens alone. Only when the antigens were presented by the DCs through MHC-I did the T-cells proliferate.

FIG. 26C shows quantification of the T-cell experiments using FlowJo analysis. For the high and middle dosing, there was overstimulation-induced cell death. At the lowest concentration, 1 nM, both proliferation % (top graph) and amount of divisions (bottom graph) were increased with the DNA delivery method of the present disclosure compared to free peptide delivery. Not shown are similar results for MHC-II presentation and OT-2 T cell activation.

### Example 16

The purpose of this Example is to construct DNA-origami nanocapsules and investigate which structural features are important for efficient delivery of antigens and danger-signals to dendritic cells. Nanocapsules are constructed with a range of sizes, cargos, and surface decorations, and the capacity of these particles to stimulate dendritic cells (DCs) to activate T cells is investigated. Nanocapsules that most potently lead to activated T cells in vitro and in vivo are tested for their capacity to induce antigen-specific anti-tumor immunity in vivo. The immunological assays described in Example 17 are used to test the nanoparticles described in this Example.

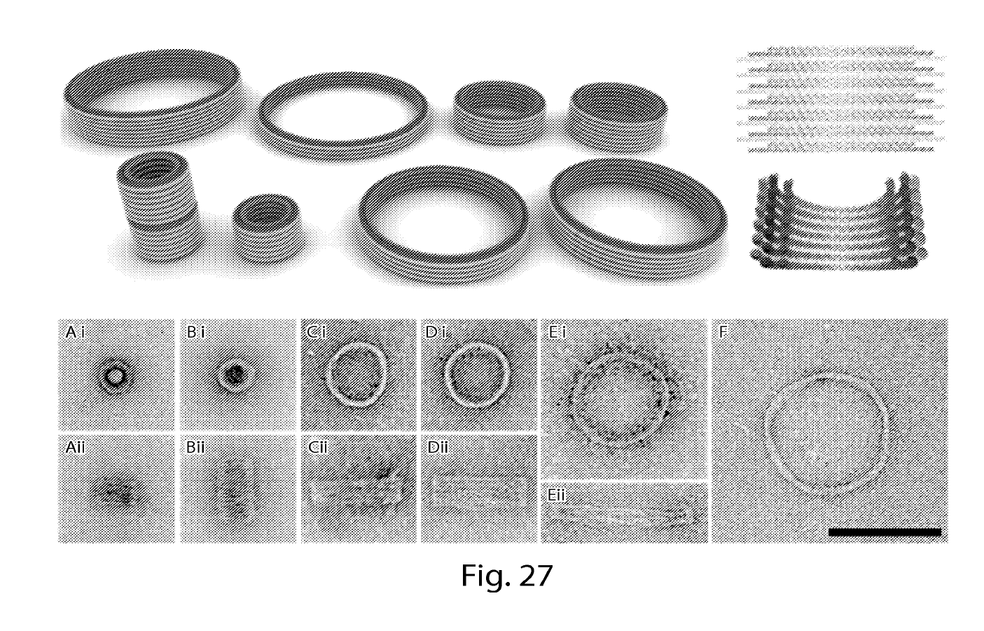

Size and shape of nanoparticles, in general, have an effect on uptake and sorting into dendritic cells (DCs). Altering the size of nanocapsules enables modulation of the number of cargo molecules loaded per nanostructure. In this Example, DNA origami is used to build nucleic acid nanocapsules. With DNA origami, a long scaffold strand is assembled with short staple strands into a sheet composed of a parallel array of double helices held together by numerous strand crossovers between helices. To produce a sheet that curves into a cylinder, a sheet having three layers (medium gray, dark gray, and light gray in FIG. 27, top right) is constructed; the inside layer (medium gray) is programmed (e.g., rationally designed) to have shorter helices and the outer layer (light gray) is programmed to have to have longer helices (Dietz, H., et al. Science 325, 725-730 (2009); Douglas, S. M. et al. Nature 459, 414-418 (2009); and Han, D. et al. Science 332, 342-346 (2011), incorporated by reference herein). This structure results in contraction on the inside layer and expansion on the outside layer. In addition, the ends of the sheet are designed to seal, which reinforces the desired curvature. The walls of the cylinder are ˜6 nm thick, and are much more rigid than a single-layer DNA-origami sheet (2.5 nm thick).

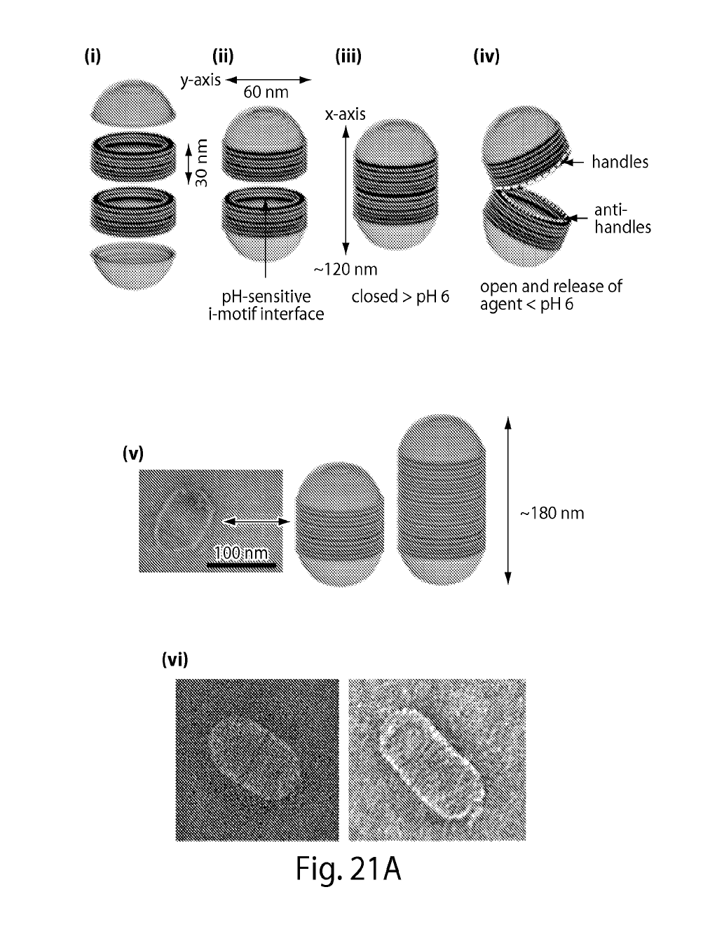

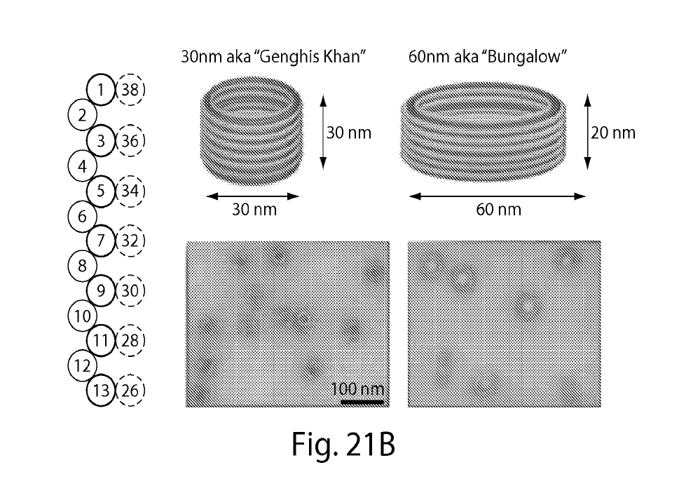

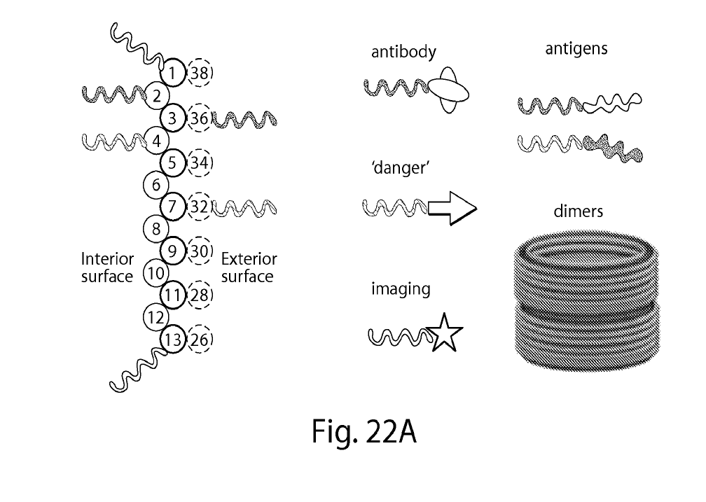

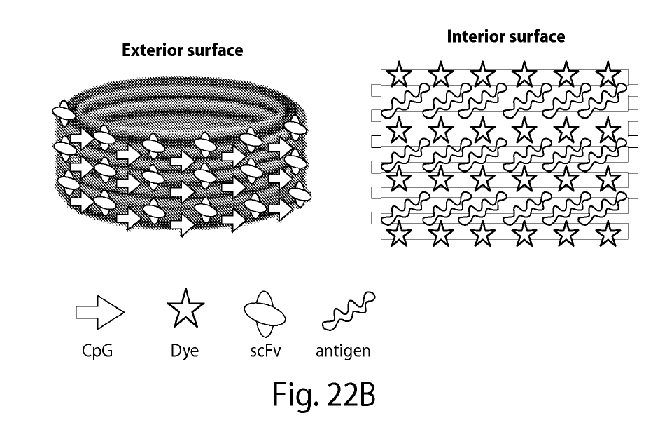

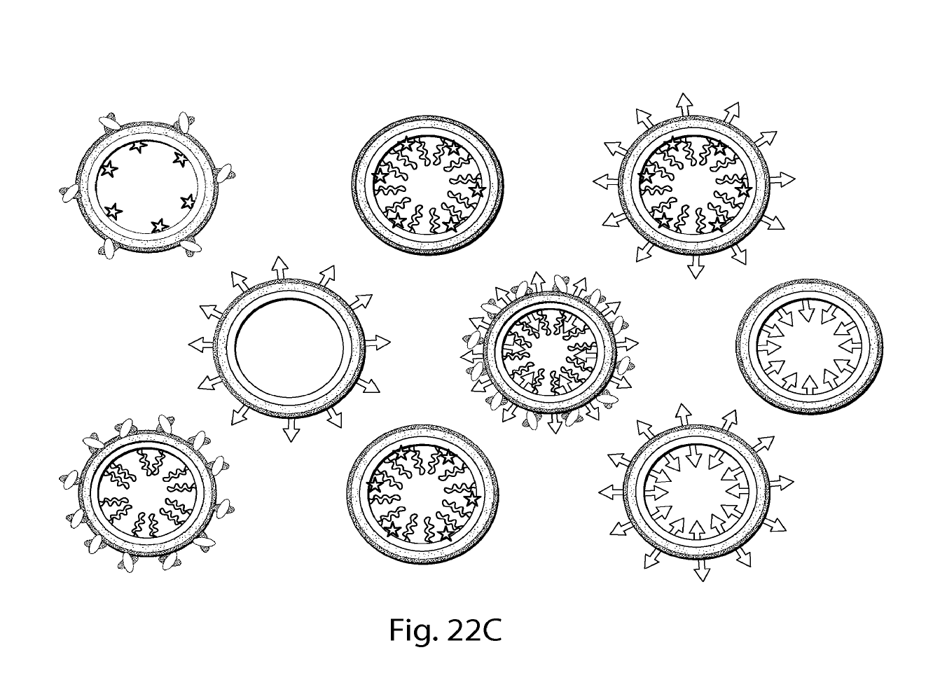

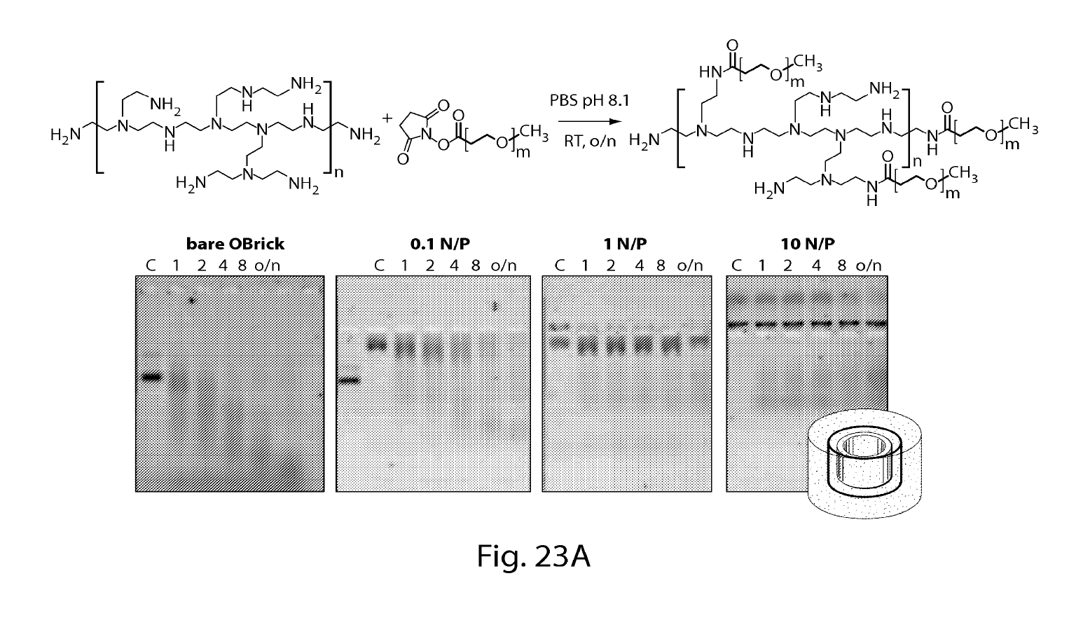

An example of a nanocapsule structure is shown in FIG. 21A(i) and contains two dome components (light gray) and a prescribed number of cylindrical components (medium gray). The architecture of the dome components retains the three layers of helices (outer, middle, inner), similar to the cylindrical components. Provided herein is a robust strategy for coaxial stacking of defined numbers of cylinder domains. Two nanocapsules were constructed, each with a diameter of 60 nm: one programmed to incorporate two cylinder domains (120 nm nanocapsule length) (FIG. 21A(vi), left panel) and one programmed to incorporate four cylinder domains (180 nm nanocapsule length) (FIG. 21A(vi), right panel).

Further, nanocapsules with diameters of 31 nm, 60 nm, or 87 nm, and with lengths of 60 nm, 120 nm, 180 nm, 240 nm, 300 nm, or 360 nm are constructed. Each version of the nanocapsule is decorated with targeting agents, as described below, to determine whether the relative shape/size dependence of uptake is altered by the inclusion versus exclusion of targeting agents. The interior cavity is decorated with three-dozen Cy5 fluorophores per particle and track the uptake into BMDCs as described in Example 17.

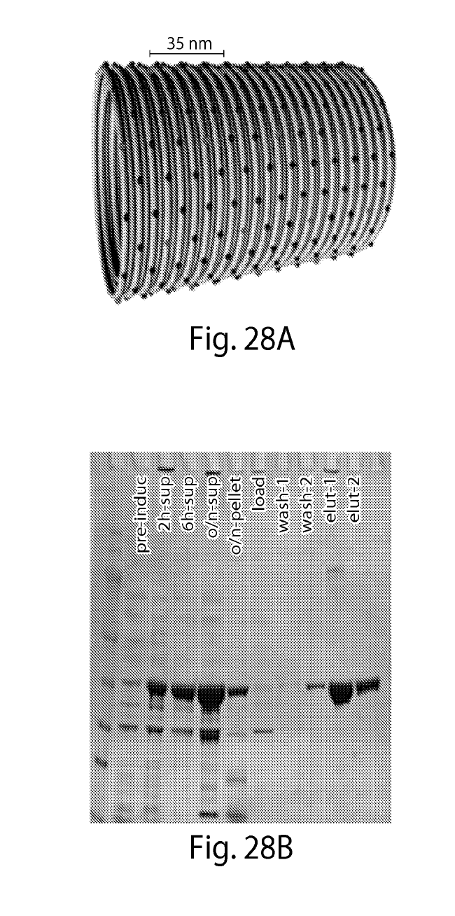

Potent per particle activation of bone marrow-derived dendritic cells (BMDCs) is investigated through display of a sparse uniform lattice of anti-DEC205 and dense uniform decoration with anti-CD40. Displaying anti-DEC205 affinity agents is expected to facilitate BMDC targeting; however, induction of DEC205 clustering is minimized to avoid BMDC inactivation. Conversely, CD40 crosslinking leads to BMDC activation, therefore a maximum density of anti-CD40 affinity agents is displayed on the outer surface of the nanocapsule to trigger CD40 crosslinking and signaling. An example of a basic framework for decorating the outside surface of nanocapsules is a rhombic lattice with spacings of 8.7 nm to the six nearest neighbors (FIG. 28, black and gray dots); each 60 nm diameter cylinder bears 84 outer-lattice positions. Each lattice position is decorated independently with custom ssDNA “handles”. Then, these lattice handles are partnered in a sequence-specific fashion with ligands that are covalently linked to ssDNA “anti-handles” with complementary sequences to the cognate handles. Spacings of anti-DEC205 that are increasing multiples of 8.7 nm are tested and the remaining lattice spots are filled with anti-CD40. FIG. 28 (gray dots) shows an example pattern with anti-DEC205 lattice points spaced 35 nm apart.

Affinity agents are used for site-specific tagging with a ssDNA anti-handle for oriented presentation after hybridization to complementary ssDNA handle strands positioned at selected positions on the rhombic-lattice organized surface. Using single chain antibody fragments expressed in Escherichia coli (E. coli), various tags (e.g., unique cysteine, aldehyde (Rabuka, D., et al. Nat Protoc 7, 1052-1067 (2012)), sortase (Popp, M. W. et al. Nat Chem Biol 3, 707-708 (2007)), ybbR (Yin, J., et al. Nat Protoc 1, 280-285 (2006)), SNAP (Hussain, A. F., et al. Curr Pharm Des 19, 5437-5442 (2013)) and/or amino terminus (Scheck, R. A., et al. J Am Chem Soc 130, 11762-11770 (2008))) are installed via site-directed mutagenesis and then evaluated. For RNA, versions of aptamers optimized for incorporation of 2′ fluoro pyrimidines (e.g., 2′fluoropyrimidine-substituted RNA aptamers that binds to DEC205 (Wengerter, B. C. et al. Mol Ther 22, 1375-1387 (2014)) are used for increased stability relative to unmodified RNA. Although the affinity of individual scFvs or aptamers may not be high (nanomolar to micromolar), many copies are displayed on the surface with prescribed spacing to achieve multivalent avidity.

A basic framework for decorating the inside of the nanocapsule is a rhombic lattice with a similar spacing to that found on the outside of the nanocapsule, with 84 lattice positions per 60 nm diameter cylinder domain. Similarly, each position is decorated with a ssDNA handle that can be addressed by a complementary ssDNA anti-handle covalently linked to desired cargo molecules. CpG and poly I:C are used as danger signals, and OVA1 and OVA2 as model antigens. In order to investigate the importance of induced dimerization of TLRs and double the loading capacity per nanocapsule, a tethered pair of danger signals is attached to each lattice position, via an anti-handle-danger signal that has an internal thiol that can be conjugated to a maleimide-bearing danger signal, to create a branched molecule. These danger signals are programmed to be released from the inner wall of the nanocapsules in response to an acidic pH (e.g., below 5.5) by encoding the sequence of the handle to be cytosine-rich such that it will preferentially form an i-motif ssDNA structure once its cytosines are protonated on their N-3 (Dong, Y., et al. Acc Chem Res 47, 1853-1860 (2014)).

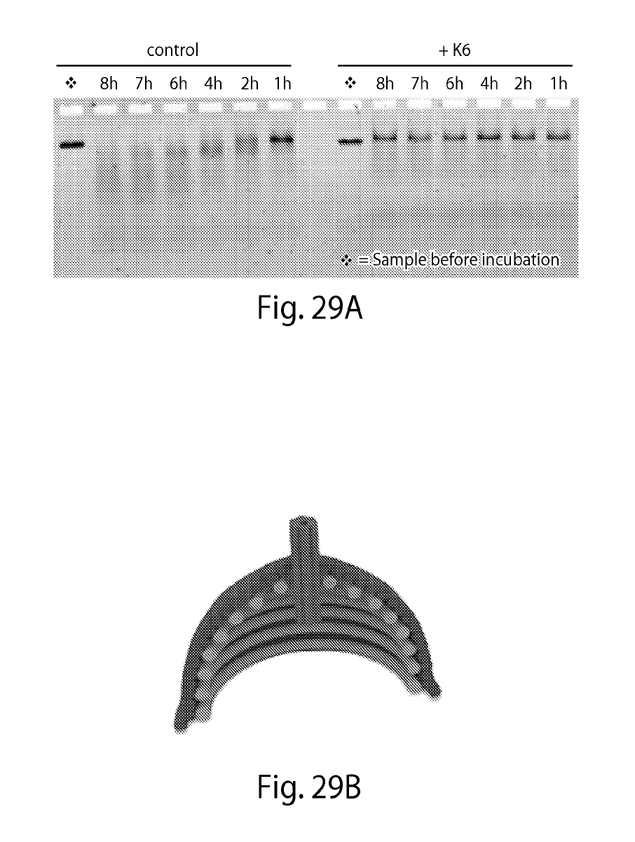

The nanocapsules are designed to protect the cargo from degradation prior to arrival into an endosomal compartment. The walls of the nanocapsules contain packed double helices gaps no larger than 1 nm. The top of the domes contain a 8 nm hole, which is filled with a self-assembled DNA six-helix bundle plug, such that no macromolecule larger than 1 nm can diffuse in or out (FIG. 29, right). In order to protect the nanocapsule itself from degradation by nucleases or low-salt denaturation, it is coated with a shell composed of oligolysines (FIG. 29, left).

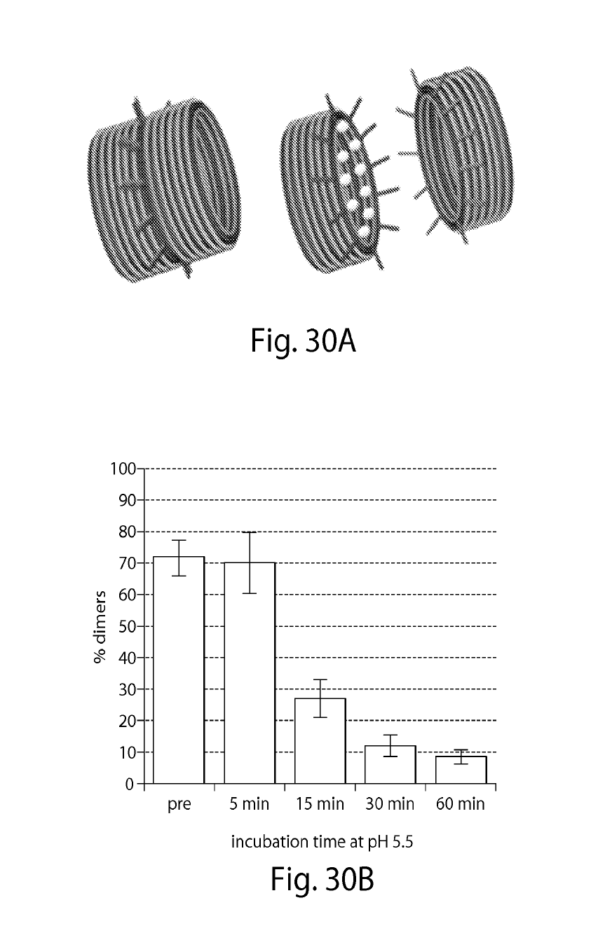

Upon sensing a pH of 5.5 or lower—the pH of endosomes in an immature BMDC—the nanocapsule open up. Furthermore, the anchors connecting the cargo to the inner wall of the nanocapsule release below pH 5.5. To achieve this acidic pH trigger, the i-motif is used, which is a cytosine rich sequence that dissociates from a Watson-Crick base-paired partner strand and then fold up on itself upon protonation of the N-3 of the cytosine ring (Dong, Y., et al. Acc Chem Res 47, 1853-1860 (2014)). An example of this strategy is shown in FIG. 30, where the base pairing between strands that hold together the two cylinder domains is programmed to be disrupted at pH 5.5 by formation of ssDNA i-motif structures within cytosine-rich sequences. Other pH triggers are programmed with the connecting helices pointing to the inside of the nanocapsule, to make it more difficult for nucleases to cleave these and prematurely trigger nanocapsule opening.

DNA nanocapsules of the present disclosure are well-tolerated by BMDCs with no toxic effects nor altered viability and capable of inducing IL-12 secretion and of inducing them to activate T cells in a fashion specific to the antigens presented by the nanocapsules. BMDCs were incubated with 5 nM DNA nanocylinders labeled with a Cy5 reporter dye and imaged after one hour. Accumulation of the DNA origami inside the cells was visible. Nuclei were stained with Hoechst and bright field images showed no changes in cell phenotype, hence, no toxic effects of the DNA sample to the cells.

In some instances, oligoamine coatings applied to protect the nanocapsules may inhibit, to a certain extent, the ability of displayed CpG danger signals to activate TLR9. Without being bound by theory, this may be because the oligoamines too tightly noncovalently crosslink the danger signals to the walls of the nanocapsule. Further, the oligoamines may change the equilibrium behavior of the i-motif release at pH 5.5. To address this, PNA or other neutral spacers that are uncharged may be used to project the ligands and i-motif domains away from the walls of the nanocapsule, and the sequences of the i-motif may be modified to obtain efficient release in the presence of oligoamines. A neutral spacer can apply enough force to distance the cargo from the walls of the nanocapsule and therefore facilitates efficient release at low pH. In other instances, affinity agents displayed on the outside of the nanocapsule may have reduced activity due to interactions with the nanocapsule wall, for example, after coating with oligoamines. To address this, neutral spacers elements may be included to project out and break unwanted interactions with the nanocapsule wall.

### Example 17

The purpose of this Example is to investigate the ability of engineered DNA nanocapsules to instruct dendritic cells (DCs) to activate T cells. DCs are the most potent professional antigen presenting cells (APCs) that initiate and control Ag-specific T cell responses. DCs ingest, process and present antigens to naïve T cells and can stimulate their proliferation and differentiation into effector T cells. Here, the effects of engineered nanocapsules on DC activation, maturation and antigen presentation, as well as the capacity of these DCs to promote the activation and differentiation of CD4⁺ and CD8⁺ T cells are investigated. The well-established model antigen ovalbumin (OVA) is used because there are defined immunogenic epitopes and transgenic OT1 and OT2 mice that have T cell receptors (TCR) specific for peptides that are CD8⁺ and CD4⁺ T cell epitopes corresponding to OVA257-264 and OVA323-339, respectively (Barnden, M. J., et al. Immunology and Cell Biology 76, 34-40 (1998); and Hogquist, K. A., et al. Immunity 3, 79-86 (1995). Thus, presentation of OT1 peptides by DC to OT1 T cells results in expansion and differentiation of OVA-specific CD8⁺ T cells, whereas presentation of OT2 peptides to OT2 cells leads to OVA-specific CD4⁺ T cell expansion and differentiation. One goal of this Example is to provide engineered nanoparticles that enhance the generation and function of CTL and Th1 and T follicular helper (Tfh) cells, as these cell types promote anti-tumor cytolytic and antibody responses. In vitro assays are used to test the ability of the nanocapsules described in Example 16 to activate BMDCs in culture and then to test the ability of these BMDCs to activate T cells. Next, either activated BMDCs or activated T cells are transferred into mice to confirm their functionality in vivo. The most effective nanoparticles are then delivered directly in vivo and tested for antigen-specific activation of T cells.

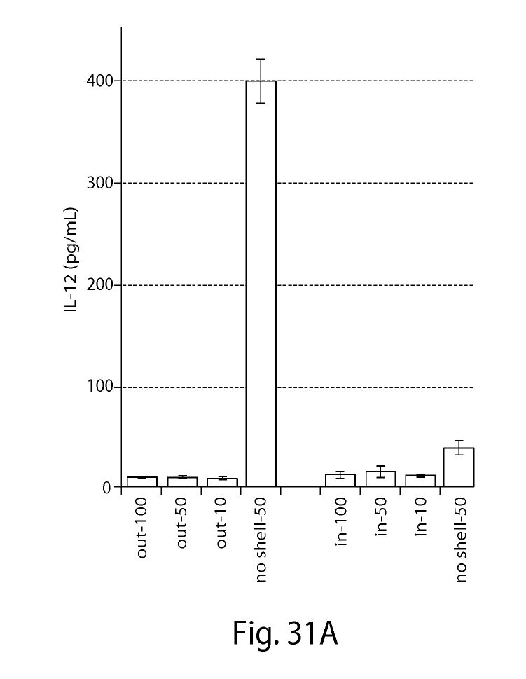

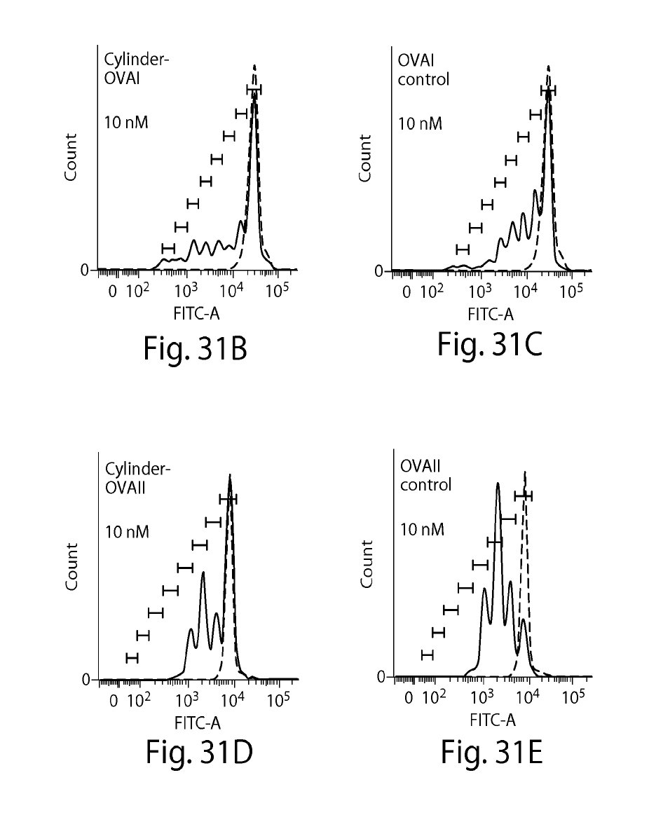

To induce Ag-specific T cell responses, protein antigens need to be phagocytosed, processed and presented on the DC surface as an MHC/peptide complex. The uptake of engineered nanocapsules is compared using Cy5 fluorophores. Nanocapsules are labeled with Cy5 and incubated with DCs for several hours at 4° or 37° C. DCs are washed and fluorescence intensity measured by flow cytometry. The level of fluorescence intensity indicates the efficiency of uptake. BMDC maturation and activation is assessed by determining if the nanocapsules induce cell surface expression of CD80, CD86, MHC I, MHC II and CD40. Another hallmark of DC activation is enhanced secretion of proinflammatory cytokines. The method of DC activation determines the types of cytokines produced (proinflammatory and anti-inflammatory). CD40 stimulation induces IL-6, TNF, IL-15, IL-12 p40, whereas TLR stimulation with LPS induces IL-12p35, IL-1α/β and poly I:C and CpG induce IFN-γ. In particular, IL-12 and IFN-γ play an important role in generating anti-tumor responses to DC vaccines. The secretion of these cytokines as well as anti-inflammatory cytokines (IL-10) is assessed in culture supernatants by cytokine bead arrays (CBA). In addition, proinflammatory signaling pathways are evaluated in these DC by evaluating cell lysates for phosphorylation Ikk α/β, p38, and JNK (associated with IL-12 secretion) and pro-survival molecule Akt. As shown in FIG. 31 (left panel), DNA nanoparticles can induce IL-12 secretion by BMDCs.

To assess BMDC capacity to process and present OT1 or OT2 peptides to T cells and stimulate T cell responses, OT1 or OT2 cells are co-cultured with DCs that have been pre-incubated with engineered nanocapsules for several hours. CD8⁺ OT1 cell activation and cell division is analyzed at 24, 48, 72, and 96 hours after DC priming. As shown in FIG. 31 (right panel) DNA nanoparticles bearing OVA1 or OVA2 can stimulate T cell expansion. Cell division is examined by CFSE dye dilution versus expression of activation and differentiation markers (e.g., CD44, CD25, CD69, KLRG1, CD127), initiation of effector function by evaluating cytokines (IFN-α, TNF, IL-2, Mip1α) by intracellular cytokine staining and cytotoxicity (Granzyme B, perforin) by intracellular staining, as well as transcription factors important for CD8⁺ T cell differentiation (T-bet and Eomes). T cell survival (e.g., bcl-2 positive) is compared with percentages/numbers of T cells at each time point. To test the cytotoxic functionality of these T cells, B16-Ova cells and B16 cells are labeled with different cell surface dyes, and these two cell lines are co-cultured with activated CD8⁺ T cells. To quantitatively assess cytotoxic capacity and specificity, live cells are counted after 48 hours or 72 hours and compared to control wells without CD8⁺ T cells. Likewise, the capacity of the engineered nanocapsules to stimulate activation and differentiation of naive OT2 CD4+ T cells into effector cell subsets is compared. OT2 cell activation and division is assessed using the approaches detailed for OT1 cells. CD4⁺ cells can differentiate into different subclasses of effector cells defined by distinct expression of cell surface molecules, transcription factors and cytokines. Cytokines and transcription factors that characterize Th1 (IL-2, IFN-α, t-bet, stat1,4), Th2 (IL-4, 5, 10, 13, gata-3, stat6), Th17 (IL-17, IL-22, stat3, rorc), and Tfh (IL-21, bcl-6) cells are measured. In addition, the ability of these BMDC to promote induction of anti-inflammatory regulatory T cells from naïve T cells is assessed. Engineered particles loaded with OT1 or OT2 peptide, OVA protein or control protein (hen egg lysozyme) are compared.

A subset of engineered nanocapsules, which stimulate OT1 and OT2 cell proliferation and Th1 responses in vitro, are selected. CFSE-labeled OT1 or OT2 cells are transferred intravenously into naïve mice and the mice are immunize subcutaneously with engineered nanocapsules (containing OVA peptide antigens or control). Ex vivo versus in vivo activation of DCs is compared, and T cell proliferation by CFSE dye dilution is evaluated. An in vivo CTL assay is used to investigate whether the activated OT1 cells can differentiate into effective CTL and kill Ag-specific target cells (OT1 peptide-pulsed splenocytes). Mice are immunized with nanocapsules containing OVA peptide or control peptide, or OVA protein in adjuvant (as a positive control), and killing of labeled syngeneic splenocytes pulsed with (CFSE) or without (Cell-Trace Violet) OT1 peptide is compared.

In instances where binding strength of the affinity agents displayed on the outer surface of the nanocapsules may be reduced by tethering to the nanocapsule, the affinity agents can be projected further from the surface by including stiff (e.g., dsDNA, PNA) or flexible (e.g., PEG) linkers. Protein ligands can be expressed as covalent conjugates to SNAP tag domains to achieve a degree of clearance from the nanocapsule surface.

In instances wherein a nanocapsule may not open up and release its cargo within endosomal cell compartments as efficiently as observed in acellular experiments due, for example, to differences in the respective environments, intracellular opening of the nanocapsules between the top and bottom halves can be assayed by FRET. Alternatively, pH 5.5 triggered opening of the holes can be programmed in the top of the domes, through which released cargo can diffuse out. This can be achieved by introducing i-motif sequences tethering the plug to the inner walls of the nanocapsule. Upon exposure to low pH, the i-motif tethers will coil up, generating force to retract the plug from its hole.

### Example 18

The purpose of this Example is to test the efficacy of a selected subset of engineered nanocapsules as tumor vaccines. Nanocapsules are tested as prophylactic and therapeutic vaccines by vaccinating before or after tumor implantation. The therapeutic efficacy is assessed initially in the B16 OVA melanoma, and then extended to the Her2 neu and 4T1 breast cancer models. Results are compared to a traditional vaccine (e.g., target peptide in CFA). To assay for in vivo effectiveness, tumor growth and composition of the immune infiltrate is monitored, and antigen-specific T-cell activation, proliferation, and effector functions in the tumor microenvironment, as well as draining lymph nodes and spleen are analyzed.

Vaccination.

For prophylactic vaccinations, engineered nanocapsules are implanted subcutaneously in one flank and challenged 2 weeks later with subcutaneous injection of 10⁵-10⁶ tumor cells on the other flank. To assess the efficacy of therapeutic vaccination, mice are implanted with 10⁵-10⁶ tumor cells subcutaneously in one flank. When tumors reach ˜100 mm³ in volume, engineered nanocapsules are given subcutaneously in the other flank or intratumorally. A subset of mice receive weekly vaccinations for up to three weeks. As a positive control in both cases, tumor peptides (e.g., the OTI peptide) are injected in CFA instead of nanoparticles in control tumor-bearing mice at the same time as the initial nanoparticle vaccination.

Analyses of Tumor Growth.

B16 OVA cells are implanted subcutaneously into C57BL/6 mice, and Her2/neu or 4T1 cells subcutaneously into BALB/c mice. Tumor growth and survival are compared. Tumor volumes are measured three times weekly. The mice are monitored for tumor ulceration and signs of toxicity, particularly >20% weight loss and other signs of treatment-induced stress. The endpoint for the survival study is tumor size (limit of 1 cm³) or ulcerated/necrotic tumors, in compliance with our IACUC regulations. The following cohorts of mice (n=8-10 mice per group) are compared: (1) mock treatment, (2) nanocapsular vaccine with relevant Ag, (3) nanocapsular vaccine with no Ag; (4) positive control vaccine.

Cellular Immunologic Analyses.

The effects of the nanocapsules on immune responses are examined, locally at the vaccine site, in the draining lymph node and within the tumor over time. Cellular immunologic, molecular and immunohistologic approaches are used to analyze both DCs and T cells.

Dendritic cell subsets are first compared by flow cytometry: CD11c⁺ MHC class II^{hi} cells expressing CD103, CD8a (e.g., cross-presenting), PDCA (e.g., plasmacytoid) or CD11b (e.g., monocyte-derived). DC maturation status (e.g., CD80, CD86, CD40, CD137L) and expression of inhibitory ligands (e.g., PD-L1 and PD-L2) are examined, other myeloid cells including monocyte/macrophages, neutrophils and myeloid derived suppressor cells are characterized using appropriate markers (e.g., Mer, CD32, F4/80, Ly6C, Ly6G), and expression of immunoregulatory markers (e.g., arginase, indoleamine 2,3 digoxygenase, CD39/CD73) is assessed. Cytokines expressed in tissue extracts are examined, and tumor protective (e.g., IFNy, TNF, IL-12, IL-18), tumor promoting (e.g., IL-6, IL-17, IL-23), and immunosuppressive (e.g., IL-10 and TGFβ) cytokines are examined using luminex based assays and qRTPCR.

To determine the effects of nanocapsule vaccination on CD8⁺ T cell responses, methods described in Example 17 are used. In addition, adoptive-transfer of antigen-specific T cells (e.g., OT1 T cells with B16 Ova tumors) as well as tetramers are used to probe endogenously generated antigen-specific T cells. In addition to the activation markers and cytokines discussed in Example 17, percentages/numbers of CD8⁺ T cells, expression of inhibitory receptors (e.g., PD-1, LAG-3, TIM-3, CD160), and proliferation (e.g., Ki-67 expression or BrDu incorporation) are assessed. Ex vivo assays (cytotoxicity assays, ELISPOT) are used to evaluate functionality.

FoxP3⁺ Treg, which inhibit anti-tumor immunity is also assessed. The following is also assessed: 1) percentages/numbers, 2) proliferation (Ki67, BrDU) and survival (Bcl2), 3) markers of function (e.g., IL-10, IRF4) and/or differentiation to a more effector phenotype (e.g., Tbet, IFNα), and 4) markers of stability (e.g., repressed pAkt or increased neuropilin, Helios and Bcl2) by flow cytometry. The CD8+/Treg ratio, as an increase correlates with enhanced anti-tumor immunity, is determined.

Analyses of Humoral Immunity.

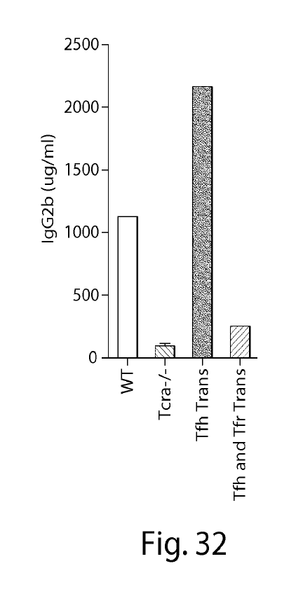

Increasing evidence suggests that successful anti-tumor therapies promote both CTLs and humoral immunity, and work is needed to understand why tumor vaccines generally do not elicit potent long-lived antibody responses. B cells are stimulated to produce protective antibodies by T follicular helper cells (Tfh) and inhibited by T follicular regulatory cells (Tfr), though it is not yet clear whether Tfr cells suppress B cell responses to tumors. To test if eliminating Tfr cells leads to heightened B cell responses to melanoma antigens, mice were immunized with a BRAF/PTEN (a melanoma cell line (Cooper, Z. A. et al. Cancer Immunol Res 2, 643-654 (2014))) tumor lysate emulsified in CFA, and 7 days later, Tfh and Tfr cells from these mice were sorted, and Tfh and/or Tfr cells transferred to Tcrα^{-/-} recipients (that lack Tfh and Tfr cells). These recipients were given BRAF/PTEN tumors and 8 days later antibody levels were measured. As shown in FIG. 32, Tfh cells stimulated antibody production, but addition of Tfr cells potently suppressed antibody production. These studies indicate that Tfr cells can inhibit tumor immunity in vivo.

To investigate how nanocapsular vaccines modulate humoral immune responses and Tfh and Tfr cells, Tfh, Tfr and B cell responses are compared to a model antigen (OVA), tumor cells expressing this antigen (B16 OVA) alone or B16 OVA plus nanocapsule vaccine. B16 OVA cells are implanted subcutaneously, and vaccinated with nanocapsule vaccine as above. Seven or 14 days later the following are assessed: (1) Tfh, Tfr and B cells numbers as well as the Tfh/Tfr ratio, as this ratio rather than absolute cell numbers affects antibody production as well as Tfh, Tfr and B cells proliferation (by Ki67 expression or BrdU uptake); (2) expression of CXCR5 (the chemokine that directs these cells to B cell zones), Bcl6 (master transcription factor for Tfh cells), Blimp-1 (transcriptional repressor of Tfh cells) on Tfh and Tfr cells and cytokines (IL-21 and IL-4, which stimulate B cell function) in Tfh cells; (3) germinal center B cells, plasma cells, memory B cells and serum antibody levels (total and tumor-specific using ELISA and tumor lysates) to determine B cell responses; and (4) whether autoantibodies are elicited by the tumors.

Similar studies are conducted using the Her2/neu cell line, which elicits both T and B cell responses, providing a valuable tool for studying the effect of therapeutic strategies on antitumor humoral as well as CD8⁺ T cell responses. Tfh, Tfr and B cell responses are evaluated, as described for the B16 OVA studies, and serum antibodies (IgM, IgG total and subtypes, IgA) to Her2/neu are analyzed using Her2/neu expressing cell lines. Similar studies using the 4T1 cell line are also conducted.

Combination with Checkpoint Blockade.

The fundamental concept underlying tumor vaccines is that endogenous DCs have failed to stimulate a sufficient anti-tumor T cell response when a tumor is present. However, even with sufficient T cell priming by DCs, T cells can be suppressed both in and out of the tumor microenvironment. Checkpoint blockade aims to remove one of the nodes of this suppression, and the current paradigm is that it fails if the anti-tumor immune response is insufficient at the initiation of treatment. Thus, without being bound by theory, it is thought that a combination of the nanocapsular vaccination approach with checkpoint blockade will lead to a synergy, first by inducing a strong anti-tumor immune response (nanocapsular vaccination) and then by preventing suppression of T cells (checkpoint blockade). A nanocapsular vaccination approach is combine with anti-PD-1 or anti-CTLA-4 blocking antibodies. The relative timing of the vaccination and checkpoint blockade (simultaneous or sequential vaccination followed by checkpoint blockade) are tested to investigate if this impacts the therapeutic response. An analysis of how combination therapy affects tumor growth and mouse survival, as well as T cell responses (focusing on CD8⁺ T cells and Treg), is conducted using the approaches described above.

Analyses of Adverse Events.

Many of the pathways that inhibit anti-tumor immunity also are critical for T cell tolerance and preventing autoimmunity. Blockade of some pathways, such as CTLA-4 (ipilumimab), leads to serious autoimmune reactions. Thus, it is important to assess the safety of each vaccine approach. For these reasons, treated mice are evaluated for evidence of autoimmunity and immunopathology. Non-tumor tissues such as gut, liver and lung are also analyzed for evidence of inflammatory infiltrates (e.g., colitis). Systemic application of high dose TLR ligands not only can induce DC maturation, but also high levels of serum cytokines (IL-6, IL-8, type 1 interferons), which can lead to toxic shock like effects. Therefore, serum levels of these cytokines are also evaluated.

Nanocapsular vaccines alone, in some instances, may not elicit a strong anti-tumor response because some tumors, such as the B16 melanoma and Her2/neu breast carcinoma, are weakly immunogenic tumors. However, antigen-loaded matrices with GM-CSF in combination with TLR agonists (CpG-ON, MPLA and poly I:C) have led to significant protection in the B16 melanoma model. In some embodiments, nanocapsules that promote the most potent anti-tumor immunity (slowing of tumor growth and/or enhanced intratumoral T cell responses) are combine with checkpoint blockade for synergy studies.

### Materials and Methods

Folding of DNA Nanostructures

Single stranded scaffold DNA was prepared in-house using standard plasmid expression protocol. Staple DNA strands were from Integrated DNA Technology. To assemble the structures, unpurified DNA staple strands were mixed in 10 fold excess with the scaffold at a concentration of 10 nM in 0.5×TE buffer (5 mM Tris, pH 7.9, 1 mM EDTA) supplemented with 6 to 18 mM MgCl₂.

Annealing Ramps

The strand mixture was then annealed in a polymerase chain reaction (PCR) thermo cycler by a denaturation step at 65° C. over 15 minutes, then a 24-hour or 72-hour linear cooling ramp from 50° C. to 24° C. The annealing can also be done isothermally at a given temperature, specific to the structure being folded.

Agarose gel analysis: Annealed samples were then subjected to 2% native agarose gel electrophoresis at 70 volts for 2 hours (gel prepared in 0.5×TBE buffer supplemented with 11 mM MgCl₂ and 0.005% (v/v) ethidium bromide (EtBr)) in an ice water bath. Then, the target gel bands were excised and placed into a Freeze 'N Squeeze column (Bio-Rad Laboratories, Inc.). The gel pieces were crushed into small pieces by a microtube pestle in the column, and the column was then centrifuged at 7000 g for 5 minutes. Samples that were extracted through the column were collected for TEM or atomic force microscopy (AFM) imaging.

Imaging

For imaging, 3.5 μL of agarose-gel-purified or unpurified sample was adsorbed for 1 minute onto glow-discharged, carbon-coated TEM grids. The grids were then stained for 20 seconds using a 2% aqueous uranyl formate solution containing 25 mM NaOH. Imaging was performed using a JEOL JEM-1400 microscope operated at 80 kV.

Incubation with Polylysine Polymers

Polylysine peptides were from Peptide 2.0. They were solubilized in 0.5×TE buffer (5 mM Tris, pH 7.9, 1 mM EDTA containing 10 mM Mg) and aliquoted at various concentrations and stored at −20° C. 100 μL of purified DNA nanostructures at 2 nM concentration in 0.5×TE buffer (5 mM Tris, pH 7.9, 1 mM EDTA, 10 mM Mg) was incubated with x nM amount of polylysine (x depends on the length of the polylysine used) for 1 hour. The sample was then transferred into a micro dialysis unit, purchased from Thermo Scientific (Slide-A-Lyzer). The dialysis units were placed in 1 L of 0.5×TE buffer (5 mM Tris, pH 7.9, 1 mM EDTA) and dialyzed for 24 hours. There was no magnesium in the dialysis buffer.

Dialyzed samples were then subjected to 2% native agarose gel electrophoresis at 70 volts for 2 hours (gel prepared in 0.5×TBE buffer and 0.005% (v/v) ethidium bromide (EtBr)) in an ice water bath. Only the DNA nanostructures that survive dialysis migrate on a no-magnesium gel.

Dialyzed samples were also characterized by imaging. For imaging, 3.5 L of dialyzed sample was adsorbed for 1 minute onto glow-discharged, carbon-coated TEM grids. The grids were then stained for 20 seconds using a 2% aqueous uranyl formate solution containing 25 mM NaOH. Imaging was performed using a JEOL JEM-1400 operated at 80 kV.

Selection of Animal Models.

The experiments in Example 18 employ three transplantable melanoma and breast carcinoma tumor models in which tumor cells are implanted subcutaneously into syngeneic immunocompetent mice. Transplantable tumor models have provided instructive insights for cancer immunotherapy and are easier to use compared to genetically engineered transgenic tumor models, whose complexity limits the number of experimental parameters that can be examined. The B16 melanoma cell line is used, which has been extensively used for clinical development of tumor cell vaccines (Dranoff, G. Nat Rev Immunol 12, 61-66 (2012); Quezada, S. A., et al. J Clin Invest 116, 1935-1945 (2006)), as well as for evaluating the consequences of immunoinhibitory pathway blockade (e.g., antibodies to CTLA-4 and PD-1) (Dranoff, G. Nat Rev Immunol 12, 61-66 (2012)). The translational utility of the B16 melanoma model is illustrated by the recent FDA approvals of anti-CTLA-4 and anti-PD-1 antibodies for treating advanced melanoma. In addition, a novel Her2/neu breast cancer cell line from a transgenic model of Her2/neu breast cancer (Black, C. M., et al. Cancer Immunol Res 2, 307-319 (2014)) is used. This Her2/neu cell line forms tumors upon implantation into syngeneic Balb/c mice, elicits both T and B cell responses, and responds to immunotherapy. Thus, the Her2/neu cell line provides a valuable tool for studying the effects of therapeutic strategies on anti-tumor humoral as well as CD8⁺ T cell responses. The 4T-1 breast cancer cell line is also used to test the most potent vaccine strategies, as this line is at least partially sensitive to checkpoint blockade and cancer vaccination (Kim, K. et al. Proc Natl Acad Sci USA 111, 11774-11779 (2014)).

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃alkyl”). In some embodiments, an alkyl group has 1 to 2 carbon atoms (“C₁₋₂alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetra-hydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic, tricyclic) 4n+2 aromatic ring system (e.g., having 6, 10, or 14 it electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀alkynyl, C₃₋₁₀carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆alkyl, —P(═O)₂(C₁₋₆ alkyl), —P(═O)(C₁₋₆alkyl)₂, —OP(═O)(C₁₋₆alkyl)₂, —OP(═O)(OC₁₋₆alkyl)₂, C₁₋₆ alkyl, C₁₋₆ perhaloalkyl, C₂₋₆alkenyl, C₂₋₆alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-I-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), J3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The invention is not intended to be limited in any manner by the above exemplary listing of substituents.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “quaternary salt” refers to a nitrogen atom directly attached to or part of the parent compound or parent chain which comprises two to four substituents or groups attached thereto such that the nitrogen has a valency of four, wherein the nitrogen atom is positively charged, and the charge is balanced with a counteranion. Exemplary quaternary salts include but are not limited to a substituent amine attached to the parent compound or chain —N(R^{bb})₃⁺X⁻ or an amine part of the parent chain —N(R^{bb})₂—⁺X⁻, wherein R^{bb} and X⁻ are as defined herein.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits and/or methods, if such features, systems, articles, materials, kits and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A nucleic acid nanostructure subsaturated with polyamine polymers.

2. The nucleic acid nanostructure of claim 1, wherein the nucleic acid nanostructure comprises deoxyribonucleic acid (DNA).

3. The nucleic acid nanostructure of claim 2, wherein the DNA is linear, single-stranded plasmid DNA.

4. The nucleic acid nanostructure of claim 3, wherein the linear, single-stranded plasmid DNA is linear, single-stranded M13 plasmid DNA.

5. The nucleic acid nanostructure of any one of claims 1-4, wherein the nucleic acid nanostructure is a three-dimensional nucleic acid nanostructure.

6. The nucleic acid nanostructure of any one of claims 1-5, wherein the nucleic acid nanostructure is rationally designed.

7. The nucleic acid nanostructure of any one of claims 1-6, wherein the nucleic acid nanostructure does not include condensed nucleic acid.

8. The nucleic acid nanostructure of any one of claims 1-7, wherein the nucleic acid nanostructure does not include circular plasmid DNA.

9. The nucleic acid nanostructure of any one of claims 1-8, wherein the nucleic acid nanostructure is a non-coding nucleic acid nanostructure.

10. The nucleic acid nanostructure of any one of claims 1-9, wherein the nucleic acid nanostructure is not encapsulated or coated with lipids.

11. The nucleic acid nanostructure of any one of claims 1-10, wherein less than 95% of phosphates of the nucleic acid nanostructure are linked to amines of the polyamine polymers.

12. The nucleic acid nanostructure of claim 11, wherein less than 90% of phosphates of the nucleic acid nanostructure are linked to amines of the polyamine polymers.

13. The nucleic acid nanostructure of claim 12, wherein less than 80% of phosphates of the nucleic acid nanostructure are linked to amines of the polyamine polymers.

14. The nucleic acid nanostructure of claim 13, wherein less than 70% of phosphates of the nucleic acid nanostructure are linked to amines of the polyamine polymers.

15. The nucleic acid nanostructure of claim 14, wherein less than 60% of phosphates of the nucleic acid nanostructure are linked to amines of the polyamine polymers.

16. The nucleic acid nanostructure of any one of claims 1-15, wherein the polyamine polymers comprise amino acids.

17. The nucleic acid nanostructure of claim 16, wherein the amino acids contain amine-containing side chains.

18. The nucleic acid nanostructure of claim 17, wherein the amino acids comprise lysine.

19. The nucleic acid nanostructure of any one of claims 16-18, wherein the polyamine polymers comprise peptides.

20. The nucleic acid nanostructure of claim 19, wherein the peptides comprise at least 10% lysine.

21. The nucleic acid nanostructure of claim 20, wherein the peptides comprise at least 25% lysine.

22. The nucleic acid nanostructure of claim 21, wherein the peptides comprise at least 50% lysine.

23. The nucleic acid nanostructure of claim 22, wherein the peptides comprise at least 75% lysine.

24. The nucleic acid nanostructure of claim 23, wherein the peptides comprise at least 90% lysine.

25. The nucleic acid nanostructure of any one of claims 18-24, wherein lysines of the polyamine polymers are separated from each other by at least one non-lysine amino acid.

26. The nucleic acid nanostructure of claim 25, wherein lysines of the polyamine polymers are separated from each other by at least two non-lysine amino acids.

27. The nucleic acid nanostructure of claim 26, wherein lysines of the polyamine polymers are separated from each other by at least three non-lysine amino acids.

28. The nucleic acid nanostructure of any one of claims 1-24, wherein the polyamine polymers are polylysine homopolymers.

29. The nucleic acid nanostructure of any one of claims 1-28, wherein the polyamine polymers are branched.

30. The nucleic acid nanostructure of any one of claims 16-29, wherein the polyamine polymers comprise 4 to 100 amino acids.

31. The nucleic acid nanostructure of claim 30, wherein the polyamine polymers comprise 4 to 75 amino acids.

32. The nucleic acid nanostructure of claim 31, wherein the polyamine polymers comprise 4 to 50 amino acids.

33. The nucleic acid nanostructure of claim 32, wherein the polyamine polymers comprise 4 to 25 amino acids.

34. The nucleic acid nanostructure of claim 33, wherein the polyamine polymers comprise 4 to 15 amino acids.

35. The nucleic acid nanostructure of claim 34, wherein the polyamine polymers comprise 6 amino acids.

36. The nucleic acid nanostructure of claim 34, wherein the polyamine polymers comprise 8 amino acids.

37. The nucleic acid nanostructure of claim 34, wherein the polyamine polymers comprise 10 amino acids.

38. The nucleic acid nanostructure of claim 34, wherein the polyamine polymers comprise 12 amino acids.

39. The nucleic acid nanostructure of any one claims 1-27 or 29-38, wherein the polyamine polymers comprise:
one or more groups of Formula (I) or a pharmaceutically acceptable and/or quaternary salt thereof covalently attached thereto: 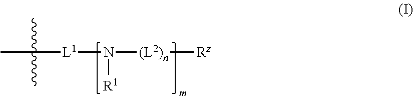
or one or more compounds of Formula (II) or a pharmaceutically acceptable salt and/or quaternary thereof non-covalently associated therewith: 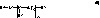
wherein:
L¹ is a direct covalent bond or a linker group comprising any one or combination of optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, and optionally substituted heteroarylene;
each R¹ is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —C(═O)R^{A}, —C(═O)OR^{A}, —C(═O)N(R^{A})₂, or a nitrogen protecting group; or
R¹ is a group of formula: 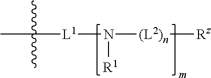
each L² is independently a linker selected from any one or combination of optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, and optionally substituted heteroarylene;
each R^{z} is independently hydrogen, —N(R^{A})₂, —OR^{A}, —SR^{A}, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting if attached to a nitrogen atom, an oxygen protecting group if attached to an oxygen atom, or a sulfur protecting group if attached to a sulfur atom;
each R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting if attached to a nitrogen atom, an oxygen protecting group if attached to an oxygen atom, or a sulfur protecting group if attached to a sulfur atom, or two R^{A} groups attached to a nitrogen atom are joined to form an optionally substituted heterocyclic ring or optionally substituted heteroaryl ring.
n is an integer between 1 and 100,000, inclusive; and
m is an integer between 1 and 100,000, inclusive.

40. The nucleic acid nanostructure of claim 39, wherein L² is an optionally substituted alkylene, optionally substituted alkenylene, or optionally substituted alkynylene.

41. The nucleic acid nanostructure of claim 40, wherein L² is an optionally substituted alkylene of formula —(CH₂)ₚ—, wherein p is an integer between 1 and 10, inclusive.

42. The nucleic acid nanostructure of claim 39, wherein L² is —C(R^{A})—C(═O)—.

43. The nucleic acid nanostructure of claim 42, wherein R^{A} of —C(R^{A})—C(═O)— is an optionally substituted alkyl.

44. A nucleic acid composition, comprising:
the nucleic acid nanostructure of any one of claims 1-43, 49 and 50; and
a solution that comprises less than 10 mM magnesium (Mg²⁺).

45. The nucleic acid composition of claim 44, wherein the solution comprises 0.1 mM to 0.9 mM magnesium (Mg²⁺).

46. The nucleic acid composition of claim 44 or 45, wherein the solution comprises 0.6 mM (Mg²⁺).

47. The nucleic acid composition of any one of claims 44-46, wherein the solution further comprises 0.5 mM to 1.5 mM calcium (Ca²⁺).

48. The nucleic acid composition of claim 47, wherein the solution further comprises 0.9 mM Ca²⁺.

49. The nucleic acid nanostructure of any one of claims 1-43 further comprising poly(ethylene imine)-polyethylene glycol (PEI-PEG) copolymers.

50. The nucleic acid nanostructure of claim 49, wherein the nucleic acid nanostructure is subsaturated with PEI-PEG copolymers.

51. A nucleic acid nanocapsule comprising:
a first nucleic acid nanostructure linked to a second nucleic acid nanostructure through a pH-sensitive interface; and
an interior compartment formed by linkage of the first nucleic acid nanostructure to the second nucleic acid nanostructure.

52. The nucleic acid nanocapsule of claim 51, wherein the first nucleic acid nanostructure comprises pH-sensitive single-stranded nucleic acid handles, and the second nucleic acid nanostructure comprises single-stranded nucleic acid anti-handles that are partially complementary to the pH-sensitive handles, and the first nucleic acid nanostructure is linked to the second nucleic acid nanostructure through hybridization of the pH-sensitive handles to the anti-handles.

53. The nucleic acid nanocapsule of claim 52, wherein the pH-sensitive handles comprise the sequence of SEQ ID NO: 1.

54. The nucleic acid nanocapsule of claim 52 or 53, wherein the anti-handles comprise the sequence of SEQ ID NO: 2 or SEQ ID NO: 3.

55. The nucleic acid nanocapsule of any one of claims 51-54, wherein the nanocapsule has two ends, and each end of the nanocapsule has an opening of less than 10 nm in diameter.

56. The nucleic acid nanocapsule of claim 55, wherein each end of the nanocapsule has an opening of 2 nm to 10 nm in diameter.

57. The nucleic acid nanocapsule of any one of claims 51-56, further comprising an agent linked to an interior surface and/or an exterior surface of the nanocapsule.

58. The nucleic acid nanocapsule of claim 57, further comprising at least two agents linked to an interior surface and/or an exterior surface of the nanocapsule.

59. The nucleic acid nanocapsule claim 56 or 57, wherein the agent is linked to an interior surface and/or an exterior surface of the nanocapsule through hybridization of complementary single-stranded nucleic acids.

60. The nucleic acid nanocapsule claim 59, wherein the agent is bound to an interior surface and/or an exterior surface of the nanocapsule through hybridization of partially complementary pH-sensitive handles and anti-handles.

61. The nucleic acid nanocapsule of any one of claims 57-60, wherein the agent is a targeting molecule linked to the exterior surface.

62. The nucleic acid nanocapsule of claim 61, wherein the targeting molecule is an antibody, an antibody fragment or a ligand.

63. The nucleic acid nanocapsule of any one of claims 57-60, wherein the agent is a therapeutic agent, a prophylactic agent, a diagnostic agent and/or an adjuvant.

64. The nucleic acid nanocapsule of any one of claims 57-60, wherein the agent is an antigen.

65. The nucleic acid nanocapsule of claim 64, wherein the antigen is a peptide antigen.

66. The nucleic acid nanocapsule of any one of claims 57-60, wherein the agent is an adjuvant.

67. The nucleic acid nanocapsule of claim 66, wherein the adjuvant is CpG oligonucleotides.

68. The nucleic acid nanocapsule of any one of claims 51-67, further comprising polyamine polymers and/or copolymers of cationic poly(ethylene imine) and polyethylene glycol.

69. The nucleic acid nanocapsule of claim 68, wherein the nanocapsule is subsaturated with the polyamine polymers and/or copolymers of cationic poly(ethylene imine) and polyethylene glycol.

70. The nucleic acid nanocapsule of any one of claims 51-69, wherein the first nucleic acid nanostructure and/or the second nucleic acid nanostructure is in the form of a cylinder.

71. A composition comprising the nucleic acid nanocapsule of any one of claims 51-70.

72. The composition of claim 71, further comprising a delivery vehicle.

73. The composition of claim 72, wherein the delivery vehicle is a polymeric gel.

74. A method comprising administering to a subject the nucleic acid nanocapsule of any one of claims 51-70 or the composition of any one of claims 71-73.

75. A method comprising delivering to cells the nucleic acid nanocapsule of any one of claims 51-70.

76. A kit comprising
a first nucleic acid nanostructure comprising pH-sensitive single-stranded nucleic acid handles; and
a second nucleic acid nanostructure comprising single-stranded nucleic acid anti-handles that are partially complementary to the pH-sensitive handles,
wherein in an aqueous solution having a pH of greater than 6, the first nucleic acid nanostructure attaches to the second nucleic acid nanostructure through hybridization of the pH-sensitive handles to the anti-handles, thereby forming a nucleic acid nanocapsule having an internal compartment.

77. The kit of claim 76, further comprising an aqueous solution having a pH of greater than 6.

78. The kit of claim 76 or 77, wherein the first and second nucleic acid nanostructures comprise pH-sensitive handles.

79. The kit of any one of claims 76-78, wherein the pH-sensitive handles comprise the sequence of SEQ ID NO: 1.

80. The kit of claim 78 or 79, further comprising an agent linked to anti-handles that are partially complementary to the pH-sensitive handles.

81. The kit of claim 76 or 77, further comprising pH-sensitive handles and agents linked to anti-handles that are partially complementary to the pH-sensitive handles.


SEQUENCE LISTING (8 sequences)

SEQ ID NO: 1 - DNA, 21 bp, Artificial Sequence
  Synthetic Polynucleotide
ccctaaccctaaccctaaccc

SEQ ID NO: 2 - DNA, 21 bp, Artificial Sequence
  Synthetic Polynucleotide
ttgttagtgttagtgttaggg

SEQ ID NO: 3 - DNA, 21 bp, Artificial Sequence
  Synthetic Polynucleotide
ggtttattgttaggtttagtt

SEQ ID NO: 4 - protein, 8 aa, Artificial Sequence
  Synthetic Polypeptide
WSRISLSR

SEQ ID NO: 5 - protein, 12 aa, Artificial Sequence
  Synthetic Polypeptide
YFKWSRISLSRY

SEQ ID NO: 6 - protein, 37 aa, Artificial Sequence
  Synthetic Polypeptide
LLGDFFRKSKEKIGKEFKRIVQRIKDFLRNLVPRTES

SEQ ID NO: 7 - protein, 6 aa, Artificial Sequence
  Synthetic Polypeptide
KKKKKK

SEQ ID NO: 8 - protein, 6 aa, Artificial Sequence
  Synthetic Polypeptide
RRRRRR